(19) United States
(12) Patent Application Publication
(10) Pub. No.: US 2026/0258394 A1
(43) Pub. Date: Sep. 3, 2026

(54) PLANT ENZYMES WITH IMPROVED ACTIVITY

(71) Applicant: MAX-PLANCK-GESELLSCHAFT ZUR FÖRDERUNG DER WISSENSCHAFTEN E.V., München (DE)

(72) Inventors: Tobias ERB, Marburg (DE); Daniel MARCHAL, Marburg (DE)

(73) Assignee: MAX-PLANCK-GESELLSCHAFT ZUR FÖRDERUNG DER WISSENSCHAFTEN E.V., München (DE)

(21) Appl. No.: 19/489,263

(22) PCT Filed: May 31, 2024
(86) PCT No.: PCT/EP2024/064999

§ 371 Date: Dec. 1, 2025

(30) Foreign Application Priority Data
Jun. 2, 2023 (EP) 23176985.2

(51) Int. Cl. C12N 9/00; C12N 1/20; C12N 15/70; C12R 1/19

(52) U.S. Cl. CPC C12N 9/93; C12N 1/20; C12N 15/70; C12R 2001/19

(57) ABSTRACT

The present invention provides a Glycolyl-CoA Carboxylase (GCC), wherein said GCC is characterized in that: it comprises an amino acid sequence having at least 60% sequence identity to SEQ ID NO: 1, and it has one or more amino acid substitutions, deletions or insertions at a position selected from the group consisting of positions 20 and 100 in the amino acid sequence shown in SEQ ID NO: 1 or at a position corresponding to any of these positions, preferably wherein the GCC has an improved activity over a reference GCC, preferably wherein the reference GCC comprises the amino acid sequence of SEQ ID NO: 1.

The present invention provides a Glycolyl-CoA Carboxylase (GCC), wherein said GCC is characterized in that: it comprises an amino acid sequence having at least 60% sequence identity to SEQ ID NO: 1, and it has one or more amino acid substitutions, deletions or insertions at a position selected from the group consisting of positions 20 and 100 in the amino acid sequence shown in SEQ ID NO: 1 or at a position corresponding to any of these positions, preferably wherein the GCC has an improved activity over a reference GCC, preferably wherein the reference GCC comprises the amino acid sequence of SEQ ID NO: 1.

Photosynthesis plays a crucial role in the global carbon cycle. It converts CO₂ into organic compounds that are used as feedstock by heterotrophic organisms. However, the efficiency of photosynthesis via the Calvin-Benson cycle is diminished by RuBisCO's inability to reliably distinguish between O₂ and CO₂. Photorespiration that is required to recycle 2-phosphoglycolate, the product of RuBisCO's oxygenation side reaction, involves the undesired loss of carbon. The inventors recently developed the Tartronyl-CoA (TaCo) pathway, a synthetic carboxylation module circumventing this loss of carbon and instead fixing additional CO₂ (see inter alia WO 2016/207219). It requires less adenosine triphosphate (ATP) and less reducing equivalents to produce biomass and is thereby assumed to improve growth rates and carbon yield of photosynthetic organisms. Structure-guided enzyme engineering coupled with large-scale screening of mutagenesis libraries was applied to evolve Glycolyl-CoA Carboxylase (GCC M5), the key enzyme of the pathway. In an iterative engineering workflow five mutations were introduced to convert a former propionyl-CoA carboxylase (PCC) into GCC M5 that allows the carboxylation of glycolyl-CoA at a catalytic rate of 5.6±0.3 s⁻¹ which is comparable to natural carboxylases (Scheffen, Nat. Catal. 4, 105-115, 2021). However, GCC M5 has an ATP per carboxylation ratio of about 4 although the theoretical ratio is 1.

Thus, there is a need to provide means and methods to improve the activity of CO₂ fixing enzymes, in particular Glycolyl-CoA Carboxylase.

The technical problem underlying the present invention is the provision of a Glycolyl-CoA Carboxylase with improved activity.

The technical problem is solved by provision of the embodiments characterized in the claims and as provided herein below. Specifically, the technical problem is solved, and the above-mentioned difficulties are overcome by the provision of a Glycolyl-CoA Carboxylase (GCC) with certain amino acid substitutions. The inventors determined that amino acid substitutions at positions 20 and 100 in the GCC of SEQ ID NO: 1 result in improved activity.

Accordingly, the present invention provides a Glycolyl-CoA Carboxylase (GCC), wherein said GCC is characterized in that: it comprises an amino acid sequence having at least 60% sequence identity to SEQ ID NO: 1, and it has one or more amino acid substitutions, deletions, or insertions at a position selected from the group consisting of positions 20 and 100 in the amino acid sequence shown in SEQ ID NO: 1 or at a position corresponding to any of these positions, preferably wherein the GCC has an improved activity over a reference GCC, preferably wherein the reference GCC has the amino acid sequence of SEQ ID NO: 1.

The above is illustrated in the appended examples.

In the following the invention is described in more detail.

The present inventors found positions in GCC where amino acid substitutions improve specific activities for glycolyl-CoA carboxylation as well as ATP/CO₂-ratios of GCC. The present inventors could demonstrate that certain amino acid substitutions improve the specific activity for glycolyl-CoA carboxylation as well as ATP/CO₂-ratios of GCC (Table 6). In particular the inventors showed that the amino acid substitution G20R has a 2-3 fold increase in the carboxylation activity (i.e. conversion of glycolyl-CoA into tartronyl-CoA) (FIG. 1). Furthermore, the inventors showed that the amino acid substitution S100N (relative to GCC M5; SEQ ID NO: 1) has a reduced ATP consumption of more than 50% (FIG. 2). In other words, the substitution resulted in improved energetic efficiency.

The invention is characterized by the following items:
1. A Glycolyl-CoA Carboxylase (GCC), wherein said GCC is characterized in that: it comprises an amino acid sequence having at least 60% sequence identity to SEQ ID NO:1, and
   it has one or more amino acid substitutions, deletions or insertions at a position selected from the group consisting of positions 20 and 100 in the amino acid sequence shown in SEQ ID NO: 1 or at a position corresponding to any of these positions,
   preferably wherein the GCC has an improved activity over a reference GCC, preferably wherein the reference GCC comprises the amino acid sequence of SEQ ID NO:1.
2. The GCC of item 1, wherein
   (1) the amino acid at position 20 in the amino acid sequence shown in SEQ ID NO: 1 or at a position corresponding to this position, is substituted with alanine, arginine, asparagine, aspartate, cysteine, glutamine, glutamate, histidine, isoleucine, leucine, lysine, methionine, phenylalanine, proline, serine, threonine, tryptophan, tyrosine, or valine, preferably with arginine, histidine, lysine, or tyrosine, more preferably with arginine,
      preferably wherein the amino acid that is substituted is glycine; and/or
   (2) the amino acid at position 100 in the amino acid sequence shown in SEQ ID NO: 1 or at a position corresponding to this position, is substituted with alanine, arginine, asparagine, aspartate, cysteine, glutamine, glutamate, glycine, histidine, isoleucine, leucine, lysine, methionine, phenylalanine, proline, threonine, tryptophan, tyrosine, or valine, preferably with asparagine,
      preferably wherein the amino acid that is substituted is serine.
3. The GCC of item 1 or 2, wherein the improved activity is improved energetic efficiency.
4. The GCC of item 3, wherein the improved energetic efficiency is reduced ATP consumption, preferably reduced by at least 50%.
5. The GCC of any one of items 1 to 4, wherein the GCC has an ATP/CO₂ ratio of lower than about 4.00±0.02.
6. The GCC of any one of items 1 to 5, wherein the GCC has an ATP/CO₂ ratio of about 1.71±0.11.
7. The GCC of any one of items 1 to 6, wherein the improved activity is increase in carboxylation activity, preferably an at least 2- or 3-fold increase.
8. The GCC of item 7, wherein the increase in carboxylation activity is an increase in conversion of glycolyl-CoA into tartronyl-CoA.
9. The GCC of any one of items 1 to 8, wherein the GCC has a catalytic rate of carboxylation of glycolyl-CoA (conversion of glycolyl-CoA into tartronyl-CoA) of higher than about 5.6±0.3 s⁻¹.
10. The GCC of any one of items 1 to 9, wherein the specific activity of the GCC is higher than about 937±40 nmol glycolyl-CoA min⁻¹ mg⁻¹.
11. The GCC of item 10, wherein the specific activity of the GCC is about 2602±430 nmol glycolyl-CoA min⁻¹ mg⁻¹.
12. A nucleic acid molecule encoding the GCC of any one of items 1 to 11.
13. A vector comprising the nucleic acid molecule of item 12.
14. An organelle comprising the nucleic acid molecule of item 12 or the vector of item 13.
15. A host cell comprising the nucleic acid molecule of item 12, the vector of item 13, or the organelle of item 14.
16. A tissue comprising the host cell of item 15.
17. An organism comprising the nucleic acid molecule of item 12, the vector of item 13 or the organelle of item 14.
18. The organism of item 17, wherein the organism is a plant, algae, or a microorganism.
19. The organism of item 18, wherein the microorganism is a bacterium.
20. The organelle of item 14, the host cell of item 15, the tissue of item 16, or the organism of any one of items 17 to 19, wherein said organelle, said host cell, said tissue, or said organism has a higher conversion rate of glycolyl-CoA into tartronyl-CoA than the corresponding organelle, host cell, tissue, or organism comprising a reference GCC, preferably the reference GCC comprising SEQ ID NO: 1.
21. The organelle of item 14, the host cell of item 15, the tissue of item 16, or the organism of any one of items 17 to 19, wherein said organelle, said host cell, said tissue, or said organism has a higher growth rate and/or carbon yield than the corresponding organelle, host cell, tissue, or organism comprising a reference GCC, preferably the reference GCC comprising SEQ ID NO: 1.
22. Use of the GCC of any one of items 1 to 11, the organelle of item 14, the host cell of item 15, the tissue of item 16, or the organism of any one of items 17 to 19 for CO₂-fixation and/or for a photosynthetic process.
23. Use of the GCC of any one of items 1 to 11, the organelle of item 14, the host cell of item 15, the tissue of item 16, or the organism of any one of items 17 to 19 for decomposition of a synthetic material.
24. Use of item 23, wherein the synthetic material is a polyester.
25. Use of item 24, wherein the polyester is polyethylene terephthalate.
26. Use of the GCC of any one of items 1 to 11, the organelle of item 14, the host cell of item 15, the tissue of item 16, or the organism of any one of items 17 to 19 for decomposition of ethylene glycol and/or glycolate.
27. A method for producing biomass using the GCC of any one of items 1 to 11, the organelle of item 14, the host cell of item 15, the tissue of item 16, or the organism of any one of items 17 to 19.
28. A method for decomposition of a synthetic material using the GCC of any one of items 1 to 11, the organelle of item 14, the host cell of item 15, the tissue of item 16, or the organism of any one of items 17 to 19.
29. The method of item 28, wherein the synthetic material is a polyester.
30. The method of item 29, wherein the polyester is polyethylene terephthalate.
31. A method for decomposition of ethylene glycol and/or glycolate using the GCC of any one of items 1 to 11, the organelle of item 14, the host cell of item 15, the tissue of item 16, or the organism of any one of items 17 to 19.
32. A composition comprising the GCC of any one of items 1 to 11, the nucleic acid of item 12, the vector of item 13, the organelle of item 14, the host cell of item 15, the tissue of item 16, or the organism of any one of items 17 to 19.

The conversion of the C₂ compounds glycolate and glyoxylate into C₃ metabolites plays a central role in many carbon metabolic processes. However, there are only very few natural metabolic routes that allow the direct conversion of these C₂ intermediates into C₃ metabolites, and all of them result in the loss of carbon. The glyoxylate cycle (Kornberg, Nature 179, 988-991, 1957) and the recently described β-hydroxyaspartate cycle (Borzyskowski, Nature 575, 500-504, 2019) arrive at C₄ compounds, which need to be decarboxylated to generate C₃ metabolites. Similarly, photorespiration and the glycerate pathway convert two glyoxylate molecules into glycerate through the release of CO₂ (Krakow, J. Bacteriol. 81, 509-518, 1961; Bauwe, Trends Plant Sci. 15, 330-336, 2010). The inevitable loss of CO₂ in all of these pathways strongly limits their carbon efficiency, which is especially apparent for photorespiration. It was estimated that in hot and dry climates, agricultural crop yield is diminished by up to 50% due to photorespiratory carbon losses (Walker, Annu. Rev. Plant Biol. 67, 107-129, 2016). Thus, circumventing energy and carbon loss during glycolate assimilation through synthetic pathways is expected to enhance productivity substantially. Recently, the tartronyl-CoA pathway was proposed as a direct route for the assimilation of glycolate into central carbon metabolism (Trudeau, Proc. Natl Acad. Sci. USA 115, E11455-E11464, 2018). This previously hypothetical pathway was designed to fix CO₂ instead of releasing it, and is expected to outperform all naturally evolved glycolate assimilation routes. However, the pathway remained theoretical until the present inventors developed in previous work the glycolyl-CoA carboxylase (GCC)—the key enzyme of the tartronyl-CoA pathway. To develop said GCC, the inventors previously introduced five mutations to convert a naturally occurring propionyl-CoA carboxylase (PCC) of Methylorubrum extorquens into GCC M5 that allows the carboxylation of glycolyl-CoA.

The PCC of Methylorubrum extorquens is a two-subunit protein comprising an α-subunit (Protein: SEQ ID NO: 13; DNA: SEQ ID NO: 26) and a β-subunit (Protein: SEQ ID NO: 12; DNA: SEQ ID NO: 25). It is evident for the skilled person that the “actual wildtype” variant/version of the herein described variants of GCC is PCC as can be found in nature in Methylorubrum extorquens. Accordingly, the “actual wildtype” sequence would be the sequence of PCC of Methylorubrum extorquens (α-subunit (Protein: SEQ ID NO: 13; DNA: SEQ ID NO: 26) and a β-subunit (Protein: SEQ ID NO: 12; DNA: SEQ ID NO: 25)).

However, it is further evident for the skilled person that depending on the context also GCC M5 may be referred to as wildtype or reference for the herein described variants. For the herein described inventive variants of GCC, GCC M5 can be considered as the wildtype or reference version because the present inventors started from GCC M5 to optimize GCC. The described amino acid substitutions of the present invention are located in the β-subunit. Thus, it will be mainly referred to herein to the sequences that are or encode the β-subunit of PCC or GCC. The β-subunit of GCC M5 has the amino acid sequence of SEQ ID NO: 1 (the corresponding nucleotide sequence is shown in SEQ ID NO: 14). All amino acid changes of GCC M5 relative to PCC (the “actual wildtype”) are located in the 3-subunit. Accordingly, GCC M5 α-subunit is the α-subunit of the PCC (the “actual wildtype”; (Protein: SEQ ID NO: 13; DNA: SEQ ID NO: 26). All amino acid changes of the inventive GCCs described herein relative to GCC M5 (and relative to PCC (the “actual wildtype”)) are also in the 3-subunit. Accordingly, it is envisaged that the inventive GCCs (inventive variants of GCCs) described herein have/comprise the α-subunit of the PCC (the “actual wildtype”; (Protein: SEQ ID NO: 13; DNA: SEQ ID NO: 26). Accordingly, all disclosures herein where only the β-subunit is defined can be combined with an α-subunit, preferably an α-subunit that consists of or comprises SEQ ID NO: 13. In the context of the present invention, it is understood by the skilled person that the herein provided GCC (i.e., a protein complex having/comprising GCC enzymatic activity) comprises an herein provided inventive GCC β-subunit (e.g., comprising the G20R and/or the S100N amino acid substitutions) and a suitable α-subunit (e.g., the α-subunit of the PCC). As mentioned above, the skilled person is aware that carboxylases (i.e., enzymes that catalyze the carboxylation of one or more substrates) comprise two subunits (i.e., the α- and the β-subunit), whereas each subunit carries their own active site. In particular, in biotin-dependent carboxylases, the α-subunit has biotin carboxylase activity (i.e., cleaves ATP and carboxylates biotin), whereas the β-subunit has carboxyltransferase activity (i.e., catalyzes the trans-carboxylation from biotin to an acceptor molecule, such as glycolyl-CoA). While the substrate specificity of carboxylases is primarily determined by the β-subunit, the α-subunit may be largely conserved between various carboxylases (having different substrate specificities) For example, it was previously shown that α-subunits and β-subunits of different carboxylases from the same organism or even different organisms can form a functional (chimeric) complex (Huang, et al., 2010, Nature, 466(7309):1001-1005, and Lombard and Moreira, 2011, BMC Evol Biol 11, 232, respectively). Accordingly, in the context of the present invention, it is envisaged that the herein provided GCC comprises the inventive β-subunits (such as the β-subunit comprising a G20R and/or a S100N substitution) and further comprises an α-subunit. The nature of said α-subunit is not particularly limited as long as it has biotin carboxylase activity and can form a catalytically active complex with the herein provided β-subunit(s). The skilled person is further aware that such α-subunits may themselves comprise two subunits that collectively form an active enzyme(-complex) having biotin carboxylase activity (see, for example, Cronan and Waldrop, 2002, Progress in Lipid Research, 41, 407-435). Exemplary biotin carboxylases comprising two subunits (also collectively referred to as α-subunit herein) are: Acetyl-CoA carboxylase from E. coli and most other bacterial acetyl-CoA carboxylases, where the α-subunit is separated into two subunits (i.e., where the biotin carboxylase domain and the biotin-carboxyl-carrier-protein domain are separated into two subunits; Family 1.1 in Tong, 2013, Cell. Mol. Life Sci. 70, 863-891; see also Cronan, 2021, Microbiol Mol Biol Rev 85:10). Accordingly, as used herein, the term “α-subunit” in the context of the present invention generally refers to an enzyme(-complex) having biotin carboxylase activity. Exemplary biotin carboxylases comprising one subunit (i.e., an α-subunit) are: Propionyl-CoA carboxylase from H. sapiens and most other propionyl-CoA carboxylases (Family 1.5 in Tong, 2013loc. cit.; see also Huang, 2010, loc. cit.). Accordingly, in the context of the present invention, the α-subunit (i.e., the biotin carboxylase) may optionally be selected from biotin carboxylases as listed herein above. Preferably, however, the α-subunit that forms a complex with the herein provided β-subunit(s) is the α-subunit of the PCC (or an enzymatically active variant thereof).

Accordingly, the present invention relates to a Glycolyl-CoA Carboxylase (GCC), wherein said GCC is characterized in that:
it comprises an amino acid sequence having at least 60% sequence identity to SEQ ID NO:1, and
it has one or more amino acid substitutions, deletions or insertions at a position selected from the group consisting of positions 20 and 100 in the amino acid sequence shown in SEQ ID NO: 1 or at a position corresponding to any of these positions, and
it (further) comprises an α-subunit, preferably wherein said α-subunit is a biotin carboxylase or has biotin carboxylase activity, more preferably wherein said α-subunit comprises/has at least 60% sequence identity to SEQ ID NO: 13, more preferably wherein said α-subunit comprises/has at least 60%, 70%, 80%, 90%, 95%, 96%, 97%, 98%, or 99% sequence identity to SEQ ID NO: 13, even more preferably wherein said α-subunit comprises/has about 100% sequence identity to SEQ ID NO: 13, and
preferably wherein the GCC has an improved activity over a reference GCC, preferably wherein the reference GCC comprises the amino acid sequence of SEQ ID NO:1 and optionally the amino acid sequence of SEQ ID NO: 13.

Accordingly, the present invention relates to a Glycolyl-CoA Carboxylase (GCC), wherein said GCC is characterized in that:
it comprises a β-subunit, wherein said β-subunit:
   comprises/has an amino acid sequence having at least 60% sequence identity to SEQ ID NO:1, and
   has one or more amino acid substitutions, deletions or insertions at a position selected from the group consisting of positions 20 and 100 in the amino acid sequence shown in SEQ ID NO: 1 or at a position corresponding to any of these positions,
and
said GCC (further) comprises an α-subunit, preferably wherein said α-subunit is a biotin carboxylase or has biotin carboxylase activity, more preferably wherein said α-subunit comprises/has at least 60% sequence identity to SEQ ID NO: 13, more preferably wherein said α-subunit comprises/has at least 60%, 70%, 80%, 90%, 95%, 96%, 97%, 98%, or 99% sequence identity to SEQ ID NO: 13, even more preferably wherein said α-subunit comprises/has about 100% sequence identity to SEQ ID NO: 13, and
preferably wherein the GCC has an improved activity over a reference GCC, preferably wherein the reference GCC comprises the amino acid sequence of SEQ ID NO:1 and optionally the amino acid sequence of SEQ ID NO: 13.

In this context, the β-subunit(s) and α-subunit(s) may be as defined as anywhere herein above or below.

In the context of the present invention, and as has been illustratively demonstrated in the enclosed examples and FIG. 6, the herein provided GCCs can comprise 6 β-subunits and 6 α-subunit (i.e., can form a β₆α₆ complex). However, complexes comprising less than 6 α-subunits may also be envisaged in the context of the present application. Accordingly, the herein provided GCC may comprise 6 β-subunits and 1 to 6 α-subunits, preferably 6 α-subunits. This is illustrated in FIG. 6.

Accordingly, the present invention relates to a Glycolyl-CoA Carboxylase (GCC), wherein said GCC may be characterized in that:
it comprises six β-subunit, wherein each of said β-subunits:
   comprises/has an amino acid sequence having at least 60% sequence identity to SEQ ID NO:1, and
   has one or more amino acid substitutions, deletions or insertions at a position selected from the group consisting of positions 20 and 100 in the amino acid sequence shown in SEQ ID NO: 1 or at a position corresponding to any of these positions, and
said GCC (further) comprises one to six α-subunit(s), preferably wherein each of said α-subunit(s) is a biotin carboxylase or has biotin carboxylase activity, more preferably wherein each of said α-subunit(s) comprises/has at least 60% sequence identity to SEQ ID NO: 13, more preferably wherein each of said α-subunits comprises/has at least 60%, 70%, 80%, 90%, 95%, 96%, 97%, 98%, or 99% sequence identity to SEQ ID NO: 13, even more preferably wherein each of said α-subunits comprises/has about 100% sequence identity to SEQ ID NO: 13, and
preferably wherein the GCC has an improved activity over a reference GCC, preferably wherein the reference GCC comprises the amino acid sequence of SEQ ID NO:1 and optionally the amino acid sequence of SEQ ID NO: 13.

In this context, the β-subunit(s) and α-subunit(s) may be as defined as anywhere herein above or below.

It is further envisaged herein, that the herein provided GCC comprises one or more β-subunit(s) and α-subunit(s) (each) having/comprising varying amino acid sequences, however, it is herein preferred that the herein provided GCC comprises only one type of β-subunit and α-subunit (i.e., it is preferred that all β-subunits comprise the same amino acid sequence and that all α-subunits comprise the same amino acid sequence).

Accordingly, the amino acid substitutions can be denoted relative to the “actual wildtype” sequence which would be the β-subunit of PCC (Protein: SEQ ID NO: 12; DNA: SEQ ID NO: 25). However, the amino acid substitutions may also be denoted relative to the GCC M5 sequence, i.e. relative to SEQ ID NO: 1. FIG. 3 shows an alignment of PCC with GCC M5 and two GCCs of the present invention. To develop GCC M5 the inventors introduced five mutations into PCC (L100S, Y143H, D407I, I450V and W502R). Thus, GCC M5 has a serine at position 100. However, when it is referred herein to an amino acid substitution of amino acid position 100 in GCC M5 (SEQ ID NO: 1) the substitution may nevertheless be denoted relative to the “actual wildtype” sequence. For example, when it is referred herein to the amino acid substitution of position 100 of GCC M5 to e.g., arginine, said substitution may be denoted as L100N. In this case the substitution is denoted relative to the “actual wildtype” sequence. However, it is evident for the skilled person that amino acid substitution relative to GCC M5 (and thus relative to SEQ ID NO: 1) is S100N. In other words, it is evident for the skilled person that when the amino acid at position 100 in GCC M5 is replaced by arginine a serine is replaced by said arginine.

Since GCC M5 is the enzyme of the prior art, SEQ ID NO: 1 is used as the reference sequence for describing the variants of the invention. Accordingly, the invention provides positions in GCC M5 (SEQ ID NO: 1) for which amino acid substitutions improve activity. In other words, the inventors identified amino acid positions which substitution results in improved activity of the GCC. In other words, the present inventors identified amino acid residues in GCC M5, which can be substituted with other amino acid residues to alter activity of GCC M5 (as observed from altered energetic efficiency and/or carboxylation activity as described elsewhere herein). The terms “amino acid” and “amino acid residue” may be used synonymously herein. The terms “substituted”, “replaced” and “mutated” may be used synonymously herein. Accordingly, also the terms “substitution”, “replacement” and “mutation” may be used synonymously herein. An example for a substitution would be that the glycine at position 20 in SEQ ID NO: 1 or at a position corresponding to this position is substituted by arginine. Another example for a substitution would be that the serine at position 100 in SEQ ID NO: 1 or at a position corresponding to this position is substituted by asparagine. The skilled person is well aware how amino acids can be substituted by molecular biological techniques, such as site-directed mutagenesis as described herein. It is also envisaged that at the herein described positions amino acids are inserted and/or deleted. In the context of the present invention, “deleted” or “deletion” means that the amino acid at the corresponding position is deleted or removed.

In the context of the present invention, “inserted” or “insertion” means that at the respective position one or more, preferably one amino acid residue is inserted.

It is pointed out that the GCCs of the invention can also be referred to as GCC variants of the invention. Accordingly, “GCC” and “GCC variant” may be used synonymously. Also “GCC mutant variant” or “GCC version” may be used.

The skilled person is well aware how substitutions, insertions or deletions may be introduced. Site-directed mutagenesis as described herein below may be used to generate the herein described GCCs. Furthermore, a random mutagenesis library may be generated to produce a dataset to feed an artificial intelligence (AI) algorithm. The random mutagenesis library may be generated as described in the following.

Plasmid libraries of randomly mutagenized GCC M5 may be created by mega primer-based whole-plasmid PCR (MEGAWHOP) (Miyazaki, Methods Enzymol. 498, 399-406, 2011). To generate randomized fragments of e.g. the β subunit of GCC M5 (pTE3101), error-prone PCR may be performed using 2.5 U Taq-polymerase with Mg-free buffer (New England Biolabs; M0320), 7 mM MgCl₂, 0.4 mM dGTP and dATP each, 2 mM dCTP and dTTP each, 0.4 μM primer PccB_fw_P1 and primer PccB_rv_P1 each, 10% (v/v) dimethyl sulfoxide, 50 ng template DNA of pTE3101, and 200-500 μM MnCl₂ in a 50 μL reaction. The randomized fragments may be digested with DpnI (NEB, R0176), purified by agarose gel electrophoresis, and used as mega primers for a whole-plasmid PCR (MEGAWHOP), as described elsewhere (Miyazaki, loc. cit), or subjected to another error-prone PCR reaction to further increase the mutation rate. The MEGAWHOP reaction (50 μL) may contain 1×KOD Hot Start reaction buffer (Novagen), 0.2 mM dNTPs, 1.5 mM MgSO₄, 500 ng mega primer, 50 ng template plasmid (GCC M5; pTE3101), and 2.5 U KOD Hot Start DNA polymerase (Novagen). The MEGAWHOP product may be purified, digested with DpnI, and transformed into ElectroMAX DH5a (Thermo Fisher Scientific) to ensure a high number of transformants in the resulting libraries. To estimate the mutation rate for the different concentrations of MnCl₂ used in the error-prone PCR, the plasmids of ten randomly picked clones after MEGAWHOP may be purified, sequenced and analyzed for nucleotide exchanges.

In order to develop (additional) GCC variants with improved kinetic properties an artificial intelligence (AI) algorithm may be used. A learning dataset to train the AI model may be produced by generating a random mutagenesis library of pTE3101 (GCC M5), transforming it into chemically competent E. coli BL21-birA and colony picking into 96-deep-well plates (PlateOne) with lysogeny broth (Miller) containing 100 μg/mL ampicillin and 50 μg/mL spectinomycin. Expression, lysis and screening of GCC samples may be done as described earlier (Scheffen loc. cit.). 2,100 mutant variants may be screened and from the obtained data a subset of representative candidates may be selected for sequencing. 161 samples may be used to train the machine-learning model from Exazyme towards the prediction of beneficial mutations in GCC M5. The resulting list of all possible single mutations ranked by their efficiency may be used as a template for identifying suitable candidates for biochemical characterization by homology model creation and structural investigation of promising mutations.

In order to assess mutations that are predicted by the AI algorithm, homology modelling of each promising mutant variant may be performed using SWISS-MODEL. As a template for homology modelling of GCC mutations, the structure of the engineered GCC M5 from Methylobacterium extorquens (PDB ID 6YBQ) may be used. Structural analysis of the models may be done using PyMOL (the PyMOL Molecular Graphics System; version 1.8; Schrödinger). Modelling of glycolyl-CoA into the active site of GCC, respectively, may be based on the positions of CoA in the GCC M5 structure and methylmalonyl-CoA in the structure of a methylmalonyl-CoA carboxytransferase from Propionibacterium freudenreichii (PDB ID 1ON3; 52% amino acid identity). Manual fitting and adjustments of the CoA thioesters reflecting differences in active-site architectures may be done with COOT and PyMOL.

Site-directed mutagenesis may be used to construct mutant variants of GCC that were earlier predicted by the AI algorithm and selected by homology modelling and structural analysis. The introduction of novel mutations may be done by single mutagenic oligonucleotide PCR as described elsewhere (Shenoy Anal. Biochem. 319, 335-336, 2003). Of course, these techniques can also be used for the herein described GCCs. A 25 μL reaction mixture containing 0.5 μM primer, 3% (v/v) dimethyl sulfoxide, 50 ng template DNA (pTE3101) and Phusion High-Fidelity PCR Master Mix (NEB, M0531) may be used for PCR and subsequently digested with DpnI (NEB, R0176) by adding 20 U to the reaction mixture and incubating 2 h at 37° C. 5 μL may be transformed into chemically competent E. coli NEB Turbo cells and streaked out on lysogeny broth (Miller) agar plates with 50 μg/mL streptomycin. Three to six colonies may be picked, cultivated in 10 mL lysogeny broth (Miller) with 50 μg/mL streptomycin for 12 h at 37° C. and 180 rpm and finally the plasmids may be isolated and sequenced to validate the mutagenesis. For mutagenesis and sequencing, the primers/oligonucleotides of Table 4 may be used. As bacterial strains for mutagenesis and cloning, the strains of Table 2 may be used. E. coli ElectroMAX DH5a may be used to create random mutagenesis libraries that may be needed to produce a dataset of randomly mutagenized GCC variants to train an artificial intelligence model for the prediction of beneficial mutations. E. coli NEB Turbo may be used to construct and maintain plasmids with site-specific mutations in the gene/nucleic acid for/encoding GCC. E. coli BL21-birA may be derived from E. coli BL21 DE3 by introducing a vector that bears a biotin ligase gene from Methylorubrum extorquens that is required to activate GCC. E. coli BL21-birA may be used for protein overexpression of GCC variants.

The skilled person knows that some carboxylases are biotin-dependent. PCCs for example belong to the family of biotin-dependent carboxylases. Biotin may be covalently bound to a lysine in the PCC α-subunit and may act as a cofactor for the carboxylation reaction. Accordingly, the skilled person knows that also the PCC of Methylorubrum extorquens is biotin-dependent (Scheffen loc. cit.). Accordingly, the skilled person knows that also GCC M5 is biotin-dependent. Thus, it is envisaged that also the GCCs of the invention are biotin-dependent. Accordingly, it is evident for the skilled person that the herein described GCCs may be contacted with a biotin-ligase at some point to become active. In other words, it is envisaged that the herein described GCCs may be contacted with a biotin-ligase at some point to be able to catalyze the carboxylation reaction. For example, a biotin-ligase may be co-expressed/co-produced together with the herein described GCCs. It is preferred that a biotin-ligase of Methylorubrum extorquens is co-expressed/co-produced together with the herein described GCCs. It is also envisaged that a biotin-ligase gene is introduced into the host that is used for production of the herein described GCCs. It is preferred that biotin ligase gene from Methylorubrum extorquens is introduced into the host that is used for production of the herein described GCCs.

Furthermore, it is evident that when a host cell, organelle, tissue and/or organism as described herein comprises the GCCs as described herein it may also comprise a suitable biotin ligase. The suitable biotin-ligase may be an endogenous biotin ligase or a recombinantly introduced biotin-ligase. The skilled person knows that biotinylation of a GCC may be determined e.g. by an avidin gel shift assay (Scheffen loc. cit.). Accordingly, the skilled person can test whether a given biotin ligase can biotinylate a GCC and is, thus, suitable. Additionally, the skilled person can test whether a GCC is fully biotinylated or only partially. In a preferred aspect the host cell, organelle, tissue and/or organism as described herein may comprise biotin ligase gene from Methylorubrum extorquens. In other words, in a preferred aspect the host cell, organelle, tissue and/or organism as described herein may produce a biotin ligase from Methylorubrum extorquens (in addition to the herein described GCCs). The biotin ligase from Methylorubrum extorquens has an amino acid sequence as shown in SEQ ID NO: 27 and/or a nucleic acid sequence as shown in SEQ ID NO: 28.

Chemicals for the herein described techniques may be obtained from Sigma-Aldrich, Carl Roth GmbH+Co. KG, Santa Cruz Biotechnology Inc. and Merck. Biochemicals and materials for cloning and protein expression may be obtained from Thermo Fisher Scientific, New England Biolabs GmbH and Macherey-Nagel GmbH. Coenzyme A may be bought from Roche Diagnostics. Materials and equipment for protein purification may be obtained from GE Healthcare, BioRad and Merck Millipore GmbH. Pyruvate Kinase/Lactic Dehydrogenase, Malic Dehydrogenase, Glucose-6-Phosphate Dehydrogenase, Glucose Dehydrogenase and Phosphoenolpyruvate carboxylase may be bought from Sigma-Aldrich.

The invention provides a GCC, wherein said GCC is characterized in that:
it comprises an amino acid sequence having at least 60% sequence identity to SEQ ID NO: 1, and
it has one or more amino acid substitutions, deletions or insertions at a position selected from the group consisting of positions 20 and 100 in the amino acid sequence shown in SEQ ID NO: 1 or at a position corresponding to any of these positions.

It is envisaged that the herein described GCCs have improved activity e.g. over a reference GCC that does not comprise the herein described amino acid substitutions, deletions, or insertions.

The invention provides a GCC, wherein said GCC is characterized in that:
it comprises an amino acid sequence having at least 60% sequence identity to SEQ ID NO: 1, and
it has one or more amino acid substitutions, deletions or insertions at a position selected from the group consisting of positions 20 and 100 in the amino acid sequence shown in SEQ ID NO: 1 or at a position corresponding to any of these positions, wherein the GCC has an improved activity over a reference GCC.

It is envisaged that the herein described GCCs (e.g. the GCCs with the inventive amino acid substitutions) have improved activity over a reference GCC comprising SEQ ID NO: 1.

The invention provides a Glycolyl-CoA Carboxylase (GCC), wherein said GCC is characterized in that:
it comprises an amino acid sequence having at least 60% sequence identity to SEQ ID NO: 1, and
it has one or more amino acid substitutions, deletions or insertions at a position selected from the group consisting of positions 20 and 100 in the amino acid sequence shown in SEQ ID NO: 1 or at a position corresponding to any of these positions,
wherein the GCC has an improved activity over a reference GCC comprising the amino acid sequence of SEQ ID NO: 1.

As mentioned above, it is envisaged herein that a GCC comprises two subunits (for example a GCC variant provided herein comprises an α-subunit and a β-subunit). Accordingly, it is evident for the skilled person that the reference GCC comprises also two subunits (α-subunit and β-subunit). Preferably, the β-subunit of the reference GCC consists of or comprises SEQ ID NO: 1. Preferably, the α-subunit of the reference GCC consists of or comprises SEQ ID NO: 13. Accordingly, it is preferred that the activity of the GCCs of the invention are compared to the activity of a reference GCC, wherein said reference GCC comprises an α-subunit that consists of or comprises SEQ ID NO: 13 and a β-subunit that consists of or comprises SEQ ID NO: 1. As explained above the prior art enzyme GCC M5 consists of an α-subunit that consists of SEQ ID NO: 13 and a β-subunit that consists of SEQ ID NO: 1. Accordingly, it is preferred that the activity of the GCCs of the invention are compared to the activity of a reference GCC, wherein said reference GCC is GCC M5.

In context of the present invention, improved activity of the GCC or improved enzymatic activity of the GCC may refer to improved energetic efficiency and/or improved carboxylation activity. Preferably, GCC variants having improved energetic efficiency are variants having a, amino acid substitution, deletion or insertion at position 100 as defined herein.

Improved energetic efficiency may mean herein that less energy input is required to perform a certain reaction. In other words, improved energetic efficiency may mean that less energy equivalents are required for GCC to perform a certain reaction. In other words, improved energetic efficiency may mean that the GCCs of the invention require less energy input/less energy equivalents (i.e., ATP) to convert glycolyl-CoA into tartronyl-CoA/for the conversion of glycolyl-CoA into tartronyl-CoA as compared to a reference GCC, preferably a reference GCC comprising SEQ ID NO: 1. The term “tartronyl-CoA” as used herein refers particularly to “(S)-tartronyl-CoA”.

Accordingly, it is envisaged that the improved activity of the herein described GCCs is improved energetic efficiency. In other words, the present invention provides GCCs with improved activity, wherein the improved activity is improved energetic efficiency.

Improved energetic efficiency in context of the invention may herein refer to reduced ATP consumption. ATP is a widely known energy equivalent of biological systems. Thus, improved energetic efficiency as used herein may mean that less ATP is required for the herein described GCCs to convert glycolyl-CoA into tartronyl-CoA/for the conversion of glycolyl-CoA into tartronyl-CoA as compared to a reference GCC, preferably a reference GCC comprising SEQ ID NO: 1. Accordingly, it is envisaged that the improved energetic efficiency of the herein described GCCs is reduced ATP consumption. In other words, the present invention provides GCCs with improved energetic efficiency, wherein the improved energetic efficiency is reduced ATP consumption.

It is envisaged that the ATP consumption is reduced by at least 5%, at least 10%, at least 20%, at least 30%, at least 40%, at least 50%, at least 60%, at least 70%, at least 80%, at least 90%, or at least 95%, preferably by at least 50%. Accordingly, the present invention provides GCCs with at least 50% reduced ATP consumption compared to a reference GCC, preferably a reference GCC comprising SEQ ID NO: 1.

GCC converts glycolyl-CoA to tartronyl-CoA by addition of CO₂ under consumption of ATP (glycolyl-CoA+HCO₃⁻+ATP→tartronyl-CoA+ADP+Pᵢ). As mentioned above, GCC M5 has an ATP per carboxylation ratio of about 4 although the theoretical ratio is 1. That is, the theoretical ratio of consumed ATP molecule for one added CO₂ molecule is 1. However, the measured amount of consumed ATP is about 4 molecules for one added CO₂ molecule. As also demonstrated in the appended examples an ATP/CO₂ ratio of the described GCCs can be determined. The ATP/CO₂ ratio of any GCC variant can be readily determined by the skilled person by first assessing the ATP consumption and carboxylation rate of the given GCC variant and then calculating the ATP/CO₂ ratio. An ATP/CO₂ ratio of 4 means that four molecules ATP are consumed for fixation of one molecule CO₂ (i.e., for the conversion of one molecule glycolyl-CoA to tartronyl-CoA).

Accordingly, it is envisaged that the herein described GCCs have/show an ATP/CO₂ ratio of lower than about 4.00±0.02. Thus, the invention provides a GCC, wherein the GCC has an ATP/CO₂ ratio of lower than about 4.00±0.02. In other words, the improved activity of the herein described GCCs and/or the improved energetic efficiency of the herein described GCCs may be evident from the fact that said GCCs have an ATP/CO₂ ratio of lower than about 4.00±0.02. It is evident from the appended examples that GCC M5 has an ATP/CO₂ ratio of 4.00±0.02. Accordingly, the GCCs of the present invention have an improved energetic efficiency compared to a reference GCC (e.g. GCC M5) when they have an ATP/CO₂ ratio of lower than about 4.00±0.02. It is also envisaged that the herein described GCCs have an ATP/CO₂ ratio of lower than about 4, lower than about 3, lower than about 2, or lower than about 1, or any value in between such as 2.5.

Thus, it is envisaged that the herein described GCCs have an ATP/CO₂ ratio of about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8 or about 3.9 or any value in between such as 2.23. It is preferred that the described GCC has an ATP/CO₂ ratio of about 1.71±0.11. Thus, the present invention provides a GCC, wherein the GCC has an ATP/CO₂ ratio of about 1.71±0.11. A GCC of the invention comprising SEQ ID NO: 7 may have an ATP/CO₂ ratio of about 1.71±0.11. In a preferred aspect, the present invention relates to a GCC comprising SEQ ID NO: 7, wherein the GCC has an ATP/CO₂ ratio of about 1.71±0.11.

The explanations herein in relation to “improved energetic efficiency”, “reduced ATP consumption”, “ATP/CO₂ ratio of lower than about 4.00±0.02”, “an ATP/CO₂ ratio of about 1.71±0.11” and the like particularly apply to a Glycolyl-CoA Carboxylase (GCC), wherein said GCC is characterized in that it comprises an amino acid sequence having at least 60% sequence identity to SEQ ID NO:1, and it has (only) one amino acid substitution, deletion or insertion (preferably substitution) at position 100 in the amino acid sequence shown in SEQ ID NO: 1 or at a position corresponding to this position, preferably an asparagine at position 100. “Only” in this context means that the GCC does not have an amino acid substitution, deletion or insertion (preferably substitution) at position 20 in the amino acid sequence shown in SEQ ID NO: 1 or at a position corresponding to this position. In this context, it is preferred that the GCC only has one amino acid substitution, deletion or insertion (preferably substitution) at position 100 in the amino acid sequence shown in SEQ ID NO: 1 or at a position corresponding to any of these positions, i.e. does not have an amino acid substitution, deletion or insertion (preferably substitution) at position 20 as defined above.

Thus, in one aspect, the present invention relates to a Glycolyl-CoA Carboxylase (GCC), wherein said GCC is characterized in that:
it comprises an amino acid sequence having at least 60% sequence identity to SEQ ID NO:1, and
it has (only) one amino acid substitution, deletion or insertion at position 100 in the amino acid sequence shown in SEQ ID NO: 1 or at a position corresponding to any of these positions, preferably wherein the GCC has an improved activity over a reference GCC, preferably wherein the reference GCC comprises the amino acid sequence of SEQ ID NO:1, preferably wherein the GCC has improved energetic efficiency as defined herein.

The explanations herein in relation to “increase in carboxylation activity”, “increase in conversion of glycolyl-CoA into tartronyl-CoA”, “catalytic rate of carboxylation of glycolyl-CoA (conversion of glycolyl-CoA into tartronyl-CoA) of higher than about 5.6±0.3 s⁻¹”, “specific activity of the GCC is higher than about 937±40 nmol glycolyl-CoA min⁻¹ mg⁻¹”, “specific activity of the GCC is about 2602±430 nmol glycolyl-CoA min⁻¹ mg⁻¹” and the like particularly apply to a Glycolyl-CoA Carboxylase (GCC), wherein said GCC is characterized in that it comprises an amino acid sequence having at least 60% sequence identity to SEQ ID NO:1, and it has (only) one amino acid substitution, deletion or insertion (preferably substitution) at position 20 in the amino acid sequence shown in SEQ ID NO: 1 or at a position corresponding to this position, preferably an arginine at position 20. “Only” in this context means that the GCC does not have an amino acid substitution, deletion or insertion (preferably substitution) at position 100 in the amino acid sequence shown in SEQ ID NO: 1 or at a position corresponding to this position. In this context, it is preferred that the GCC only has one amino acid substitution, deletion or insertion (preferably substitution) at position 20 in the amino acid sequence shown in SEQ ID NO: 1 or at a position corresponding to any of these positions, i.e. does not have an amino acid substitution, deletion or insertion (preferably substitution) at position 100 as defined herein.

Thus, in one aspect, the present invention relates to a Glycolyl-CoA Carboxylase (GCC), wherein said GCC is characterized in that:
it comprises an amino acid sequence having at least 60% sequence identity to SEQ ID NO:1, and
it has (only) one amino acid substitution, deletion or insertion at position 20 in the amino acid sequence shown in SEQ ID NO: 1 or at a position corresponding to any of these positions, preferably wherein the GCC has an improved activity over a reference GCC, preferably wherein the reference GCC comprises the amino acid sequence of SEQ ID NO:1, preferably wherein the improved activity is increase in carboxylation activity, as defined herein.

In one aspect, the GCC herein does not have amino acid substitutions, deletions or insertions (preferably substitutions) at both positions 20 and 100 as defined herein, e.g. does not have both an arginine at position 20 or an asparagine at position 100 in the amino acid sequence shown in SEQ ID NO: 1 or at a position corresponding to any of these positions.

The skilled person is well aware how ATP consumption and CO₂ fixation may be measured (Scheffen, loc. cit.). Among others, spectrophotometric measurements of ATP hydrolysis may be performed. The ATP/CO₂ ratio is also referred to as “ATP per carboxylation ratio”. The determination of ATP per carboxylation ratios may occur via spectrophotometric measurements with purified enzymes.

For the overexpression of GCC M5 and its mutant variants, the corresponding plasmid may be transformed into chemically competent E. coli BL21-birA cells. Cells may be grown on lysogeny broth (Miller) agar plates containing 100 μg/mL ampicillin and 50 μg/mL spectinomycin at 25° C. overnight. 8 L Golden lysogeny broth (Miller) containing 5 g/L yeast extract, 10 g/L tryptone, 10 g/L NaCl, 17 mM KH₂PO₄, 72 mM K₂HPO₄, and 0.4% glycerol may be inoculated from the agar plate and incubated at 37° C. and 140 rpm. At OD₆₀₀=0.4-0.6 protein expression may be induced with 500 μM IPTG and cells may be incubated over night at 25° C. Cell harvesting at 8,000 g and 4° C. for 12 min and lysis by French pressing may be followed by His-Trap purification using an Åkta Start (GE Healthcare) linked to a HisTrap FF column (GE Healthcare). The purification buffer may contain 50 mM HEPES pH 7.8 and 500 mM KCl, the elution may be done with 500 mM imidazole. Protein desalting may occur via gel filtration chromatography using a HiLoad 16/600 Superdex 200 pg column (GE Healthcare) and a buffer containing 50 mM HEPES pH 7.8 and 150 mM KCl. Protein quantification may occur by absorbance measurement at 280 nm. Protein purity may be validated by SDS-PAGE using 15 μg of purified protein on a 4-20% Mini-Protean TGX Precast Protein Gel (Biorad).

Glycolyl-CoA may be synthesized and purified as previously described (Trudeau, loc. cit.; Scheffen, loc. cit.). The concentration of CoA-esters may be quantified by determining the absorption at 260 nm (ε=16.4 mM⁻¹ cm⁻¹).

To measure the ratio between ATP consumption and carboxylation (ATP/CO₂ ratio) of GCC, a coupled enzyme assay with CaMCR under ATP-limited conditions may be performed. 100 mM MOPS pH 7.8, 50 mM KHCO₃, 0.15 mM ATP, 0.5 mM NADPH, 5 mM MgCl₂, 1.8 mg/mL CaMCR from Chloroflexus aurantiacus, and 0.05-3 mg/mL GCC may be mixed in a cuvette and incubated for 2 min at 37° C. The reaction may be started with 0.5 mM glycolyl-CoA and absorption may be measured over time at λ=340 nm. The ATP per carboxylation ratio is calculated from the ratio between the ATP amount in the reaction mixture and the consumed amount of NADPH that is reflected by the absorbance drop during the reaction.

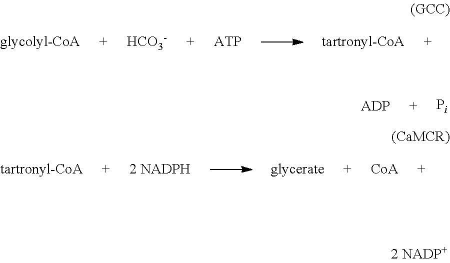

As mentioned already above, in context of the present invention improved activity of the GCC or improved enzymatic activity of the GCC may refer to improved carboxylation activity. Preferably, GCC variants having improved carboxylation activity are variants having an amino acid substitution, deletion or insertion at position 20 as defined herein. Carboxylation is a chemical reaction that may comprise the incorporation of carbon dioxide into an organic compound resulting in the production of a carboxylic acid. “Improved carboxylation activity” as used herein may refer to an increase in conversion of glycolyl-CoA into tartronyl-CoA. Accordingly, it is envisaged that the herein described GCCs have/show an increase in carboxylation activity. It is also envisaged that the herein described GCCs have/show an increase of conversion of glycolyl-CoA into tartronyl-CoA. Thus, the present invention provides GCCs with improved activity, wherein the improved activity is increase in carboxylation activity. The present invention provides GCCs which have/show improved activity, wherein the improved activity is increase in carboxylation activity

The present invention also provides GCCs with increased carboxylation activity, wherein the increase in carboxylation activity is an increase in conversion of glycolyl-CoA into tartronyl-CoA. The present invention also provides GCCs which have/show increased carboxylation activity, wherein the increase in carboxylation activity is an increase in conversion of glycolyl-CoA into tartronyl-CoA.

It is envisaged that the increase in carboxylation activity is at least a 2-, 3-, 4-, 5-, 6-, 7-, 8-, 9- or 10-fold increase. Preferably the increase in carboxylation activity is at least a 2- or 3-fold increase.

The carboxylation activity can be measured as catalytic rate of carboxylation of glycolyl-CoA (conversion of glycolyl-CoA into tartronyl-CoA) (Scheffen, loc. cit.). Catalytic rate (k_{cat}) is here defined as the maximum formation rate of tartronyl-CoA out of glycolyl-CoA and HCO₃⁻. It describes how many mol of the substrate are converted per time and per mol of enzyme. It can only be approximated as it requires 100% substrate saturation of the active sites. Therefore, turnover rates at different initial substrate concentrations can be measured to compute the catalytic rate with the Michaelis-Menten equation. It is envisaged that the catalytic rate of carboxylation of glycolyl-CoA of the herein described GCCs is higher than about 5.6±0.3 s⁻¹.

It is envisaged that the catalytic rate of carboxylation of glycolyl-CoA of the herein described GCCs is higher than about 5.3 s⁻¹, about 5.4 s⁻¹, about 5.5 s⁻¹, about 5.6 s⁻¹, about 5.7 s⁻¹, about 5.8 s⁻¹, about 5.9 s⁻¹, about 6 s⁻¹, about 7 s⁻¹, about 8 s⁻¹, about 9 s⁻¹, about 10 s⁻¹, about 11 s⁻¹, about 12 s⁻¹, about 13 s⁻¹, about 14 s⁻¹, about 15 s⁻¹, about 16 s⁻¹, about 17 s⁻¹, about 18 s⁻¹, about 19 s⁻¹, about 20 s⁻¹, about 21 s⁻¹, about 22 s⁻¹, about 23 s⁻¹, about 24 s⁻¹, about 25 s⁻¹ or any value in between such as 8.3 s⁻¹. It is preferred that the herein described GCCs have a catalytic rate of carboxylation of glycolyl-CoA (conversion of glycolyl-CoA into tartronyl-CoA) of higher than about 5.6±0.3 s⁻¹. In a preferred aspect, the present invention relates to a GCC comprising SEQ ID NO: 3, wherein the GCC has a catalytic rate of carboxylation of glycolyl-CoA (conversion of glycolyl-CoA into tartronyl-CoA) of higher than about 5.6±0.3 s⁻¹.

The carboxylation activity can also be measured as specific activity of the GCC. The specific activity describes how many mol of the substrate are converted per time per amount of enzyme. The maximum specific activity (vₘₐₓ) is proportional to the catalytic rate and can only be approximated as it requires 100% substrate saturation of the active sites. Therefore, specific activities at different initial substrate concentrations can be measured to compute vₘₐₓ. Specifically, herein the specific activity describes how many nmol glycolyl-CoA are converted per minute per mg of GCC. As evident from the appended examples the preferred reference GCC, GCC M5 has a specific activity of about 937±40 nmol glycolyl-CoA min⁻¹ mg⁻¹. Accordingly, it is envisaged that the GCCs of the present invention have a specific activity that is higher than about 937±40 nmol glycolyl-CoA min⁻¹ mg⁻¹. The present invention provides a GCC, wherein the specific activity of the GCC is higher than about 937±40 nmol glycolyl-CoA min⁻¹ mg⁻¹. In a preferred aspect, the present invention relates to a GCC comprising SEQ ID NO: 3, wherein the specific activity of the GCC is higher than about 937±40 nmol glycolyl-CoA min⁻¹ mg⁻¹. It is also envisaged that the herein described GCCs have a specific activity that is higher than about 940 nmol glycolyl-CoA min⁻¹ mg⁻¹, about 950 nmol glycolyl-CoA min⁻¹ mg⁻¹, about 960 nmol glycolyl-CoA min⁻¹ mg⁻¹, about 970 nmol glycolyl-CoA min⁻¹ mg⁻¹, about 980 nmol glycolyl-CoA min⁻¹ mg⁻¹, about 990 nmol glycolyl-CoA min⁻¹ mg⁻¹, about 1000 nmol glycolyl-CoA min⁻¹ mg⁻¹, about 1100 nmol glycolyl-CoA min⁻¹ mg⁻¹, about 1200 nmol glycolyl-CoA min⁻¹ mg⁻¹, about 1300 nmol glycolyl-CoA min⁻¹ mg⁻¹, about 1400 nmol glycolyl-CoA min⁻¹ mg⁻¹, about 1500 nmol glycolyl-CoA min⁻¹ mg⁻¹, about 1600 nmol glycolyl-CoA min⁻¹ mg⁻¹, about 1700 nmol glycolyl-CoA min⁻¹ mg⁻¹, about 1800 nmol glycolyl-CoA min⁻¹ mg⁻¹, about 1900 nmol glycolyl-CoA min⁻¹ mg⁻¹, about 2000 nmol glycolyl-CoA min⁻¹ mg⁻¹, about 2100 nmol glycolyl-CoA min⁻¹ mg⁻¹, about 2200 nmol glycolyl-CoA min⁻¹ mg⁻¹, about 2300 nmol glycolyl-CoA min⁻¹ mg⁻¹, about 2400 nmol glycolyl-CoA min⁻¹ mg⁻¹, about 2500 nmol glycolyl-CoA min⁻¹ mg⁻¹, about 2600 nmol glycolyl-CoA min⁻¹ mg⁻¹, about 2700 nmol glycolyl-CoA min⁻¹ mg⁻¹, about 2800 nmol glycolyl-CoA min⁻¹ mg⁻¹, about 2900 nmol glycolyl-CoA min⁻¹ mg⁻¹, about 3000 nmol glycolyl-CoA min⁻¹ mg⁻¹ or any value in between such as 2339 nmol glycolyl-CoA min⁻¹ mg⁻¹.

It is also envisaged that the herein described GCCs have a specific activity of about 940 nmol glycolyl-CoA min⁻¹ mg⁻¹, about 950 nmol glycolyl-CoA min⁻¹ mg⁻¹, about 960 nmol glycolyl-CoA min⁻¹ mg⁻¹, about 970 nmol glycolyl-CoA min⁻¹ mg⁻¹, about 980 nmol glycolyl-CoA min⁻¹ mg⁻¹, about 990 nmol glycolyl-CoA min⁻¹ mg⁻¹, about 1000 nmol glycolyl-CoA min⁻¹ mg⁻¹, about 1100 nmol glycolyl-CoA min⁻¹ mg⁻¹, about 1200 nmol glycolyl-CoA min⁻¹ mg⁻¹, about 1300 nmol glycolyl-CoA min⁻¹ mg⁻¹, about 1400 nmol glycolyl-CoA min⁻¹ mg⁻¹, about 1500 nmol glycolyl-CoA min⁻¹ mg⁻¹, about 1600 nmol glycolyl-CoA min⁻¹ mg⁻¹, about 1700 nmol glycolyl-CoA min⁻¹ mg⁻¹, about 1800 nmol glycolyl-CoA min⁻¹ mg⁻¹, about 1900 nmol glycolyl-CoA min⁻¹ mg⁻¹, about 2000 nmol glycolyl-CoA min⁻¹ mg⁻¹, about 2100 nmol glycolyl-CoA min⁻¹ mg⁻¹, about 2200 nmol glycolyl-CoA min⁻¹ mg⁻¹, about 2300 nmol glycolyl-CoA min⁻¹ mg⁻¹, about 2400 nmol glycolyl-CoA min⁻¹ mg⁻¹, about 2500 nmol glycolyl-CoA min⁻¹ mg⁻¹, about 2600 nmol glycolyl-CoA min⁻¹ mg⁻¹, about 2700 nmol glycolyl-CoA min⁻¹ mg⁻¹, about 2800 nmol glycolyl-CoA min⁻¹ mg⁻¹, about 2900 nmol glycolyl-CoA min⁻¹ mg⁻¹, about 3000 nmol glycolyl-CoA min⁻¹ mg⁻¹ or any value in between such as 2339 nmol glycolyl-CoA min⁻¹ mg⁻¹. It is preferred that the herein described GCCs have a specific activity of about 2602±430 nmol glycolyl-CoA min⁻¹ mg⁻¹. Accordingly, the present invention provides a GCC, wherein the specific activity of the GCC is about 2602±430 nmol glycolyl-CoA min⁻¹ mg⁻¹. In a preferred aspect, the present invention relates to a GCC comprising SEQ ID NO: 3, wherein the specific activity of the GCC is about 2602±430 nmol glycolyl-CoA min⁻¹ mg⁻¹. In another preferred aspect, the present invention relates to a GCC comprising SEQ ID NO: 3, wherein the specific activity of the GCC is about 2602±430 nmol glycolyl-CoA min⁻¹ mg⁻¹ and wherein the GCC has a catalytic rate of carboxylation of glycolyl-CoA (conversion of glycolyl-CoA into tartronyl-CoA) of higher than about 5.6±0.3 s⁻¹. In another preferred aspect, the present invention relates to a GCC comprising SEQ ID NO: 3, wherein the specific activity of the GCC is higher than about 937±40 nmol glycolyl-CoA min⁻¹ mg⁻¹ and wherein the GCC has a catalytic rate of carboxylation of glycolyl-CoA (conversion of glycolyl-CoA into tartronyl-CoA) of higher than about 5.6±0.3 s⁻¹.

The skilled person is well aware how carboxylation activity can be determined, e.g. by the assays described in the appended examples.

Carboxylation activity may be determined via lysate-based measurements in plate readers or via spectrophotometric measurements with purified enzymes (Scheffen, loc. cit.).

Lysate-based measurements may be performed as described in the following. GCC-encoding constructs or random-mutagenesis libraries of GCC may be transformed into E. coli BL21_birA (see above) and eight colonies per construct may be picked into 96-deep-well plates (PlateOne) with lysogeny broth (Miller) containing 100 μg/mL ampicillin and 50 μg/mL streptomycin. The plates may be incubated over night at 37° C. with subsequent transfer into fresh 96-deep-well plates with lysogeny broth (Miller), 100 μg/mL ampicillin, 50 μg/mL spectinomycin, and 2 μg/mL biotin to an OD₆₀₀ of 0.1. Protein expression may be induced with 0.25 mM isopropyl β-d-1-thiogalactopyranoside at an OD₆₀₀ of 0.4-0.6 and the cells may be incubated over night at 25° C. The cells may be lysed using CelLytic B (Sigma-Aldrich) and stored in 20% glycerol at −80° C. The enzyme activity may be measured in a plate reader by the coupled enzyme assay with purified Malonyl-CoA Reductase from Chloroflexus aurantiacus (also referred to as CaMCR) as described earlier (Scheffen, loc. cit.). Small-volume 384-well plates (Greiner Bio-One) with 2 μL cell extract, 100 mM 3-(N-morpholino)propanesulfonic acid (MOPS) pH 7.8, 1 mM ATP, 50 mM KHCO₃, 500 μg/mL CaMCR, 1 mM NADPH, 10 mM MgCl₂, and 1 mM glycolyl-CoA in a reaction volume of 10 μL may be used. The absorbance of NADPH may be measured at 340 nm and 37° C. for 5 h with intervals of 47 s in a plate reader (Tecan Infinite M Plex).

Spectrophotometric measurements of the carboxylation activity with purified enzymes may be performed as described in the following. The production and purification of enzymes and the synthesis of CoA esters is described elsewhere herein. To measure the carboxylation rate of GCC, a coupled enzyme assay with CaMCR may be performed. 100 mM MOPS pH 7.8, 50 mM KHCO₃, 2 mM ATP, 0.3 mM NADPH, 5 mM MgCl₂, 1.8 mg/mL CaMCR from Chloroflexus aurantiacus, and 0.01-1 mg/mL GCC may be mixed in a cuvette and incubated for 2 min at 37° C. The reaction may be started with 0.5 mM glycolyl-CoA and absorption may be measured over time at λ=340 nm.

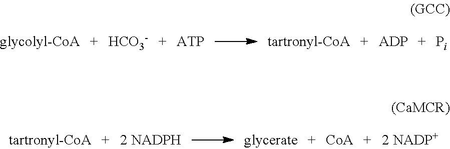

It is evident for the skilled person that the invention also encompasses GCCs with certain sequence identities to the herein described specific sequences.

For example, the invention provides a GCC, wherein said GCC is characterized in that: it comprises an amino acid sequence having at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or at least 99.8% sequence identity to SEQ ID NO: 1, and
it has one or more amino acid substitutions, deletions or insertions at a position selected from the group consisting of positions 20 and 100 in the amino acid sequence shown in SEQ ID NO: 1 or at a position corresponding to any of these positions,
preferably wherein the GCC has an improved activity over a reference GCC, preferably wherein the reference GCC has the amino acid sequence of SEQ ID NO: 1.

The skilled person is well aware how to determine percent identity between/among sequences using, for example, algorithms such as those based on CLUSTALW computer program (Thompson, Nucl. Acids Res. 2, 4673-4680, 1994), CLUSTAL Omega (Sievers, Curr. Protoc. Bioinformatics 48, 3.13.1-3.13.16, 2014) or FASTDB (Brutlag, Comp. App. Biosci. 6, 237-245, 1990). Also available to the skilled person are the BLAST, which stands for Basic Local Alignment Search Tool, and BLAST 2.0 algorithms (Altschul, Nucl. Acids Res. 25:3389-3402, 1997; Altschul, J. Mol. Biol. 215, 403-410, 1990) and related tools.

It is envisaged that a BLAST alignment of a sequence of SEQ ID NO: 1 with a sequence of SEQ ID NO: 1 with one amino acid substitution may result in a sequence identity of 99.8%.

The invention provides a GCC, wherein said GCC is characterized in that:
it comprises an amino acid sequence having at least 60% sequence identity to SEQ ID NO: 1, and
has an arginine at position 20 in the amino acid sequence shown in SEQ ID NO: 1 or at a position corresponding to this position,
preferably wherein the GCC has an improved activity over a reference GCC, preferably wherein the reference GCC has the amino acid sequence of SEQ ID NO: 1.

In a preferred aspect, the invention provides a GCC, wherein said GCC is characterized in that: it comprises the amino acid sequence of SEQ ID NO: 3, and
preferably wherein the GCC has an improved activity over a reference GCC, preferably wherein the reference GCC comprises the amino acid sequence of SEQ ID NO: 1.

In another preferred aspect, the invention provides a GCC, wherein said GCC is characterized in that:
it comprises a α-subunit comprising or consisting of the amino acid sequence of SEQ ID NO: 13 and/or it comprises a β-subunit comprising or consisting of SEQ ID NO: 3,
preferably wherein the GCC has an improved activity over a reference GCC, preferably wherein the reference GCC has the amino acid sequence of SEQ ID NO: 1.

The invention provides a GCC, wherein said GCC is characterized in that:
it comprises an amino acid sequence having at least 60% sequence identity to SEQ ID NO: 1, and
has an asparagine at position 100 in the amino acid sequence shown in SEQ ID NO: 1 or at a position corresponding to this position,
preferably wherein the GCC has an improved activity over a reference GCC, preferably wherein the reference GCC has the amino acid sequence of SEQ ID NO: 1.

In preferred aspect, the invention provides a GCC, wherein said GCC is characterized in that: it comprises the amino acid sequence of SEQ ID NO: 7, and
preferably wherein the GCC has an improved activity over a reference GCC, preferably wherein the reference GCC has the amino acid sequence of SEQ ID NO: 1.

In another preferred aspect, the invention provides a GCC, wherein said GCC is characterized in that:
it comprises an α-subunit comprising or consisting of the amino acid sequence of SEQ ID NO: 13 and/or it comprises a β-subunit comprising or consisting of SEQ ID NO: 7,
preferably wherein the GCC has an improved activity over a reference GCC, preferably wherein the reference GCC has the amino acid sequence of SEQ ID NO: 1.

As explained in detail herein, the present inventors constructed GCC M5 from the naturally occurring PCC by introducing five mutations into PCC (100S, Y143H, D407I, I450V and W502R). Accordingly, it is preferred that the herein described GCCs may have at one or more of said five positions (e.g. at one, two, three, four or five) the amino acids as in GCC M5 (shown in SEQ ID NO:1). It is envisaged that the herein described GCCs have one or more of (e.g. one, two, three, four or five of) a serine at position 100 (if the GCC variant has further mutations at positions other than those mutated in GCC M5 versus PCC), a histidine at position 143, an isoleucine at position 407, a valine at position 450 and/or an arginine at position 502 in the amino acid sequence shown in SEQ ID NO: 1 or at a position corresponding to any of these positions. It is evident for the skilled person that when position 100 is substituted to e.g. asparagine the position 100 has a different amino acid than serine, e.g. asparagine. For example, when a GCC has an amino acid substitution at position 100 (preferably an asparagine), it is envisaged that the GCC has a histidine at position 143, an isoleucine at position 407, a valine at position 450 and/or an arginine at position 502 in the amino acid sequence shown in SEQ ID NO: 1 or at a position corresponding to any of these positions. However, it is envisaged that a GCC in which position 20 in SEQ ID NO: 1 or a position corresponding to this position is substituted said GCC has a serine at position 100, a histidine at position 143, an isoleucine at position 407, a valine at position 450 and/or an arginine at position 502 in the amino acid sequence shown in SEQ ID NO: 1 or at a position corresponding to any of these positions.

In accordance with the above, a GCC disclosed herein can have—apart from the mutations described herein—a certain variation, for example the further modifications described further below. This is meant and implied by the language “at least 60% identity” and the like as used herein.

It is understood that a GCC disclosed herein which is characterized in that it comprises an amino acid sequence having at least 60% sequence identity to SEQ ID NO: 1, and which is disclosed to have one or more of the disclosed mutations/substitutions, e.g. one or more of an arginine at position 20, a serine at position 100, a histidine at position 143, an isoleucine at position 407, a valine at position 450 and/or an arginine at position 502 in the amino acid sequence shown in SEQ ID NO: 1 or at a position corresponding to any of these positions means that the GCC has the mutations/substitutions as constituent factor, i.e. they need to be present in the amino acid sequence of the GCC. However, due to the at least 60% sequence identity to SEQ ID NO: 1 the GCC can have further mutations/substitutions at other positions within the threshold of at least 60% sequence identity to SEQ ID NO: 1. For example, in addition to one or more mentioned mutations, the amino acid sequence of the GCC can have further modifications, e.g. substitutions, additions and/or deletions of one or more amino acid(s) (preferably substitutions), preferably of 1, 2, 3, 4, 5, 6, 7, 8, 9 or 10 or more amino acid(s), like 11, 12, 13, 14, 15, 16, 17, 18, 19 or 20 amino acids. For example, the amino acid sequence of the GCC can have further such modifications at positions corresponding to positions 1 to 19, 20, 21-99, 100, 101-142, 143, 144-406, 407, 408-449, 450, 451-501, 502, 503-510. For example, if the GCC has (only) a mutation as disclosed above at position 20, it can have one or more further mutations at any other position, e.g. at one or more positions 1-19 and/or 21-510. As another example, if a GCC has one or more of the disclosed mutations/substitutions, e.g. one or more of an arginine at position 20, a histidine at position 143, an isoleucine at position 407, a valine at position 450 and/or an arginine at position 502 in the amino acid sequence shown in SEQ ID NO: 1 or at a position corresponding to any of these positions the GCC can have one or more mutations/substitutions e.g. at positions corresponding to positions 1 to 19, 21-142, 144-406, 408-449, 451-501, and/or 503-510.

Particularly such further modifications (mutations/substitutions) are envisaged that do not affect the activity of the GCC as defined herein or that do not substantially affect the activity of the GCC as defined herein. “Not substantially affect” may mean in this context a decrease of the activity of the GCC of up to 10% compared to the GCC not carrying/having such modifications. Specifically, a GCC with such further modifications (mutations/substitutions) still shows improved activity over a reference GCC, preferably wherein the reference GCC comprises the amino acid sequence of SEQ ID NO:1.

It is understood that the GCC, provided and to be used herein, comprises the mutation(s) as defined and explained herein, specifically one or more amino acid substitutions at positions 20 or 100 in the amino acid sequence shown in SEQ ID NO: 1 or at a position corresponding to any of these positions. Apart from these mutations, it is envisaged herein that the sequence of the mutant GCC at all other positions can comprise or consist of an amino acid sequence identical to that of its counterpart (GCC-M5, SEQ ID NO. 1). For example, if the GCC comprises the mutation(s) as defined and explained herein, it can comprise or consist of an amino acid sequence outside of these mutations that is identical to that of GCC-M5, SEQ ID NO. 1.

Preferably, any such further modifications (e.g. deletions, insertions, additions and/or substitutions (in this context particularly substitutions) are conservative, i.e. amino acids are substituted by amino acids having the same or similar characteristics. For example, a hydrophobic amino acid will preferably be substituted by another hydrophobic amino acid and so on.

The invention provides a GCC, wherein said GCC is characterized in that:
it comprises an amino acid sequence having at least 60% sequence identity to SEQ ID NO: 1, and
it has one or more amino acid substitutions, deletions, or insertions at a position selected from the group consisting of positions 20 and 100 in the amino acid sequence shown in SEQ ID NO: 1 or at a position corresponding to any of these positions,
wherein the amino acid at position 502 is an arginine,
preferably wherein the GCC has an improved activity over a reference GCC, preferably wherein the reference GCC has the amino acid sequence of SEQ ID NO: 1.

The invention also provides a GCC, wherein said GCC is characterized in that:
it comprises an amino acid sequence having at least 60% sequence identity to SEQ ID NO: 1, and
it has one or more amino acid substitutions, deletions, or insertions at a position selected from the group consisting of positions 20 and 100 in the amino acid sequence shown in SEQ ID NO: 1 or at a position corresponding to any of these positions,
wherein the amino acid at position 450 is a valine,
preferably wherein the GCC has an improved activity over a reference GCC, preferably wherein the reference GCC has the amino acid sequence of SEQ ID NO: 1.

The invention also provides a GCC, wherein said GCC is characterized in that:
it comprises an amino acid sequence having at least 60% sequence identity to SEQ ID NO: 1, and
it has one or more amino acid substitutions, deletions, or insertions at a position selected from the group consisting of positions 20 and 100 in the amino acid sequence shown in SEQ ID NO: 1 or at a position corresponding to any of these positions,
wherein the amino acid at position 407 is an isoleucine,
preferably wherein the GCC has an improved activity over a reference GCC, preferably wherein the reference GCC has the amino acid sequence of SEQ ID NO: 1.

The invention further provides a GCC, wherein said GCC is characterized in that:
it comprises an amino acid sequence having at least 60% sequence identity to SEQ ID NO: 1, and
it has one or more amino acid substitutions, deletions, or insertions at a position selected from the group consisting of positions 20 and 100 in the amino acid sequence shown in SEQ ID NO: 1 or at a position corresponding to any of these positions,
wherein the amino acid at position 143 is a histidine,
preferably wherein the GCC has an improved activity over a reference GCC, preferably wherein the reference GCC has the amino acid sequence of SEQ ID NO: 1.

The invention further provides a GCC, wherein said GCC is characterized in that:
it comprises an amino acid sequence having at least 60% sequence identity to SEQ ID NO: 1, and
it has one or more amino acid substitutions, deletions, or insertions at a position selected from the group consisting of positions 20 and 100 in the amino acid sequence shown in SEQ ID NO: 1 or at a position corresponding to any of these positions,
wherein the amino acid at position 143 is a histidine, the amino acid at position 407 is an isoleucine, the amino acid at position 450 is a valine and/or the amino acid at position 502 is an arginine,
preferably wherein the GCC has an improved activity over a reference GCC, preferably wherein the reference GCC has the amino acid sequence of SEQ ID NO: 1.

The invention further provides a GCC, wherein said GCC is characterized in that:
it comprises an amino acid sequence having at least 60% sequence identity to SEQ ID NO: 1, and
it has an amino acid substitution, deletion, or insertion at position 20 in the amino acid sequence shown in SEQ ID NO: 1 or at a position corresponding to this position,
wherein the amino acid at position 100 is a serine, the amino acid at position 143 is a histidine, the amino acid at position 407 is an isoleucine, the amino acid at position 450 is a valine and/or the amino acid at position 502 is an arginine,
preferably wherein the GCC has an improved activity over a reference GCC, preferably wherein the reference GCC has the amino acid sequence of SEQ ID NO: 1.

The invention further provides a GCC, wherein said GCC is characterized in that:
it comprises an amino acid sequence having at least 60% sequence identity to SEQ ID NO: 1, and
it has an arginine at position 20 in the amino acid sequence shown in SEQ ID NO: 1 or at a position corresponding to this position,
wherein the amino acid at position 100 is a serine, the amino acid at position 143 is a histidine, the amino acid at position 407 is an isoleucine, the amino acid at position 450 is a valine and/or the amino acid at position 502 is an arginine,
preferably wherein the GCC has an improved activity over a reference GCC, preferably wherein the reference GCC has the amino acid sequence of SEQ ID NO: 1.

The invention further provides a GCC, wherein said GCC is characterized in that:
it comprises an amino acid sequence having at least 60% sequence identity to SEQ ID NO: 1, and
it has an arginine at position 20 in the amino acid sequence shown in SEQ ID NO: 1 or at a position corresponding to any of these positions,
wherein the amino acid at position 100 is an alanine, arginine, aspartate, cysteine, glutamine, glutamate, glycine, histidine, isoleucine, leucin, lysine, methionine, phenylalanine, proline, serine, threonine, tryptophan, tyrosine or valine, the amino acid at position 143 is a histidine, the amino acid at position 407 is an isoleucine, the amino acid at position 450 is a valine and/or the amino acid at position 502 is an arginine,
preferably wherein the GCC has an improved activity over a reference GCC, preferably wherein the reference GCC has the amino acid sequence of SEQ ID NO: 1.

The invention further provides a GCC, wherein said GCC is characterized in that:
it comprises an amino acid sequence having at least 60% sequence identity to SEQ ID NO: 1, and
it has an amino acid substitution, deletion, or insertion at position 100 in the amino acid sequence shown in SEQ ID NO: 1 or at a position corresponding to this position,
wherein, the amino acid at position 143 is a histidine, the amino acid at position 407 is an isoleucine, the amino acid at position 450 is a valine and/or the amino acid at position 502 is an arginine,
preferably wherein the GCC has an improved activity over a reference GCC, preferably wherein the reference GCC has the amino acid sequence of SEQ ID NO: 1.

The invention further provides a GCC, wherein said GCC is characterized in that:
it comprises an amino acid sequence having at least 60% sequence identity to SEQ ID NO: 1, and
it has an asparagine at position 100 in the amino acid sequence shown in SEQ ID NO: 1 or at a position corresponding to this position,
wherein, the amino acid at position 143 is a histidine, the amino acid at position 407 is an isoleucine, the amino acid at position 450 is a valine and/or the amino acid at position 502 is an arginine,
preferably wherein the GCC has an improved activity over a reference GCC, preferably wherein the reference GCC has the amino acid sequence of SEQ ID NO: 1.

The invention further provides a GCC, wherein said GCC is characterized in that:
it comprises an amino acid sequence having at least 60% sequence identity to SEQ ID NO: 1, and
it has an asparagine at position 100 in the amino acid sequence shown in SEQ ID NO: 1 or at a position corresponding to any of these positions,
wherein the amino acid at position 20 is an alanine, asparagine, aspartate, cysteine, glutamine, glutamate, glycine, histidine, isoleucine, leucin, lysine, methionine, phenylalanine, proline, serine, threonine, tryptophan, tyrosine or valine, the amino acid at position 143 is a histidine, the amino acid at position 407 is an isoleucine, the amino acid at position 450 is a valine and/or the amino acid at position 502 is an arginine,
preferably wherein the GCC has an improved activity over a reference GCC, preferably wherein the reference GCC has the amino acid sequence of SEQ ID NO: 1.

As evident from the aspects above, the described sequence identity to SEQ ID NO: 1 may not refer to all positions of SEQ ID NO: 1. In other words, at certain positions the amino acids should not be substituted although the resulting GCC would have e.g. at least 60% sequence identity to SEQ ID NO: 1. In particular the amino acid substitutions that lead from PCC to GCC M5 may not be further substituted.

As already explained in detail the present inventors found that amino acid substitutions at certain positions improve the activity of GCC M5. Thus, the invention provides a GCC with specific amino acid substitutions for the amino acids glycine and serine in positions 20 and 100, respectively, with respect to the reference amino acid sequence of SEQ ID NO: 1.

Accordingly, the invention provides a GCC wherein said GCC is characterized in that:
it comprises an amino acid sequence having at least 60% sequence identity to SEQ ID NO: 1, and
(1) the amino acid at position 20 in the amino acid sequence shown in SEQ ID NO: 1 or at a position corresponding to this position, is substituted with alanine, arginine, asparagine, aspartate, cysteine, glutamine, glutamate, histidine, isoleucine, leucine, lysine, methionine, phenylalanine, proline, serine, threonine, tryptophan, tyrosine or valine,
preferably with arginine, histidine, lysine or tyrosine,
more preferably with arginine,
preferably wherein the amino acid that is substituted is glycine; and/or
(2) the amino acid at position 100 in the amino acid sequence shown in SEQ ID NO: 1 or at a position corresponding to this position, is substituted with alanine, arginine, asparagine, aspartate, cysteine, glutamine, glutamate, glycine, histidine, isoleucine, leucin, lysine, methionine, phenylalanine, proline, threonine, tryptophan, tyrosine or valine,
preferably with asparagine,
preferably wherein the amino acid that is substituted is serine.

The invention also provides a GCC wherein said GCC is characterized in that:
it comprises an amino acid sequence having at least 60% sequence identity to SEQ ID NO: 1, and
(1) the amino acid at position 20 in the amino acid sequence shown in SEQ ID NO: 1 or at a position corresponding to this position, is substituted with alanine, arginine, asparagine, aspartate, cysteine, glutamine, glutamate, histidine, isoleucine, leucine, lysine, methionine, phenylalanine, proline, serine, threonine, tryptophan, tyrosine or valine,
preferably with arginine, histidine, lysine or tyrosine,
more preferably with arginine,
preferably wherein the amino acid that is substituted is glycine; and/or
(2) the amino acid at position 100 in the amino acid sequence shown in SEQ ID NO: 1 or at a position corresponding to this position, is substituted with alanine, arginine, asparagine, aspartate, cysteine, glutamine, glutamate, glycine, histidine, isoleucine, leucin, lysine, methionine, phenylalanine, proline, threonine, tryptophan, tyrosine or valine,
preferably with asparagine,
preferably wherein the amino acid that is substituted is serine;
preferably wherein the GCC has an improved activity over a reference GCC, preferably wherein the reference GCC has the amino acid sequence of SEQ ID NO:1.

The invention also provides a GCC wherein said GCC is characterized in that:
it comprises an amino acid sequence having at least 60% sequence identity to SEQ ID NO: 1, and
(1) the amino acid at position 20 in the amino acid sequence shown in SEQ ID NO: 1 or at a position corresponding to this position, is substituted with arginine,
preferably wherein the amino acid that is substituted is glycine; and/or
(2) the amino acid at position 100 in the amino acid sequence shown in SEQ ID NO: 1 or at a position corresponding to this position, is substituted with asparagine,
preferably wherein the amino acid that is substituted is serine.

The invention also provides a GCC wherein said GCC is characterized in that:
it comprises an amino acid sequence having at least 60% sequence identity to SEQ ID NO: 1, and
(1) the amino acid at position 20 in the amino acid sequence shown in SEQ ID NO: 1 or at a position corresponding to this position, is substituted with arginine,
preferably wherein the amino acid that is substituted is glycine; and/or
(2) the amino acid at position 100 in the amino acid sequence shown in SEQ ID NO: 1 or at a position corresponding to this position, is substituted with asparagine,
preferably wherein the amino acid that is substituted is serine;
preferably wherein the GCC has an improved activity over a reference GCC, preferably wherein the reference GCC has the amino acid sequence of SEQ ID NO:1.

The invention also relates to nucleic acid molecules encoding the herein described GCCs. In context of the present invention the nucleic acid molecule may be a DNA molecule or an RNA molecule.

In a preferred aspect, the present invention relates to a nucleic acid molecule as shown in SEQ ID NO: 16. In a preferred aspect the α-subunit of the GCC of the present invention is encoded by a nucleic acid molecule as shown in SEQ ID NO: 26 and the β-subunit is encoded a nucleic acid molecule as shown in SEQ ID NO: 16.

In another preferred aspect, the present invention relates to a nucleic acid molecule as shown in SEQ ID NO: 20. In a preferred aspect the α-subunit of the GCC of the present invention is encoded by a nucleic acid molecule as shown in SEQ ID NO: 26 and the β-subunit is encoded a nucleic acid molecule as shown in SEQ ID NO: 20.

The present invention further relates to a vector comprising the herein described nucleic acid molecules. Vectors that can be used in accordance with the present invention are known in the art. The vectors can further comprise expression control sequences operably linked to the nucleic acid molecules of the present invention contained in the vectors. These expression control sequences may be suited to ensure transcription and synthesis of a translatable RNA in bacteria or fungi. Expression control sequences can for instance be promoters. Promoters for use in connection with the nucleic acid molecules of the present invention may be homologous or heterologous with regard to its origin and/or with regard to the gene to be expressed. Suitable promoters are for instance promoters which lend themselves to constitutive expression. However, promoters which are only activated at a point in time determined by external influences can also be used. Artificial and/or chemically inducible promoters may be used in this context.

Preferably, the vector of the present invention is an expression vector. Expression vectors have been widely described in the literature. As a rule, they contain not only a selection marker gene and a replication-origin ensuring replication in the host selected, but also a bacterial or viral promoter, and in most cases a termination signal for transcription. Between the promoter and the termination signal there is in general at least one restriction site or a polylinker which enables the insertion of a coding DNA sequence. The DNA sequence naturally controlling the transcription of the corresponding gene can be used as the promoter sequence, if it is active in the selected host organism. However, this sequence can also be exchanged for other promoter sequences. It is possible to use promoters ensuring constitutive expression of the gene and inducible promoters which permit a deliberate control of the expression of the gene. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of a downstream sequence are for instance the T7 promoter (Studier, Methods in Enzymology 185, 60-89, 1990), lacUV5, trp, trp-lacUV5 (DeBoer, Promoters, Structure and Function, Praeger, 462-481, 1982; DeBoer, Proc. Natl. Acad. Sci. USA 80, 21-25, 1983), Ip1, or rac (Boros, Gene 42, 97-100, 1986). Inducible promoters are preferably used for the expression of downstream sequences. These promoters often lead to higher polypeptide yields than do constitutive promoters. In order to obtain an optimum amount of polypeptide, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (=isopropyl-β-D-thiogalactopyranoside) (DeBoer, loc. cit.). Termination signals for transcription are also described in the literature.

The present invention further provides an organelle comprising the nucleic acid molecule or the vector as described herein.

Thus, the invention provides an organelle comprising a nucleic acid molecule encoding the GCC of the invention or a vector comprising a nucleic acid molecule encoding the GCC of the invention. Thus, the invention provides an organelle producing the GCC of the present invention. An organelle is a specialized subunit within a cell, usually an eukaryotic cell, that performs a specific function. Examples of organelles are the mitochondrion, nucleus, Golgi apparatus, endoplasmic reticulum and plastids. Preferably, an organelle in the context of the invention is a plastid. A plastid can be, among others, a chloroplast, cyanoplast, rhodoplast, or a phaeoplast, preferably a chloroplast.

The invention also provides a host cell comprising the herein described nucleic acid molecule, the herein described vector or the herein described organelle. The skilled person is readily capable of choosing suitable host cells, e.g. for protein expression according to various factors such as the desired protein, the level of expression required, the downstream processing requirements, and the cost of the process. Commonly used host cells for protein expression include bacteria (such as E. coli), yeast (such as Saccharomyces cerevisiae), insect cells (such as Sf9 and Sf21), and mammalian cells (such as CHO and HEK293). Each host cell has its advantages and limitations. For example, bacterial systems are relatively easy to grow and produce high yields of protein, but they may not be suitable for expressing complex eukaryotic proteins with post-translational modifications. Yeast systems are also easy to grow and have the ability to perform some post-translational modifications, but they may not be suitable for certain types of proteins. Insect cells have the ability to perform some post-translational modifications. They are suitable for expressing large, complex proteins but are more expensive to culture than bacterial and yeast systems. Mammalian cells are often preferred for producing biologically active and properly folded proteins with complex post-translational modifications. Still, they are more difficult and expensive to culture than bacterial and yeast systems. Ultimately, the choice of the host cell for protein expression will depend on the specific requirements of the protein and the downstream application.

If the host cell is used as a chassis for the cloning and construction of CO₂ fixation modules or for the expression of any proteins related to CO₂ fixation or photosynthesis that do not rely on posttranslational modification, E. coli is the preferred organism because of its genetic accessibility, easy cultivability, and fast growth. The cyanobacteria strains Synechococcus elongatus and Synechocystis sp. PCC 6803 are the preferred cells to express proteins in the background of photosynthetic metabolism because these organisms are genetically accessible, easy to cultivate, grow fast and are less complex than eukaryotic phototrophic organisms. Single celled algae like Chlamydomonas reinhardtii are preferred to express proteins in the background of photosynthetic metabolism in eukaryotic cells or in chloroplasts because they have a comparable metabolism and physiology to plants but grow faster and are easier to cultivate. The preferred organism to express proteins in the background of photosynthesis in plants is Arabidopsis thaliana because of its fast growth compared to other plants or any crop plant because of its industrial value.

The invention also provides a tissue comprising the herein-described host cell. Accordingly, the invention provides a tissue comprising a host cell comprising the herein-described nucleic acid molecule, the herein-described vector or the herein-described organelle. Tissue may be defined as an association of similar or diverse host cells described herein that perform a specific function. The host cells described herein may be seeded onto a scaffold, which serves as a three-dimensional support structure for the cells to grow and e.g. produce the described GCC.

The invention further provides an organism comprising the herein described nucleic acid molecule, vector or organelle. Furthermore, the present invention relates to an organism or another related biological system comprising or encoding the described GCCs. Preferably, the organism in context of the invention is a plant, an algae, or a microorganism.

The algae are not particularly limited, but are eukaryotic unicellular organisms (diatoms, yellow-green algae, dinoflagellate, etc.) or multicellular organisms such as sea algae (red algae, brown algae, green algae), etc. They are found in a variety of environments, including oceans, freshwater bodies, soil, and even on other organisms. The skilled person is readily capable to select suitable algae for the desired application.

Algae comprising the herein described nucleic acid molecule, vector or organelle may be used for biofuel production, food production, bioplastic production or carbon sequestration in general. One approach to algae-based carbon sequestration is to grow algae in large-scale, closed systems, such as photobioreactors or ponds. In these systems, algae are grown under controlled conditions and are provided with the nutrients and light necessary for photosynthesis. As the algae grow, they absorb CO₂ from the air and convert it into biomass. Once the algae have reached maturity, they can be harvested and processed to produce biofuels, food, or other products, while the remaining biomass can be used as a soil amendment or stored for long-term carbon sequestration. Another approach is to use algae to capture and recycle CO₂ emissions from industrial sources, such as power plants or factories. In this process, flue gas from industrial emissions is directed to algae ponds or bioreactors, where the algae absorb the CO₂ and convert it into biomass. This approach has the potential to reduce greenhouse gas emissions from industrial sources and produce renewable, carbon-neutral or carbon-negative products.

The plant which may be used in context of the present invention is not limited and may belong to dicotyledonous plants or monocotyledonous plants. Non-limiting examples of plants that may be used in context of the invention are provided below.
Brassicaceae: Arabidopsis thaliana (Arabidopsis thaliana), Brassica rapa (Brassica rapa, Brassica napus), Cabbage (Brassica oleracea var. Capitata), Chinese Cabbage (Brassica rapa var. Pekinensis), rosewood (Brassica rapa var. hakabura), Mizuna (Brassica rapa var. lanciniifolia), Komatsuna (Brassica rapa var. perviridis), Japanese radish (Raphanus sativus), wasabi (Wasabia japonica) and the like. Solanaceae: tobacco (Nicotiana tabacum), eggplant (Solanum melongena), potato (Solanum tuberosum), tomato (Lycopersicon lycopersicum), pepper (Capsicum annuum), petunia, etc. Legumes: soybean (Glycine max), pea (Pisum sativum), broad bean (Vicia faba), wisteria (Wisteria floribunda), groundnut (Arachis hypogaea), lotus root (Lotus corniculatus var. Japonicus), kidney bean (Phaseolus vulgaris), azuki bean (Vigna angularis), acacia (Acacia) etc. Asteraceae: Chrysanthemum morifolium, sunflower (Helianthus annuus), etc. Palm family: oil palm (Elaeis guineensis, Elaeis oleifera), coconut (Cocos nucifera), date palm (Phoenix dactylifera), wax palm (Copernicia) and the like. Urushi family: Lotus tree (Rhus succedanea), cashew (Anacardium occidentale), Urushi (Toxicodendron vernicifluum), mango (Mangifera indica), pistachio (Pistacia vera) and the like. Cucurbitaceae: squash (Cucurbita maxima, Cucurbita moschata, Cucurbita pepo), cucumber (Cucumis sativus), currant (Trichosanthes cucumeroides), gourd (Lagenaria siceraria var. Gourda) and the like. Rosaceae: Almond (Amygdalus communis), rose (Rosa), strawberry (Fragaria), cherry (Prunus), apple (Malus pumila var. Domestica) and the like. Willow family: Poplar (Populus trichocarpa, Populus nigra, Populus tremula) and the like. Grasses: Brachypodium, Maize (Zea mays), rice (Oryza sativa), barley (Hordeum vulgare), wheat (Triticum aestivum), bamboo (Phyllostachys), sugarcane (Saccharum officinarum), napiergrass (Pennisetum putureum), Miscanthus (Miscanthus virgatum), Sorghum (Sorghum) switchgrass (Panicum) and the like. Lilium family: tulip (Tulipa), lily (Lilium) and the like. Myrtaceae family: Eucalyptus (Eucalyptus camaldulensis, Eucalyptus grandis) and the like.

A plant in context of the invention may also be a moss. Mosses are also sometimes referred to as bryophytes that contains three groups of non-vascular land plants (liverworts, hornworts, and mosses). A plant in context of the invention may also be a fern.

As mentioned above, the invention provides a microorganism comprising the herein-described nucleic acid molecule, vector, or organelle. A microorganism as used herein is not limited and refers to an organism of microscopic size, which may exist in its single-celled form or as a colony of cells. Microorganisms include, inter alia, bacteria, fungi, archaea and protists.

Non-limiting examples of microorganisms include Aspergillus such as Aspergillus aculeatus and Aspergillus oryzae. Also various known yeasts are envisaged e.g. of the genus Saccharomyces such as Saccharomyces cerevisiae, yeasts of the genus Schizosaccharomyces such as Schizosaccharomyces pombe, yeasts of the genus Candida, such as Candida shehatae, yeasts of the genus Pichia such as Pichia stipitis, yeasts of the genus Hansenula, yeasts of the genus Klocekera, yeasts of the genus Swaniomyces, yeasts of the genus Yarrowia, yeasts of the genus Trichosporon, yeasts of the genus Brettanomyces, or yeasts of the genus Pachysolen.

The microorganism in context of the invention may be a bacterium. Accordingly, the invention provides a bacterium comprising the herein described nucleic acid molecule or vector. The skilled person is well aware of suitable bacteria species for the desired application. Nonlimiting examples are species of the genus Escherichia, such as Escherichia coli, Bacillus, such as Bacillus subtilis, Streptomyces, and Pseudomonas, such as Pseudomonas putida.

Although evident for the skilled person it is pointed out that when an organelle, host cell, tissue or organism comprises a herein described nucleic acid it is envisaged that said organelle, host cell, tissue or organism expresses said nucleic acid, i.e. said organelle, host cell, tissue or organism produces the protein/polypeptide encoded by the nucleic acid. Accordingly, it is envisaged that an organelle, host cell, tissue or organism produces the herein described GCCs. In a preferred aspect of the invention an organelle, host cell, tissue or organism produces the protein of SEQ ID NO: 3 or 7. Accordingly, in a preferred aspect of the invention an organelle, host cell, tissue or organism comprises the nucleic acid of SEQ ID NO: 16 or 20. As mentioned before, a GCC comprises an α-subunit and a β-subunit. Accordingly, it is envisaged an organelle, host cell, tissue or organism produces an α-subunit and a β-subunit of a GCC. Accordingly, in a preferred aspect of the invention an organelle, host cell, tissue or organism comprises the nucleic acid of SEQ ID NO: 26 and the nucleic acid of SEQ ID NO: 16 or 20.

It is envisaged that an organelle, host cell, tissue or organism that produces the GCCs of the invention may be characterized through increased conversion rate of glycolyl-CoA into tartronyl-CoA as compared to an organelle, host cell, tissue or organism producing a GCC of the prior art.

Accordingly, the present invention provides an organelle, host cell, tissue or organism that produces a GCC of the invention, wherein said organelle, said host cell, said tissue or said organism has a higher conversion rate of glycolyl-CoA into tartronyl-CoA than the corresponding organelle, host cell, tissue or organism producing a reference GCC, preferably a reference GCC comprising SEQ ID NO: 1.

Accordingly, the present invention provides an organelle, host cell, tissue or organism comprising a GCC of the invention, wherein said organelle, said host cell, said tissue or said organism has a higher conversion rate of glycolyl-CoA into tartronyl-CoA than the corresponding organelle, host cell, tissue or organism comprising a reference GCC, preferably a reference GCC comprising SEQ ID NO: 1.

Higher conversion rates of glycolyl-CoA into tartronyl-CoA resulting from production of the described GCCs consequently may result in higher carboxylation rates and thereby in higher carbon yields which might have effects towards an improved growth rate of e.g. organelles, host cells, tissues or organisms. The growth rate may be directly linked to the accumulation of biomass and can be quantified, inter alia, as proliferation, expansion, or multiplication of e.g. organelles, host cells, tissues or organisms. Methods for quantification of carbon yields and improved growth/higher growth rate are known in the art.

Accordingly, the present invention provides an organelle, host cell, tissue or organism that produces a GCC of the invention, wherein said organelle, said host cell, said tissue or said organism has a higher growth rate and/or carbon yield than the corresponding organelle, host cell, tissue or organism producing a reference GCC, preferably a reference GCC comprising SEQ ID NO: 1.

Accordingly, the present invention provides an organelle, host cell, tissue or organism comprising a GCC of the invention, wherein said organelle, said host cell, said tissue or said organism has a higher growth rate and/or carbon yield than the corresponding organelle, host cell, tissue or organism comprising a reference GCC, preferably a reference GCC comprising SEQ ID NO: 1.

It is envisaged that the herein described GCCs, organelles, the host cells, the tissues or organisms are used for certain methods and processes.

Accordingly, the invention provides uses of the herein described GCCs, organelles, host cells, tissues or organisms. It is pointed out that all disclosed uses and methods for herein described GCCs, organelles, host cells, tissues or organisms are also disclosed herein for the described nucleic acids and vectors if they can be used in a context where the encoded polypeptides may be produced e.g. to catalyse carboxylation of glycolyl-CoA into tartronyl-CoA.

The described GCCs, organelles, host cells, tissues or organisms may be used in the fixation of CO₂ as they can catalyse the conversion of glycolyl-CoA into tartronyl-CoA through the carboxylation of glycolyl-CoA. Similarly, the described GCCs, organelles, host cells, tissues or organisms may be used for photosynthetic processes that also rely on the incorporation of CO₂ into organic molecules. CO₂-fixation and photosynthetic processes are defined by the incorporation of inorganic gaseous or aqueous CO₂ into organic molecules. Said processes may rely on the use of energy equivalents that were anabolized with the help of light energy. However, other means of provision of energy equivalents, such as ATP, may also be envisaged. Accordingly, the present invention provides a use of the herein described GCCs, organelles, the host cells, the tissues or organisms for CO₂-fixation and/or for a photosynthetic process.

The described GCCs, organelles, host cells, tissues or organisms may also be used for decomposition of certain substances that are for example harmful waste products. It is for example envisaged that the described GCCs, organelles, host cells, tissues or organisms may be used for the decomposition of synthetic material that pollutes the environment.

Accordingly, uses of the described GCCs, organelles, host cells, tissues or organisms may involve decomposition of synthetic material. Synthetic material refers non-limiting to a substance that is formulated or manufactured by a chemical process or by a process that chemically changes a substance extracted from naturally occurring plants, animals, or mineral sources.

Accordingly, the present invention provides a use of the described GCCs, organelles, host cells, tissues or organisms for decomposition of a synthetic material.

In context of the invention synthetic materials may for example be polyesters that are produced from monomers obtained from mineral oil. Accordingly, the described GCCs, organelles, host cells, tissues or organisms may be used in the decomposition of a polyester, such as a synthetic resin, or the like, in which monomer units are linked by ester groups. Polyesters and means for their decomposition are well known to the skilled person.

Accordingly, the present invention provides the use of the described GCCs, organelles, host cells, tissues or organisms for decomposition of a synthetic material, wherein the synthetic material is a polyester. In other words, the present invention provides the use of the described GCCs, organelles, host cells, tissues or organisms for decomposition of a polyester.

A widely used polyester based mainly on mineral oil sources that represents a major pollutant of the environment is polyethylene terephthalate (PET). PET is composed of ethylene terephthalate monomers that are interlinked via ester bonds to polymeric structures of variable length. Degradation of PET may constitute the hydrolytic cleavage of such ester bonds resulting in monomeric ethylene glycol and terephthalic acid. The person skilled in the art is well aware that ethylene glycol may be readily enzymatically modified to produce glycolyl-CoA which may in return be carboxylated by the described GCCs, organelles, host cells, tissues or organisms to produce tartronyl-CoA.

Accordingly, the present invention provides the use of the described GCCs, organelles, host cells, tissues or organisms for decomposition of a polyester, wherein the polyester is PET. In other words, the present invention provides the use of the described GCCs, organelles, host cells, tissues or organisms for decomposition of PET.

Ethylene glycol is also a relevant pollutant of the environment as it is widely used as deicing fluid for example for aircrafts. Besides that, one of the most abundant organic compounds in the ocean is glycolate as it is secreted by marine algae (Wright, Mar. Biol. 43, 257-263, 1977). The skilled person understands that ethylene glycol and or glycolate may be glycolyl-CoA precursors. In other words, given the appropriate catalytic conditions or presence of adequate enzymes ethylene glycol and or glycolate may be converted into glycolyl-CoA. Glycolyl-CoA in turn may be converted into tartronyl-CoA by the herein described GCCs, organelles, host cells, tissues or organisms. Accordingly, the herein described GCCs, organelles, host cells, tissues or organisms may be used for the decomposition of ethylene glycol and or glycolate. Suitable enzymes for the decomposition of ethylene glycol to glycolate are Gox0313 from Gluconobacter oxydans, FucO from E. coli or any homologous enzyme, suitable enzymes for the activation of glycolate to glycolyl-CoA are the recently developed glycolyl-CoA synthase GCS that was engineered from Erythrobacter sp. NAP1 or any homologous enzymes (Scheffen, loc. cit.). Usage of a CoA-transferase like AbfT from Clostridium aminobutyricum is also envisaged.

Accordingly, the present invention provides the use of the herein described GCCs, organelles, host cells, tissues or organisms for decomposition of ethylene glycol and/or glycolate. Although evident, it is pointed out that the skilled person can readily identify other substances or materials that may be degraded via glycolyl-CoA or a precursor of glycolyl-CoA. Accordingly, the skilled person can identify other substances or materials whose decomposition can be aided by the herein described GCCs, organelles, host cells, tissues or organisms.

The disclosures in context of the methods described herein are disclosed as corresponding uses mutatis mutandis. The disclosures in context of the uses described herein are disclosed as corresponding methods mutatis mutandis.

Accordingly, the present invention provides methods using the herein described GCCs, organelles, host cells, tissues or organisms for both catabolic and anabolic processes.

Accordingly, the present invention provides a method for the production of biomass using the herein described GCCs, organelles, host cells, tissues or organisms. The skilled person understands that a method for producing biomass can comprise the incorporation of inorganic carbon, such as CO₂, into organic molecules thereby resulting, inter alia, in the proliferation, expansion, or multiplication of the organelles, host cells, tissues or organisms.

Furthermore, the present invention provides a method using the herein described GCCs, organelles, host cells, tissues or organisms for the degradation of a synthetic material, preferably a polyester, more preferably PET.

Accordingly, the present invention provides a method for decomposition of a synthetic material using the herein described GCCs, organelles, host cells, tissues or organisms.

The present invention also provides a method for decomposition of polyester using the herein described GCCs, organelles, host cells, tissues or organisms.

The present invention also provides a method for decomposition of PET using the herein described GCCs, organelles, host cells, tissues or organisms.

The present invention also provides a method for decomposition of ethylene glycol and/or glycolate using the herein described GCCs, organelles, host cells, tissues or organisms.

It is also envisaged that the herein described nucleic acids, vectors, GCCs, organelles, host cells, tissues and/or organisms are comprised in compositions that may e.g. comprise additional reagents for a specific method or use.

The present invention also provides a composition comprising the herein described nucleic acids, vectors, GCCs, organelles, host cells, tissues and/or organisms.

The herein described uses and methods for the herein described nucleic acids, vectors, GCCs, organelles, host cells, tissues or organisms are disclosed for the corresponding compositions mutatis mutandis.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include”, “such as” and “and the like” are intended to convey inclusion without limitation, unless otherwise specifically indicated.

As used herein, the term “or” is generally employed in its usual sense including “and/or” unless the content clearly dictates otherwise. The term “and/or” means one or all of the listed elements or a combination of any two or more of the listed elements.

As used herein, the term “comprising” also specifically includes “consisting of” and “consisting essentially of” the recited elements, unless specifically indicated otherwise.

As used herein, the term “about” indicates and encompasses an indicated value and a range above and below that value. The term “about” may indicate the designated value±10%, ±5%, or ±1%. In certain embodiments, where applicable, the term “about” indicates the designated value(s)±one standard deviation of that value(s).

As used herein, the terms “comprising”, “including”, “having” or grammatical variants thereof are to be taken as specifying the stated features, integers, steps or components but do not preclude the addition of one or more additional features, integers, steps, components or groups thereof. The terms “comprising”/“including”/“having” encompass the terms “consisting of” and “consisting essentially of”. Thus, whenever the terms “comprising”/“including”/“having” are used herein, they can be replaced by “consisting essentially of” or, preferably, by “consisting of”.

The terms “comprising”/“including”/“having” mean that any further component (or likewise features, integers, steps and the like) can be present.

The term “consisting of” means that no further component (or likewise features, integers, steps and the like) can be present.

The term “consisting essentially of” or grammatical variants thereof when used herein are to be taken as specifying the stated features, integers, steps or components but do not preclude the addition of one or more additional features, integers, steps, components or groups thereof but only if the additional features, integers, steps, components or groups thereof do not materially alter the basic and novel characteristics of the claimed product, composition, use or method and the like.

The term “method” refers to manners, means, techniques and procedures for accomplishing a given task including, but not limited to, those manners, means, techniques and procedures either known to, or readily developed from known manners, means, techniques and procedures by practitioners of the chemical, biological and biophysical arts.

All sequences disclosed in this work are listed in the following table.

TABLE 1
Sequences

| Sequence ID | Sequence Name |
|---|---|
| SEQ ID NO: 1 | GCC M5 (M. extorquens) β-subunit L100S, Y143H, D407I, I450V, W502R |
| | mutations (PROTEIN) |
| SEQ ID NO: 2 | GCC M5_L8F (M. extorquens) β-subunit L8F, L100S, Y143H, D407I, I450V, |
| | W502R mutations (PROTEIN) |
| SEQ ID NO: 3 | GCC M5_G20R (M. extorquens) β-subunit G20R, L100S, Y143H, D407I, |
| | I450V, W502R mutations (PROTEIN) |
| SEQ ID NO: 4 | GCC M5_M53E (M. extorquens) β-subunit M53E, L100S, Y143H, D407I, |
| | I450V, W502R mutations (PROTEIN) |
| SEQ ID NO: 5 | GCC M5_M53Q (M. extorquens) β-subunit M53Q, L100S, Y143H, D407I, |
| | I450V, W502R mutations (PROTEIN) |
| SEQ ID NO: 6 | GCC M5_M64R (M. extorquens) β-subunit M64R, L100S, Y143H, D407I, |
| | I450V, W502R mutations (PROTEIN) |
| SEQ ID NO: 7 | GCC M5_L100N (M. extorquens) β-subunit L100N, Y143H, D407I, I450V, |
| | W502R mutations (PROTEIN) |
| SEQ ID NO: 8 | GCC M5_D407V (M. extorquens) β-subunit L100S, Y143H, D407V, I450V, |
| | W502R mutations (PROTEIN) |
| SEQ ID NO: 9 | GCC M5_T495N (M. extorquens) β-subunit L100S, Y143H, D407I, I450V, |
| | T495N, W502R mutations (PROTEIN) |
| SEQ ID NO: 10 | GCC M5_W502H (M. extorquens) β-subunit L100S, Y143H, D407I, I450V, |
| | W502H mutations (PROTEIN) |
| SEQ ID NO: 11 | GCC M5_L510F (M. extorquens) β-subunit L100S, Y143H, D407I, I450V, |
| | W502R, L510F mutations (PROTEIN) |
| SEQ ID NO: 12 | PCC WT (M. extorquens) β-subunit (PROTEIN) |
| SEQ ID NO: 13 | PCC WT (M. extorquens) α-subunit (PROTEIN) |
| SEQ ID NO: 14 | GCC M5 (M. extorquens) β-subunit L100S, Y143H, D407I, I450V, W502R |
| | mutations (DNA) |
| SEQ ID NO: 15 | GCC M5_L8F (M. extorquens) β-subunit L8F, L100S, Y143H, D407I, I450V, |
| | W502R mutations (DNA) |
| SEQ ID NO: 16 | GCC M5_G20R (M. extorquens) β-subunit G20R, L100S, Y143H, D407I, I450V, |
| | W502R mutations (DNA) |
| SEQ ID NO: 17 | GCC M5_M53E (M. extorquens) β-subunit M53E, L100S, Y143H, D407I, |
| | I450V, W502R mutations (DNA) |
| SEQ ID NO: 18 | GCC M5_M53Q (M. extorquens) β-subunit M53Q, L100S, Y143H, D407I, |
| | I450V, W502R mutations (DNA) |
| SEQ ID NO: 19 | GCC M5_M64R (M. extorquens) β-subunit M64R, L100S, Y143H, D407I, |
| | I450V, W502R mutations (DNA) |
| SEQ ID NO: 20 | GCC M5_L100N (M. extorquens) β-subunit L100N, Y143H, D407I, I450V, |
| | W502R mutations (DNA) |
| SEQ ID NO: 21 | GCC M5_D407V (M. extorquens) β-subunit L100S, Y143H, D407V, I450V, |
| | W502R mutations (DNA) |
| SEQ ID NO: 22 | GCC M5_T495N (M. extorquens) β-subunit L100S, Y143H, D407I, I450V, |
| | T495N, W502R mutations (DNA) |
| SEQ ID NO: 23 | GCC M5_W502H (M. extorquens) β-subunit L100S, Y143H, D407I, I450V, |
| | W502H mutations (DNA) |
| SEQ ID NO: 24 | GCC M5_L510F (M. extorquens) β-subunit L100S, Y143H, D407I, I450V, |
| | W502R, L510F mutations (DNA) |
| SEQ ID NO: 25 | PCC WT (M. extorquens) β-subunit (DNA) |
| SEQ ID NO: 26 | PCC WT (M. extorquens) α-subunit (DNA) |
| SEQ ID NO: 27 | Biotin ligase BirA (M. extorquens) (PROTEIN) |
| SEQ ID NO: 28 | Biotin ligase BirA (M. extorquens) (DNA) |
| SEQ ID NO: 29 | oDM0088 (DNA) |
| SEQ ID NO: 30 | oDM0115 (DNA) |
| SEQ ID NO: 31 | oDM0116 (DNA) |
| SEQ ID NO: 32 | oDM0117 (DNA) |
| SEQ ID NO: 33 | oDM0118 (DNA) |
| SEQ ID NO: 34 | oDM0119 (DNA) |
| SEQ ID NO: 35 | oDM0120 (DNA) |
| SEQ ID NO: 36 | oDM0121 (DNA) |
| SEQ ID NO: 37 | oDM0122 (DNA) |
| SEQ ID NO: 38 | oDM0123 (DNA) |
| SEQ ID NO: 39 | oDM0124 (DNA) |
| SEQ ID NO: 40 | oDM0143 (DNA) |
| SEQ ID NO: 41 | oDM0144 (DNA) |
| SEQ ID NO: 42 | oDM0145 (DNA) |
| SEQ ID NO: 43 | oDM0146 (DNA) |
| SEQ ID NO: 44 | oDM0164 (DNA) |
| SEQ ID NO: 45 | oDM0165 (DNA) |
| SEQ ID NO: 46 | oDM0166 (DNA) |
| SEQ ID NO: 47 | oDM0167 (DNA) |
| SEQ ID NO: 48 | oDM0168 (DNA) |
| SEQ ID NO: 49 | oDM0169 (DNA) |
| SEQ ID NO: 50 | PCC_seq1 (DNA) |
| SEQ ID NO: 51 | PCC_seq2_II (DNA) |
| SEQ ID NO: 52 | PCC_seq3_II (DNA) |
| SEQ ID NO: 53 | PCC_seq4 (DNA) |
| SEQ ID NO: 54 | PCC_seq5 (DNA) |
| SEQ ID NO: 55 | PCC_seq6 (DNA) |
| SEQ ID NO: 56 | PCC_seq7 (DNA) |
| SEQ ID NO: 57 | PccB_fw_P1 (DNA) |
| SEQ ID NO: 58 | PccB_rv_P1 (DNA) |

The present invention refers to the following nucleotide and amino acid sequences:

Amino acid sequences
Amino acid sequence of GCC M5 (M. extorquens) β-subunit L100S, Y143H,
D407I, I450V, W502R mutations
SEQ ID NO: 1
MKDILEKLEERRAQARLGGGEKRLEAQHKRGKLTARERIELLLDHGSFEEFDMFVQHRSTDFGMEKQKIP
GDGVVTGWGTVNGRTVFLFSKDFTVFGGSSSEAHAAKIVKVQDMALKMRAPIIGIFDAGGARIQEGVAA
LGGHGEVFRRNVAASGVIPQISVIMGPCAGGDVYSPAMTDFIFMVRDTSYMFVTGPDVVKTVTNEVVT
AEELGGAKVHTSKSSIADGSFENDVEAILQIRRLLDFLPANNIEGVPEIESFDDVNRLDKSLDTLIPDNPNKP
YDMGELIRRVVDEGDFFEIQAAYARNIITGFGRVEGRTVGFVANQPLVLAGVLDSDASRKAARFVRFCNA
FSIPIVTFVDVPGFLPGTAQEYGGLIKHGAKLLFAYSQATVPLVTIITRKAFGGAYIVMASKHVGADLNYAW
PTAQIAVMGAKGAVEIIFRAEIGDADKVAERTKEYEDRFLSPFVAAERGYIDEVIMPHSTRKRIARALGML
RTKEMEQPRKKHDNIPL
Amino acid sequence of GCC M5_L8F (M. extorquens) β-subunit L8F, L100S,
Y143H, D407I, I450V, W502R mutations
SEQ ID NO: 2
MKDILEKFEERRAQARLGGGEKRLEAQHKRGKLTARERIELLLDHGSFEEFDMFVQHRSTDFGMEKQKIP
GDGVVTGWGTVNGRTVFLFSKDFTVFGGSSSEAHAAKIVKVQDMALKMRAPIIGIFDAGGARIQEGVAA
LGGHGEVFRRNVAASGVIPQISVIMGPCAGGDVYSPAMTDFIFMVRDTSYMFVTGPDVVKTVTNEVVT
AEELGGAKVHTSKSSIADGSFENDVEAILQIRRLLDFLPANNIEGVPEIESFDDVNRLDKSLDTLIPDNPNKP
YDMGELIRRVVDEGDFFEIQAAYARNIITGFGRVEGRTVGFVANQPLVLAGVLDSDASRKAARFVRFCNA
FSIPIVTFVDVPGFLPGTAQEYGGLIKHGAKLLFAYSQATVPLVTIITRKAFGGAYIVMASKHVGADLNYAW
PTAQIAVMGAKGAVEIIFRAEIGDADKVAERTKEYEDRFLSPFVAAERGYIDEVIMPHSTRKRIARALGML
RTKEMEQPRKKHDNIPL
Amino acid sequence of GCC M5_G20R (M. extorquens) β-subunit G20R, L100S,
Y143H, D407I, I450V, W502R mutations
SEQ ID NO: 3
MKDILEKLEERRAQARLGGREKRLEAQHKRGKLTARERIELLLDHGSFEEFDMFVQHRSTDFGMEKQKIP
GDGVVTGWGTVNGRTVFLFSKDFTVFGGSSSEAHAAKIVKVQDMALKMRAPIIGIFDAGGARIQEGVAA
LGGHGEVFRRNVAASGVIPQISVIMGPCAGGDVYSPAMTDFIFMVRDTSYMFVTGPDVVKTVTNEVVT
AEELGGAKVHTSKSSIADGSFENDVEAILQIRRLLDFLPANNIEGVPEIESFDDVNRLDKSLDTLIPDNPNKP
YDMGELIRRVVDEGDFFEIQAAYARNIITGFGRVEGRTVGFVANQPLVLAGVLDSDASRKAARFVRFCNA
FSIPIVTFVDVPGFLPGTAQEYGGLIKHGAKLLFAYSQATVPLVTIITRKAFGGAYIVMASKHVGADLNYAW
PTAQIAVMGAKGAVEIIFRAEIGDADKVAERTKEYEDRFLSPFVAAERGYIDEVIMPHSTRKRIARALGML
RTKEMEQPRKKHDNIPL
Amino acid sequence of GCC M5_M53E (M. extorquens) β-subunit M53E, L100S,
Y143H, D407I, I450V, W502R mutations
SEQ ID NO: 4
MKDILEKLEERRAQARLGGGEKRLEAQHKRGKLTARERIELLLDHGSFEEFDEFVQHRSTDFGMEKQKIPG
DGVVTGWGTVNGRTVFLFSKDFTVFGGSSSEAHAAKIVKVQDMALKMRAPIIGIFDAGGARIQEGVAAL
GGHGEVFRRNVAASGVIPQISVIMGPCAGGDVYSPAMTDFIFMVRDTSYMFVTGPDVVKTVTNEVVTA
EELGGAKVHTSKSSIADGSFENDVEAILQIRRLLDFLPANNIEGVPEIESFDDVNRLDKSLDTLIPDNPNKPY
DMGELIRRVVDEGDFFEIQAAYARNIITGFGRVEGRTVGFVANQPLVLAGVLDSDASRKAARFVRFCNAF
SIPIVTFVDVPGFLPGTAQEYGGLIKHGAKLLFAYSQATVPLVTIITRKAFGGAYIVMASKHVGADLNYAWP
TAQIAVMGAKGAVEIIFRAEIGDADKVAERTKEYEDRFLSPFVAAERGYIDEVIMPHSTRKRIARALGMLR
TKEMEQPRKKHDNIPL
Amino acid sequence of GCC M5_M53Q (M. extorquens) β-subunit M53Q, L100S,
Y143H, D407I, I450V, W502R mutations
SEQ ID NO: 5
MKDILEKLEERRAQARLGGGEKRLEAQHKRGKLTARERIELLLDHGSFEEFDQFVQHRSTDFGMEKQKIP
GDGVVTGWGTVNGRTVFLFSKDFTVFGGSSSEAHAAKIVKVQDMALKMRAPIIGIFDAGGARIQEGVAA
LGGHGEVFRRNVAASGVIPQISVIMGPCAGGDVYSPAMTDFIFMVRDTSYMFVTGPDVVKTVTNEVVT
AEELGGAKVHTSKSSIADGSFENDVEAILQIRRLLDFLPANNIEGVPEIESFDDVNRLDKSLDTLIPDNPNKP
YDMGELIRRVVDEGDFFEIQAAYARNIITGFGRVEGRTVGFVANQPLVLAGVLDSDASRKAARFVRFCNA
FSIPIVTFVDVPGFLPGTAQEYGGLIKHGAKLLFAYSQATVPLVTIITRKAFGGAYIVMASKHVGADLNYAW
PTAQIAVMGAKGAVEIIFRAEIGDADKVAERTKEYEDRFLSPFVAAERGYIDEVIMPHSTRKRIARALGML
RTKEMEQPRKKHDNIPL
Amino acid sequence of GCC M5_M64R (M. extorquens) β-subunit M64R, L100S,
Y143H, D407I, I450V, W502R mutations
SEQ ID NO: 6
MKDILEKLEERRAQARLGGGEKRLEAQHKRGKLTARERIELLLDHGSFEEFDMFVQHRSTDFGREKQKIP
GDGVVTGWGTVNGRTVFLFSKDFTVFGGSSSEAHAAKIVKVQDMALKMRAPIIGIFDAGGARIQEGVAA
LGGHGEVFRRNVAASGVIPQISVIMGPCAGGDVYSPAMTDFIFMVRDTSYMFVTGPDVVKTVTNEVVT
AEELGGAKVHTSKSSIADGSFENDVEAILQIRRLLDFLPANNIEGVPEIESFDDVNRLDKSLDTLIPDNPNKP
YDMGELIRRVVDEGDFFEIQAAYARNIITGFGRVEGRTVGFVANQPLVLAGVLDSDASRKAARFVRFCNA
FSIPIVTFVDVPGFLPGTAQEYGGLIKHGAKLLFAYSQATVPLVTIITRKAFGGAYIVMASKHVGADLNYAW
PTAQIAVMGAKGAVEIIFRAEIGDADKVAERTKEYEDRFLSPFVAAERGYIDEVIMPHSTRKRIARALGML
RTKEMEQPRKKHDNIPL
Amino acid sequence of GCC M5_S100N (M. extorquens) β-subunit L100N,
Y143H, D407I, I450V, W502R mutations
SEQ ID NO: 7
MKDILEKLEERRAQARLGGGEKRLEAQHKRGKLTARERIELLLDHGSFEEFDMFVQHRSTDFGMEKQKIP
GDGVVTGWGTVNGRTVFLFSKDFTVFGGSNSEAHAAKIVKVQDMALKMRAPIIGIFDAGGARIQEGVA
ALGGHGEVFRRNVAASGVIPQISVIMGPCAGGDVYSPAMTDFIFMVRDTSYMFVTGPDVVKTVTNEVV
TAEELGGAKVHTSKSSIADGSFENDVEAILQIRRLLDFLPANNIEGVPEIESFDDVNRLDKSLDTLIPDNPNK
PYDMGELIRRVVDEGDFFEIQAAYARNIITGFGRVEGRTVGFVANQPLVLAGVLDSDASRKAARFVRFCN
AFSIPIVTFVDVPGFLPGTAQEYGGLIKHGAKLLFAYSQATVPLVTIITRKAFGGAYIVMASKHVGADLNYA
WPTAQIAVMGAKGAVEIIFRAEIGDADKVAERTKEYEDRFLSPFVAAERGYIDEVIMPHSTRKRIARALGM
LRTKEMEQPRKKHDNIPL
Amino acid sequence of GCC M5_D407V (M. extorquens) β-subunit L100S,
Y143H, D407V, I450V, W502R mutations
SEQ ID NO: 8
MKDILEKLEERRAQARLGGGEKRLEAQHKRGKLTARERIELLLDHGSFEEFDMFVQHRSTDFGMEKQKIP
GDGVVTGWGTVNGRTVFLFSKDFTVFGGSSSEAHAAKIVKVQDMALKMRAPIIGIFDAGGARIQEGVAA
LGGHGEVFRRNVAASGVIPQISVIMGPCAGGDVYSPAMTDFIFMVRDTSYMFVTGPDVVKTVTNEVVT
AEELGGAKVHTSKSSIADGSFENDVEAILQIRRLLDFLPANNIEGVPEIESFDDVNRLDKSLDTLIPDNPNKP
YDMGELIRRVVDEGDFFEIQAAYARNIITGFGRVEGRTVGFVANQPLVLAGVLDSDASRKAARFVRFCNA
FSIPIVTFVDVPGFLPGTAQEYGGLIKHGAKLLFAYSQATVPLVTIITRKAFGGAYVVMASKHVGADLNYA
WPTAQIAVMGAKGAVEIIFRAEIGDADKVAERTKEYEDRFLSPFVAAERGYIDEVIMPHSTRKRIARALGM
LRTKEMEQPRKKHDNIPL
Amino acid sequence of GCC M5_T495N (M. extorquens) β-subunit L100S,
Y143H, D407I, I450V, T495N, W502R mutations
SEQ ID NO: 9
MKDILEKLEERRAQARLGGGEKRLEAQHKRGKLTARERIELLLDHGSFEEFDMFVQHRSTDFGMEKQKIP
GDGVVTGWGTVNGRTVFLFSKDFTVFGGSSSEAHAAKIVKVQDMALKMRAPIIGIFDAGGARIQEGVAA
LGGHGEVFRRNVAASGVIPQISVIMGPCAGGDVYSPAMTDFIFMVRDTSYMFVTGPDVVKTVTNEVVT
AEELGGAKVHTSKSSIADGSFENDVEAILQIRRLLDFLPANNIEGVPEIESFDDVNRLDKSLDTLIPDNPNKP
YDMGELIRRVVDEGDFFEIQAAYARNIITGFGRVEGRTVGFVANQPLVLAGVLDSDASRKAARFVRFCNA
FSIPIVTFVDVPGFLPGTAQEYGGLIKHGAKLLFAYSQATVPLVTIITRKAFGGAYIVMASKHVGADLNYAW
PTAQIAVMGAKGAVEIIFRAEIGDADKVAERTKEYEDRFLSPFVAAERGYIDEVIMPHSTRKRIARALGML
RNKEMEQPRKKHDNIPL
Amino acid sequence of GCC M5_W502H (M. extorquens) β-subunit L100S,
Y143H, D407I, I450V, W502H mutations
SEQ ID NO: 10
MKDILEKLEERRAQARLGGGEKRLEAQHKRGKLTARERIELLLDHGSFEEFDMFVQHRSTDFGMEKQKIP
GDGVVTGWGTVNGRTVFLFSKDFTVFGGSSSEAHAAKIVKVQDMALKMRAPIIGIFDAGGARIQEGVAA
LGGHGEVFRRNVAASGVIPQISVIMGPCAGGDVYSPAMTDFIFMVRDTSYMFVTGPDVVKTVTNEVVT
AEELGGAKVHTSKSSIADGSFENDVEAILQIRRLLDFLPANNIEGVPEIESFDDVNRLDKSLDTLIPDNPNKP
YDMGELIRRVVDEGDFFEIQAAYARNIITGFGRVEGRTVGFVANQPLVLAGVLDSDASRKAARFVRFCNA
FSIPIVTFVDVPGFLPGTAQEYGGLIKHGAKLLFAYSQATVPLVTIITRKAFGGAYIVMASKHVGADLNYAW
PTAQIAVMGAKGAVEIIFRAEIGDADKVAERTKEYEDRFLSPFVAAERGYIDEVIMPHSTRKRIARALGML
RTKEMEQPHKKHDNIPL
Amino acid sequence of GCC M5_L510F (M. extorquens) β-subunit L100S,
Y143H, D407I, I450V, W502R, L510F mutations
SEQ ID NO: 11
MKDILEKLEERRAQARLGGGEKRLEAQHKRGKLTARERIELLLDHGSFEEFDMFVQHRSTDFGMEKQKIP
GDGVVTGWGTVNGRTVFLFSKDFTVFGGSSSEAHAAKIVKVQDMALKMRAPIIGIFDAGGARIQEGVAA
LGGHGEVFRRNVAASGVIPQISVIMGPCAGGDVYSPAMTDFIFMVRDTSYMFVTGPDVVKTVTNEVVT
AEELGGAKVHTSKSSIADGSFENDVEAILQIRRLLDFLPANNIEGVPEIESFDDVNRLDKSLDTLIPDNPNKP
YDMGELIRRVVDEGDFFEIQAAYARNIITGFGRVEGRTVGFVANQPLVLAGVLDSDASRKAARFVRFCNA
FSIPIVTFVDVPGFLPGTAQEYGGLIKHGAKLLFAYSQATVPLVTIITRKAFGGAYIVMASKHVGADLNYAW
PTAQIAVMGAKGAVEIIFRAEIGDADKVAERTKEYEDRFLSPFVAAERGYIDEVIMPHSTRKRIARALGML
RTKEMEQPRKKHDNIPF
Amino acid sequence of PCC WT (M. extorquens) β-subunit
SEQ ID NO: 12
MKDILEKLEERRAQARLGGGEKRLEAQHKRGKLTARERIELLLDHGSFEEFDMFVQHRSTDFGMEKQKIP
GDGVVTGWGTVNGRTVFLFSKDFTVFGGSLSEAHAAKIVKVQDMALKMRAPIIGIFDAGGARIQEGVAA
LGGYGEVFRRNVAASGVIPQISVIMGPCAGGDVYSPAMTDFIFMVRDTSYMFVTGPDVVKTVTNEVVT
AEELGGAKVHTSKSSIADGSFENDVEAILQIRRLLDFLPANNIEGVPEIESFDDVNRLDKSLDTLIPDNPNKP
YDMGELIRRVVDEGDFFEIQAAYARNIITGFGRVEGRTVGFVANQPLVLAGVLDSDASRKAARFVRFCNA
FSIPIVTFVDVPGFLPGTAQEYGGLIKHGAKLLFAYSQATVPLVTIITRKAFGGAYDVMASKHVGADLNYA
WPTAQIAVMGAKGAVEIIFRAEIGDADKIAERTKEYEDRFLSPFVAAERGYIDEVIMPHSTRKRIARALGM
LRTKEMEQPWKKHDNIPL
Amino acid sequence of PCC WT (M. extorquens) α-subunit
SEQ ID NO: 13
MFDKILIANRGEIACRIIKTAQKMGIKTVAVYSDADRDAVHVAMADEAVHIGPAPAAQSYLLIEKIIDACK
QTGAQAVHPGYGFLSERESFPKALAEAGIVFIGPNPGAIAAMGDKIESKKAAAAAEVSTVPGFLGVIESPE
HAVTIADEIGYPVMIKASAGGGGKGMRIAESADEVAEGFARAKSEASSSFGDDRVFVEKFITDPRHIEIQVI
GDKHGNVIYLGERECSIQRRNQKVIEEAPSPLLDEETRRKMGEQAVALAKAVNYDSAGTVEFVAGQDKSF
YFLEMNTRLQVEHPVTEMITGLDLVELMIRVAAGEKLPLSQDQVKLDGWAVESRVYAEDPTRNFLPSIGR
LTTYQPPEEGPLGGAIVRNDTGVEEGGEIAIHYDPMIAKLVTWAPTRLEAIEAQATALDAFAIEGIRHNIPF
LATLMAHPRWRDGRLSTGFIKEEFPEGFIAPEPEGPVAHRLAAVAAAIDHKLNIRKRGISGQMRDPSLLTF
QRERVVVLSGQRFNVTVDPDGDDLLVTFDDGTTAPVRSAWRPGAPVWSGTVGDQSVAIQVRPLLNGV
FLQHAGAAAEARVFTRREAELADLMPVKENAGSGKQLLCPMPGLVKQIMVSEGQEVKNGEPLAIVEAM
KMENVLRAERDGTISKIAAKEGDSLAVDAVILEFA
Amino acid sequence of Biotin ligase BirA (M. extorquens)
SEQ ID NO: 27
MQFRLSQAARSEGHRLHSHDRLDSTNSEAMRLAQGGETGPLWVTTQRQEAGRGRRGNAWTSPEGNL
AASLLMPVAGVAPEMVATLGFVAGVALVDALRDACRLALSPRAEMGDAPLPQGIAPSAAAIHLKWPND
VLADGQKLAGILLEAETLPGGRRAVVVGFGVNVAAAPDGLPYPAAALAAFSAADAPMLLEFLSERFVEAV
RIWNKGRGFSNIRRRWLERAAGVGAPVSVRMAEVTLTGIFETIDEGGRLVILAPDGTRRTVTAGEVHFGS
AATAA
Nucleotide sequences
Nucleotide sequence encoding GCC M5 (M. extorquens) β-subunit L100S,
Y143H, D407I, I450V, W502R mutations
SEQ ID NO: 14
ATGAAGGACATCCTCGAGAAGCTTGAGGAGCGTCGCGCACAGGCCCGTCTCGGCGGCGGGGAAAA
GCGGCTCGAGGCGCAGCACAAGCGCGGCAAGCTCACGGCGCGCGAGCGCATCGAACTCCTGCTCGA
CCACGGGTCGTTCGAGGAGTTCGACATGTTCGTGCAGCACCGCTCCACCGATTTCGGCATGGAGAAG
CAGAAGATCCCCGGCGACGGCGTCGTCACCGGCTGGGGCACCGTGAACGGGCGCACCGTCTTCCTG
TTCTCGAAGGACTTCACGGTGTTCGGCGGCTCGTCCTCCGAGGCGCACGCAGCCAAGATCGTTAAGG
TCCAGGACATGGCGCTGAAGATGCGCGCCCCGATCATCGGCATCTTCGATGCCGGCGGTGCGCGCA
TCCAGGAGGGCGTGGCCGCGCTCGGCGGCCACGGCGAGGTGTTCCGCCGCAACGTCGCTGCCTCCG
GCGTGATCCCGCAGATCTCGGTCATCATGGGGCCGTGCGCGGGCGGCGACGTGTACTCGCCGGCCA
TGACCGACTTCATCTTCATGGTGCGTGACACGAGCTACATGTTCGTGACCGGCCCCGACGTGGTGAA
GACCGTCACCAACGAGGTCGTGACCGCCGAGGAACTCGGCGGCGCCAAGGTCCACACCTCGAAATC
CTCGATCGCCGACGGCTCGTTCGAGAACGACGTCGAGGCGATCCTCCAGATCCGCCGCCTGCTCGAC
TTCCTGCCCGCGAACAACATCGAGGGCGTGCCGGAGATCGAGAGCTTCGACGACGTCAACCGCCTC
GACAAGTCGCTCGACACGCTGATCCCGGACAACCCGAACAAGCCCTACGACATGGGCGAGCTGATC
CGCCGGGTCGTGGACGAAGGCGACTTCTTCGAGATCCAGGCGGCTTACGCCCGCAATATTATCACCG
GCTTCGGCCGCGTCGAGGGCCGCACCGTCGGTTTCGTCGCCAACCAGCCGCTGGTGCTGGCCGGCG
TGCTTGATTCGGACGCCTCCCGGAAGGCGGCCCGCTTCGTGCGCTTCTGCAACGCCTTCTCGATCCCG
ATCGTCACCTTCGTGGACGTGCCGGGCTTCCTGCCGGGCACGGCGCAGGAATATGGCGGCCTGATC
AAGCACGGCGCCAAGCTGCTCTTCGCCTACAGCCAAGCCACCGTGCCGCTCGTGACCATCATCACCC
GCAAGGCCTTCGGCGGCGCCTACATCGTCATGGCCTCCAAGCATGTCGGCGCCGACCTGAACTACGC
GTGGCCGACGGCGCAGATCGCGGTGATGGGCGCCAAGGGCGCTGTCGAGATCATCTTCCGCGCCGA
GATCGGCGATGCGGACAAGGTCGCCGAGCGGACCAAAGAATACGAGGACCGCTTCCTCTCGCCCTT
CGTGGCGGCGGAGCGCGGCTACATCGACGAGGTGATCATGCCCCACTCCACCCGCAAGCGGATCGC
GCGGGCGCTCGGGATGCTGCGCACCAAGGAGATGGAGCAGCCCAGGAAGAAGCACGACAACATCC
CGCTCTGA
Nucleotide sequence encoding GCC M5_L8F (M. extorquens) β-subunit
L8F, L100S, Y143H, D407I, I450V, W502R mutations
SEQ ID NO: 15
ATGAAGGACATCCTCGAGAAGTTTGAGGAGCGTCGCGCACAGGCCCGTCTCGGCGGCGGGGAAAA
GCGGCTCGAGGCGCAGCACAAGCGCGGCAAGCTCACGGCGCGCGAGCGCATCGAACTCCTGCTCGA
CCACGGGTCGTTCGAGGAGTTCGACATGTTCGTGCAGCACCGCTCCACCGATTTCGGCATGGAGAAG
CAGAAGATCCCCGGCGACGGCGTCGTCACCGGCTGGGGCACCGTGAACGGGCGCACCGTCTTCCTG
TTCTCGAAGGACTTCACGGTGTTCGGCGGCTCGTCCTCCGAGGCGCACGCAGCCAAGATCGTTAAGG
TCCAGGACATGGCGCTGAAGATGCGCGCCCCGATCATCGGCATCTTCGATGCCGGCGGTGCGCGCA
TCCAGGAGGGCGTGGCCGCGCTCGGCGGCCACGGCGAGGTGTTCCGCCGCAACGTCGCTGCCTCCG
GCGTGATCCCGCAGATCTCGGTCATCATGGGGCCGTGCGCGGGCGGCGACGTGTACTCGCCGGCCA
TGACCGACTTCATCTTCATGGTGCGTGACACGAGCTACATGTTCGTGACCGGCCCCGACGTGGTGAA
GACCGTCACCAACGAGGTCGTGACCGCCGAGGAACTCGGCGGCGCCAAGGTCCACACCTCGAAATC
CTCGATCGCCGACGGCTCGTTCGAGAACGACGTCGAGGCGATCCTCCAGATCCGCCGCCTGCTCGAC
TTCCTGCCCGCGAACAACATCGAGGGCGTGCCGGAGATCGAGAGCTTCGACGACGTCAACCGCCTC
GACAAGTCGCTCGACACGCTGATCCCGGACAACCCGAACAAGCCCTACGACATGGGCGAGCTGATC
CGCCGGGTCGTGGACGAAGGCGACTTCTTCGAGATCCAGGCGGCTTACGCCCGCAATATTATCACCG
GCTTCGGCCGCGTCGAGGGCCGCACCGTCGGTTTCGTCGCCAACCAGCCGCTGGTGCTGGCCGGCG
TGCTTGATTCGGACGCCTCCCGGAAGGCGGCCCGCTTCGTGCGCTTCTGCAACGCCTTCTCGATCCCG
ATCGTCACCTTCGTGGACGTGCCGGGCTTCCTGCCGGGCACGGCGCAGGAATATGGCGGCCTGATC
AAGCACGGCGCCAAGCTGCTCTTCGCCTACAGCCAAGCCACCGTGCCGCTCGTGACCATCATCACCC
GCAAGGCCTTCGGCGGCGCCTACATCGTCATGGCCTCCAAGCATGTCGGCGCCGACCTGAACTACGC
GTGGCCGACGGCGCAGATCGCGGTGATGGGCGCCAAGGGCGCTGTCGAGATCATCTTCCGCGCCGA
GATCGGCGATGCGGACAAGGTCGCCGAGCGGACCAAAGAATACGAGGACCGCTTCCTCTCGCCCTT
CGTGGCGGCGGAGCGCGGCTACATCGACGAGGTGATCATGCCCCACTCCACCCGCAAGCGGATCGC
GCGGGCGCTCGGGATGCTGCGCACCAAGGAGATGGAGCAGCCCAGGAAGAAGCACGACAACATCC
CGCTCTGA
Nucleotide sequence encoding GCC M5_G20R (M. extorquens) β-subunit
G20R, L100S, Y143H, D407I, I450V, W502R mutations
SEQ ID NO: 16
ATGAAGGACATCCTCGAGAAGCTTGAGGAGCGTCGCGCACAGGCCCGTCTCGGCGGCCGTGAAAAG
CGGCTCGAGGCGCAGCACAAGCGCGGCAAGCTCACGGCGCGCGAGCGCATCGAACTCCTGCTCGAC
CACGGGTCGTTCGAGGAGTTCGACATGTTCGTGCAGCACCGCTCCACCGATTTCGGCATGGAGAAGC
AGAAGATCCCCGGCGACGGCGTCGTCACCGGCTGGGGCACCGTGAACGGGCGCACCGTCTTCCTGT
TCTCGAAGGACTTCACGGTGTTCGGCGGCTCGTCCTCCGAGGCGCACGCAGCCAAGATCGTTAAGGT
CCAGGACATGGCGCTGAAGATGCGCGCCCCGATCATCGGCATCTTCGATGCCGGCGGTGCGCGCAT
CCAGGAGGGCGTGGCCGCGCTCGGCGGCCACGGCGAGGTGTTCCGCCGCAACGTCGCTGCCTCCGG
CGTGATCCCGCAGATCTCGGTCATCATGGGGCCGTGCGCGGGCGGCGACGTGTACTCGCCGGCCAT
GACCGACTTCATCTTCATGGTGCGTGACACGAGCTACATGTTCGTGACCGGCCCCGACGTGGTGAAG
ACCGTCACCAACGAGGTCGTGACCGCCGAGGAACTCGGCGGCGCCAAGGTCCACACCTCGAAATCC
TCGATCGCCGACGGCTCGTTCGAGAACGACGTCGAGGCGATCCTCCAGATCCGCCGCCTGCTCGACT
TCCTGCCCGCGAACAACATCGAGGGCGTGCCGGAGATCGAGAGCTTCGACGACGTCAACCGCCTCG
ACAAGTCGCTCGACACGCTGATCCCGGACAACCCGAACAAGCCCTACGACATGGGCGAGCTGATCC
GCCGGGTCGTGGACGAAGGCGACTTCTTCGAGATCCAGGCGGCTTACGCCCGCAATATTATCACCGG
CTTCGGCCGCGTCGAGGGCCGCACCGTCGGTTTCGTCGCCAACCAGCCGCTGGTGCTGGCCGGCGT
GCTTGATTCGGACGCCTCCCGGAAGGCGGCCCGCTTCGTGCGCTTCTGCAACGCCTTCTCGATCCCGA
TCGTCACCTTCGTGGACGTGCCGGGCTTCCTGCCGGGCACGGCGCAGGAATATGGCGGCCTGATCA
AGCACGGCGCCAAGCTGCTCTTCGCCTACAGCCAAGCCACCGTGCCGCTCGTGACCATCATCACCCG
CAAGGCCTTCGGCGGCGCCTACATCGTCATGGCCTCCAAGCATGTCGGCGCCGACCTGAACTACGCG
TGGCCGACGGCGCAGATCGCGGTGATGGGCGCCAAGGGCGCTGTCGAGATCATCTTCCGCGCCGAG
ATCGGCGATGCGGACAAGGTCGCCGAGCGGACCAAAGAATACGAGGACCGCTTCCTCTCGCCCTTC
GTGGCGGCGGAGCGCGGCTACATCGACGAGGTGATCATGCCCCACTCCACCCGCAAGCGGATCGCG
CGGGCGCTCGGGATGCTGCGCACCAAGGAGATGGAGCAGCCCAGGAAGAAGCACGACAACATCCC
GCTCTGA
Nucleotide sequence encoding GCC M5_M53E (M. extorquens) β-subunit
M53E, L100S, Y143H, D407I, I450V, W502R mutations
SEQ ID NO: 17
ATGAAGGACATCCTCGAGAAGCTTGAGGAGCGTCGCGCACAGGCCCGTCTCGGCGGCGGGGAAAA
GCGGCTCGAGGCGCAGCACAAGCGCGGCAAGCTCACGGCGCGCGAGCGCATCGAACTCCTGCTCGA
CCACGGGTCGTTCGAGGAGTTCGACGAATTCGTGCAGCACCGCTCCACCGATTTCGGCATGGAGAA
GCAGAAGATCCCCGGCGACGGCGTCGTCACCGGCTGGGGCACCGTGAACGGGCGCACCGTCTTCCT
GTTCTCGAAGGACTTCACGGTGTTCGGCGGCTCGTCCTCCGAGGCGCACGCAGCCAAGATCGTTAAG
GTCCAGGACATGGCGCTGAAGATGCGCGCCCCGATCATCGGCATCTTCGATGCCGGCGGTGCGCGC
ATCCAGGAGGGCGTGGCCGCGCTCGGCGGCCACGGCGAGGTGTTCCGCCGCAACGTCGCTGCCTCC
GGCGTGATCCCGCAGATCTCGGTCATCATGGGGCCGTGCGCGGGCGGCGACGTGTACTCGCCGGCC
ATGACCGACTTCATCTTCATGGTGCGTGACACGAGCTACATGTTCGTGACCGGCCCCGACGTGGTGA
AGACCGTCACCAACGAGGTCGTGACCGCCGAGGAACTCGGCGGCGCCAAGGTCCACACCTCGAAAT
CCTCGATCGCCGACGGCTCGTTCGAGAACGACGTCGAGGCGATCCTCCAGATCCGCCGCCTGCTCGA
CTTCCTGCCCGCGAACAACATCGAGGGCGTGCCGGAGATCGAGAGCTTCGACGACGTCAACCGCCTC
GACAAGTCGCTCGACACGCTGATCCCGGACAACCCGAACAAGCCCTACGACATGGGCGAGCTGATC
CGCCGGGTCGTGGACGAAGGCGACTTCTTCGAGATCCAGGCGGCTTACGCCCGCAATATTATCACCG
GCTTCGGCCGCGTCGAGGGCCGCACCGTCGGTTTCGTCGCCAACCAGCCGCTGGTGCTGGCCGGCG
TGCTTGATTCGGACGCCTCCCGGAAGGCGGCCCGCTTCGTGCGCTTCTGCAACGCCTTCTCGATCCCG
ATCGTCACCTTCGTGGACGTGCCGGGCTTCCTGCCGGGCACGGCGCAGGAATATGGCGGCCTGATC
AAGCACGGCGCCAAGCTGCTCTTCGCCTACAGCCAAGCCACCGTGCCGCTCGTGACCATCATCACCC
GCAAGGCCTTCGGCGGCGCCTACATCGTCATGGCCTCCAAGCATGTCGGCGCCGACCTGAACTACGC
GTGGCCGACGGCGCAGATCGCGGTGATGGGCGCCAAGGGCGCTGTCGAGATCATCTTCCGCGCCGA
GATCGGCGATGCGGACAAGGTCGCCGAGCGGACCAAAGAATACGAGGACCGCTTCCTCTCGCCCTT
CGTGGCGGCGGAGCGCGGCTACATCGACGAGGTGATCATGCCCCACTCCACCCGCAAGCGGATCGC
GCGGGCGCTCGGGATGCTGCGCACCAAGGAGATGGAGCAGCCCAGGAAGAAGCACGACAACATCC
CGCTCTGA
Nucleotide sequence encoding GCC M5_M53Q (M. extorquens) β-subunit
M53Q, L100S, Y143H, D407I, I450V, W502R mutations
SEQ ID NO: 18
ATGAAGGACATCCTCGAGAAGCTTGAGGAGCGTCGCGCACAGGCCCGTCTCGGCGGCGGGGAAAA
GCGGCTCGAGGCGCAGCACAAGCGCGGCAAGCTCACGGCGCGCGAGCGCATCGAACTCCTGCTCGA
CCACGGGTCGTTCGAGGAGTTCGACCAGTTCGTGCAGCACCGCTCCACCGATTTCGGCATGGAGAAG
CAGAAGATCCCCGGCGACGGCGTCGTCACCGGCTGGGGCACCGTGAACGGGCGCACCGTCTTCCTG
TTCTCGAAGGACTTCACGGTGTTCGGCGGCTCGTCCTCCGAGGCGCACGCAGCCAAGATCGTTAAGG
TCCAGGACATGGCGCTGAAGATGCGCGCCCCGATCATCGGCATCTTCGATGCCGGCGGTGCGCGCA
TCCAGGAGGGCGTGGCCGCGCTCGGCGGCCACGGCGAGGTGTTCCGCCGCAACGTCGCTGCCTCCG
GCGTGATCCCGCAGATCTCGGTCATCATGGGGCCGTGCGCGGGCGGCGACGTGTACTCGCCGGCCA
TGACCGACTTCATCTTCATGGTGCGTGACACGAGCTACATGTTCGTGACCGGCCCCGACGTGGTGAA
GACCGTCACCAACGAGGTCGTGACCGCCGAGGAACTCGGCGGCGCCAAGGTCCACACCTCGAAATC
CTCGATCGCCGACGGCTCGTTCGAGAACGACGTCGAGGCGATCCTCCAGATCCGCCGCCTGCTCGAC
TTCCTGCCCGCGAACAACATCGAGGGCGTGCCGGAGATCGAGAGCTTCGACGACGTCAACCGCCTC
GACAAGTCGCTCGACACGCTGATCCCGGACAACCCGAACAAGCCCTACGACATGGGCGAGCTGATC
CGCCGGGTCGTGGACGAAGGCGACTTCTTCGAGATCCAGGCGGCTTACGCCCGCAATATTATCACCG
GCTTCGGCCGCGTCGAGGGCCGCACCGTCGGTTTCGTCGCCAACCAGCCGCTGGTGCTGGCCGGCG
TGCTTGATTCGGACGCCTCCCGGAAGGCGGCCCGCTTCGTGCGCTTCTGCAACGCCTTCTCGATCCCG
ATCGTCACCTTCGTGGACGTGCCGGGCTTCCTGCCGGGCACGGCGCAGGAATATGGCGGCCTGATC
AAGCACGGCGCCAAGCTGCTCTTCGCCTACAGCCAAGCCACCGTGCCGCTCGTGACCATCATCACCC
GCAAGGCCTTCGGCGGCGCCTACATCGTCATGGCCTCCAAGCATGTCGGCGCCGACCTGAACTACGC
GTGGCCGACGGCGCAGATCGCGGTGATGGGCGCCAAGGGCGCTGTCGAGATCATCTTCCGCGCCGA
GATCGGCGATGCGGACAAGGTCGCCGAGCGGACCAAAGAATACGAGGACCGCTTCCTCTCGCCCTT
CGTGGCGGCGGAGCGCGGCTACATCGACGAGGTGATCATGCCCCACTCCACCCGCAAGCGGATCGC
GCGGGCGCTCGGGATGCTGCGCACCAAGGAGATGGAGCAGCCCAGGAAGAAGCACGACAACATCC
CGCTCTGA
Nucleotide sequence encoding GCC M5_M64R (M. extorquens) β-subunit
M64R, L100S, Y143H, D407I, I450V, W502R mutations
SEQ ID NO: 19
ATGAAGGACATCCTCGAGAAGCTTGAGGAGCGTCGCGCACAGGCCCGTCTCGGCGGCGGGGAAAA
GCGGCTCGAGGCGCAGCACAAGCGCGGCAAGCTCACGGCGCGCGAGCGCATCGAACTCCTGCTCGA
CCACGGGTCGTTCGAGGAGTTCGACATGTTCGTGCAGCACCGCTCCACCGATTTCGGCCGTGAGAAG
CAGAAGATCCCCGGCGACGGCGTCGTCACCGGCTGGGGCACCGTGAACGGGCGCACCGTCTTCCTG
TTCTCGAAGGACTTCACGGTGTTCGGCGGCTCGTCCTCCGAGGCGCACGCAGCCAAGATCGTTAAGG
TCCAGGACATGGCGCTGAAGATGCGCGCCCCGATCATCGGCATCTTCGATGCCGGCGGTGCGCGCA
TCCAGGAGGGCGTGGCCGCGCTCGGCGGCCACGGCGAGGTGTTCCGCCGCAACGTCGCTGCCTCCG
GCGTGATCCCGCAGATCTCGGTCATCATGGGGCCGTGCGCGGGCGGCGACGTGTACTCGCCGGCCA
TGACCGACTTCATCTTCATGGTGCGTGACACGAGCTACATGTTCGTGACCGGCCCCGACGTGGTGAA
GACCGTCACCAACGAGGTCGTGACCGCCGAGGAACTCGGCGGCGCCAAGGTCCACACCTCGAAATC
CTCGATCGCCGACGGCTCGTTCGAGAACGACGTCGAGGCGATCCTCCAGATCCGCCGCCTGCTCGAC
TTCCTGCCCGCGAACAACATCGAGGGCGTGCCGGAGATCGAGAGCTTCGACGACGTCAACCGCCTC
GACAAGTCGCTCGACACGCTGATCCCGGACAACCCGAACAAGCCCTACGACATGGGCGAGCTGATC
CGCCGGGTCGTGGACGAAGGCGACTTCTTCGAGATCCAGGCGGCTTACGCCCGCAATATTATCACCG
GCTTCGGCCGCGTCGAGGGCCGCACCGTCGGTTTCGTCGCCAACCAGCCGCTGGTGCTGGCCGGCG
TGCTTGATTCGGACGCCTCCCGGAAGGCGGCCCGCTTCGTGCGCTTCTGCAACGCCTTCTCGATCCCG
ATCGTCACCTTCGTGGACGTGCCGGGCTTCCTGCCGGGCACGGCGCAGGAATATGGCGGCCTGATC
AAGCACGGCGCCAAGCTGCTCTTCGCCTACAGCCAAGCCACCGTGCCGCTCGTGACCATCATCACCC
GCAAGGCCTTCGGCGGCGCCTACATCGTCATGGCCTCCAAGCATGTCGGCGCCGACCTGAACTACGC
GTGGCCGACGGCGCAGATCGCGGTGATGGGCGCCAAGGGCGCTGTCGAGATCATCTTCCGCGCCGA
GATCGGCGATGCGGACAAGGTCGCCGAGCGGACCAAAGAATACGAGGACCGCTTCCTCTCGCCCTT
CGTGGCGGCGGAGCGCGGCTACATCGACGAGGTGATCATGCCCCACTCCACCCGCAAGCGGATCGC
GCGGGCGCTCGGGATGCTGCGCACCAAGGAGATGGAGCAGCCCAGGAAGAAGCACGACAACATCC
CGCTCTGA
Nucleotide sequence encoding GCC M5_S100N (M. extorquens) β-subunit
L100N, Y143H, D407I, I450V, W502R mutations
SEQ ID NO: 20
ATGAAGGACATCCTCGAGAAGCTTGAGGAGCGTCGCGCACAGGCCCGTCTCGGCGGCGGGGAAAA
GCGGCTCGAGGCGCAGCACAAGCGCGGCAAGCTCACGGCGCGCGAGCGCATCGAACTCCTGCTCGA
CCACGGGTCGTTCGAGGAGTTCGACATGTTCGTGCAGCACCGCTCCACCGATTTCGGCATGGAGAAG
CAGAAGATCCCCGGCGACGGCGTCGTCACCGGCTGGGGCACCGTGAACGGGCGCACCGTCTTCCTG
TTCTCGAAGGACTTCACGGTGTTCGGCGGCTCGAACTCCGAGGCGCACGCAGCCAAGATCGTTAAG
GTCCAGGACATGGCGCTGAAGATGCGCGCCCCGATCATCGGCATCTTCGATGCCGGCGGTGCGCGC
ATCCAGGAGGGCGTGGCCGCGCTCGGCGGCCACGGCGAGGTGTTCCGCCGCAACGTCGCTGCCTCC
GGCGTGATCCCGCAGATCTCGGTCATCATGGGGCCGTGCGCGGGCGGCGACGTGTACTCGCCGGCC
ATGACCGACTTCATCTTCATGGTGCGTGACACGAGCTACATGTTCGTGACCGGCCCCGACGTGGTGA
AGACCGTCACCAACGAGGTCGTGACCGCCGAGGAACTCGGCGGCGCCAAGGTCCACACCTCGAAAT
CCTCGATCGCCGACGGCTCGTTCGAGAACGACGTCGAGGCGATCCTCCAGATCCGCCGCCTGCTCGA
CTTCCTGCCCGCGAACAACATCGAGGGCGTGCCGGAGATCGAGAGCTTCGACGACGTCAACCGCCTC
GACAAGTCGCTCGACACGCTGATCCCGGACAACCCGAACAAGCCCTACGACATGGGCGAGCTGATC
CGCCGGGTCGTGGACGAAGGCGACTTCTTCGAGATCCAGGCGGCTTACGCCCGCAATATTATCACCG
GCTTCGGCCGCGTCGAGGGCCGCACCGTCGGTTTCGTCGCCAACCAGCCGCTGGTGCTGGCCGGCG
TGCTTGATTCGGACGCCTCCCGGAAGGCGGCCCGCTTCGTGCGCTTCTGCAACGCCTTCTCGATCCCG
ATCGTCACCTTCGTGGACGTGCCGGGCTTCCTGCCGGGCACGGCGCAGGAATATGGCGGCCTGATC
AAGCACGGCGCCAAGCTGCTCTTCGCCTACAGCCAAGCCACCGTGCCGCTCGTGACCATCATCACCC
GCAAGGCCTTCGGCGGCGCCTACATCGTCATGGCCTCCAAGCATGTCGGCGCCGACCTGAACTACGC
GTGGCCGACGGCGCAGATCGCGGTGATGGGCGCCAAGGGCGCTGTCGAGATCATCTTCCGCGCCGA
GATCGGCGATGCGGACAAGGTCGCCGAGCGGACCAAAGAATACGAGGACCGCTTCCTCTCGCCCTT
CGTGGCGGCGGAGCGCGGCTACATCGACGAGGTGATCATGCCCCACTCCACCCGCAAGCGGATCGC
GCGGGCGCTCGGGATGCTGCGCACCAAGGAGATGGAGCAGCCCAGGAAGAAGCACGACAACATCC
CGCTCTGA
Nucleotide sequence encoding GCC M5_D407V (M. extorquens) β-subunit
L100S, Y143H, D407V, I450V, W502R mutations
SEQ ID NO: 21
ATGAAGGACATCCTCGAGAAGCTTGAGGAGCGTCGCGCACAGGCCCGTCTCGGCGGCGGGGAAAA
GCGGCTCGAGGCGCAGCACAAGCGCGGCAAGCTCACGGCGCGCGAGCGCATCGAACTCCTGCTCGA
CCACGGGTCGTTCGAGGAGTTCGACATGTTCGTGCAGCACCGCTCCACCGATTTCGGCATGGAGAAG
CAGAAGATCCCCGGCGACGGCGTCGTCACCGGCTGGGGCACCGTGAACGGGCGCACCGTCTTCCTG
TTCTCGAAGGACTTCACGGTGTTCGGCGGCTCGTCCTCCGAGGCGCACGCAGCCAAGATCGTTAAGG
TCCAGGACATGGCGCTGAAGATGCGCGCCCCGATCATCGGCATCTTCGATGCCGGCGGTGCGCGCA
TCCAGGAGGGCGTGGCCGCGCTCGGCGGCCACGGCGAGGTGTTCCGCCGCAACGTCGCTGCCTCCG
GCGTGATCCCGCAGATCTCGGTCATCATGGGGCCGTGCGCGGGCGGCGACGTGTACTCGCCGGCCA
TGACCGACTTCATCTTCATGGTGCGTGACACGAGCTACATGTTCGTGACCGGCCCCGACGTGGTGAA
GACCGTCACCAACGAGGTCGTGACCGCCGAGGAACTCGGCGGCGCCAAGGTCCACACCTCGAAATC
CTCGATCGCCGACGGCTCGTTCGAGAACGACGTCGAGGCGATCCTCCAGATCCGCCGCCTGCTCGAC
TTCCTGCCCGCGAACAACATCGAGGGCGTGCCGGAGATCGAGAGCTTCGACGACGTCAACCGCCTC
GACAAGTCGCTCGACACGCTGATCCCGGACAACCCGAACAAGCCCTACGACATGGGCGAGCTGATC
CGCCGGGTCGTGGACGAAGGCGACTTCTTCGAGATCCAGGCGGCTTACGCCCGCAATATTATCACCG
GCTTCGGCCGCGTCGAGGGCCGCACCGTCGGTTTCGTCGCCAACCAGCCGCTGGTGCTGGCCGGCG
TGCTTGATTCGGACGCCTCCCGGAAGGCGGCCCGCTTCGTGCGCTTCTGCAACGCCTTCTCGATCCCG
ATCGTCACCTTCGTGGACGTGCCGGGCTTCCTGCCGGGCACGGCGCAGGAATATGGCGGCCTGATC
AAGCACGGCGCCAAGCTGCTCTTCGCCTACAGCCAAGCCACCGTGCCGCTCGTGACCATCATCACCC
GCAAGGCCTTCGGCGGCGCCTACGTCGTCATGGCCTCCAAGCATGTCGGCGCCGACCTGAACTACGC
GTGGCCGACGGCGCAGATCGCGGTGATGGGCGCCAAGGGCGCTGTCGAGATCATCTTCCGCGCCGA
GATCGGCGATGCGGACAAGGTCGCCGAGCGGACCAAAGAATACGAGGACCGCTTCCTCTCGCCCTT
CGTGGCGGCGGAGCGCGGCTACATCGACGAGGTGATCATGCCCCACTCCACCCGCAAGCGGATCGC
GCGGGCGCTCGGGATGCTGCGCACCAAGGAGATGGAGCAGCCCAGGAAGAAGCACGACAACATCC
CGCTCTGA
Nucleotide sequence encoding GCC M5_T495N (M. extorquens) β-subunit
L100S, Y143H, D407I, I450V, T495N, W502R mutations
SEQ ID NO: 22
ATGAAGGACATCCTCGAGAAGCTTGAGGAGCGTCGCGCACAGGCCCGTCTCGGCGGCGGGGAAAA
GCGGCTCGAGGCGCAGCACAAGCGCGGCAAGCTCACGGCGCGCGAGCGCATCGAACTCCTGCTCGA
CCACGGGTCGTTCGAGGAGTTCGACATGTTCGTGCAGCACCGCTCCACCGATTTCGGCATGGAGAAG
CAGAAGATCCCCGGCGACGGCGTCGTCACCGGCTGGGGCACCGTGAACGGGCGCACCGTCTTCCTG
TTCTCGAAGGACTTCACGGTGTTCGGCGGCTCGTCCTCCGAGGCGCACGCAGCCAAGATCGTTAAGG
TCCAGGACATGGCGCTGAAGATGCGCGCCCCGATCATCGGCATCTTCGATGCCGGCGGTGCGCGCA
TCCAGGAGGGCGTGGCCGCGCTCGGCGGCCACGGCGAGGTGTTCCGCCGCAACGTCGCTGCCTCCG
GCGTGATCCCGCAGATCTCGGTCATCATGGGGCCGTGCGCGGGCGGCGACGTGTACTCGCCGGCCA
TGACCGACTTCATCTTCATGGTGCGTGACACGAGCTACATGTTCGTGACCGGCCCCGACGTGGTGAA
GACCGTCACCAACGAGGTCGTGACCGCCGAGGAACTCGGCGGCGCCAAGGTCCACACCTCGAAATC
CTCGATCGCCGACGGCTCGTTCGAGAACGACGTCGAGGCGATCCTCCAGATCCGCCGCCTGCTCGAC
TTCCTGCCCGCGAACAACATCGAGGGCGTGCCGGAGATCGAGAGCTTCGACGACGTCAACCGCCTC
GACAAGTCGCTCGACACGCTGATCCCGGACAACCCGAACAAGCCCTACGACATGGGCGAGCTGATC
CGCCGGGTCGTGGACGAAGGCGACTTCTTCGAGATCCAGGCGGCTTACGCCCGCAATATTATCACCG
GCTTCGGCCGCGTCGAGGGCCGCACCGTCGGTTTCGTCGCCAACCAGCCGCTGGTGCTGGCCGGCG
TGCTTGATTCGGACGCCTCCCGGAAGGCGGCCCGCTTCGTGCGCTTCTGCAACGCCTTCTCGATCCCG
ATCGTCACCTTCGTGGACGTGCCGGGCTTCCTGCCGGGCACGGCGCAGGAATATGGCGGCCTGATC
AAGCACGGCGCCAAGCTGCTCTTCGCCTACAGCCAAGCCACCGTGCCGCTCGTGACCATCATCACCC
GCAAGGCCTTCGGCGGCGCCTACATCGTCATGGCCTCCAAGCATGTCGGCGCCGACCTGAACTACGC
GTGGCCGACGGCGCAGATCGCGGTGATGGGCGCCAAGGGCGCTGTCGAGATCATCTTCCGCGCCGA
GATCGGCGATGCGGACAAGGTCGCCGAGCGGACCAAAGAATACGAGGACCGCTTCCTCTCGCCCTT
CGTGGCGGCGGAGCGCGGCTACATCGACGAGGTGATCATGCCCCACTCCACCCGCAAGCGGATCGC
GCGGGCGCTCGGGATGCTGCGCAACAAGGAGATGGAGCAGCCCAGGAAGAAGCACGACAACATCC
CGCTCTGA
Nucleotide sequence encoding GCC M5_W502H (M. extorquens) β-subunit
L100S, Y143H, D407I, I450V, W502H mutations
SEQ ID NO: 23
ATGAAGGACATCCTCGAGAAGCTTGAGGAGCGTCGCGCACAGGCCCGTCTCGGCGGCGGGGAAAA
GCGGCTCGAGGCGCAGCACAAGCGCGGCAAGCTCACGGCGCGCGAGCGCATCGAACTCCTGCTCGA
CCACGGGTCGTTCGAGGAGTTCGACATGTTCGTGCAGCACCGCTCCACCGATTTCGGCATGGAGAAG
CAGAAGATCCCCGGCGACGGCGTCGTCACCGGCTGGGGCACCGTGAACGGGCGCACCGTCTTCCTG
TTCTCGAAGGACTTCACGGTGTTCGGCGGCTCGTCCTCCGAGGCGCACGCAGCCAAGATCGTTAAGG
TCCAGGACATGGCGCTGAAGATGCGCGCCCCGATCATCGGCATCTTCGATGCCGGCGGTGCGCGCA
TCCAGGAGGGCGTGGCCGCGCTCGGCGGCCACGGCGAGGTGTTCCGCCGCAACGTCGCTGCCTCCG
GCGTGATCCCGCAGATCTCGGTCATCATGGGGCCGTGCGCGGGCGGCGACGTGTACTCGCCGGCCA
TGACCGACTTCATCTTCATGGTGCGTGACACGAGCTACATGTTCGTGACCGGCCCCGACGTGGTGAA
GACCGTCACCAACGAGGTCGTGACCGCCGAGGAACTCGGCGGCGCCAAGGTCCACACCTCGAAATC
CTCGATCGCCGACGGCTCGTTCGAGAACGACGTCGAGGCGATCCTCCAGATCCGCCGCCTGCTCGAC
TTCCTGCCCGCGAACAACATCGAGGGCGTGCCGGAGATCGAGAGCTTCGACGACGTCAACCGCCTC
GACAAGTCGCTCGACACGCTGATCCCGGACAACCCGAACAAGCCCTACGACATGGGCGAGCTGATC
CGCCGGGTCGTGGACGAAGGCGACTTCTTCGAGATCCAGGCGGCTTACGCCCGCAATATTATCACCG
GCTTCGGCCGCGTCGAGGGCCGCACCGTCGGTTTCGTCGCCAACCAGCCGCTGGTGCTGGCCGGCG
TGCTTGATTCGGACGCCTCCCGGAAGGCGGCCCGCTTCGTGCGCTTCTGCAACGCCTTCTCGATCCCG
ATCGTCACCTTCGTGGACGTGCCGGGCTTCCTGCCGGGCACGGCGCAGGAATATGGCGGCCTGATC
AAGCACGGCGCCAAGCTGCTCTTCGCCTACAGCCAAGCCACCGTGCCGCTCGTGACCATCATCACCC
GCAAGGCCTTCGGCGGCGCCTACATCGTCATGGCCTCCAAGCATGTCGGCGCCGACCTGAACTACGC
GTGGCCGACGGCGCAGATCGCGGTGATGGGCGCCAAGGGCGCTGTCGAGATCATCTTCCGCGCCGA
GATCGGCGATGCGGACAAGGTCGCCGAGCGGACCAAAGAATACGAGGACCGCTTCCTCTCGCCCTT
CGTGGCGGCGGAGCGCGGCTACATCGACGAGGTGATCATGCCCCACTCCACCCGCAAGCGGATCGC
GCGGGCGCTCGGGATGCTGCGCACCAAGGAGATGGAGCAGCCCCATAAGAAGCACGACAACATCCC
GCTCTGA
Nucleotide sequence encoding GCC M5_L510F (M. extorquens) β-subunit
L100S, Y143H, D407I, I450V, W502R, L510F mutations
SEQ ID NO: 24
ATGAAGGACATCCTCGAGAAGCTTGAGGAGCGTCGCGCACAGGCCCGTCTCGGCGGCGGGGAAAA
GCGGCTCGAGGCGCAGCACAAGCGCGGCAAGCTCACGGCGCGCGAGCGCATCGAACTCCTGCTCGA
CCACGGGTCGTTCGAGGAGTTCGACATGTTCGTGCAGCACCGCTCCACCGATTTCGGCATGGAGAAG
CAGAAGATCCCCGGCGACGGCGTCGTCACCGGCTGGGGCACCGTGAACGGGCGCACCGTCTTCCTG
TTCTCGAAGGACTTCACGGTGTTCGGCGGCTCGTCCTCCGAGGCGCACGCAGCCAAGATCGTTAAGG
TCCAGGACATGGCGCTGAAGATGCGCGCCCCGATCATCGGCATCTTCGATGCCGGCGGTGCGCGCA
TCCAGGAGGGCGTGGCCGCGCTCGGCGGCCACGGCGAGGTGTTCCGCCGCAACGTCGCTGCCTCCG
GCGTGATCCCGCAGATCTCGGTCATCATGGGGCCGTGCGCGGGCGGCGACGTGTACTCGCCGGCCA
TGACCGACTTCATCTTCATGGTGCGTGACACGAGCTACATGTTCGTGACCGGCCCCGACGTGGTGAA
GACCGTCACCAACGAGGTCGTGACCGCCGAGGAACTCGGCGGCGCCAAGGTCCACACCTCGAAATC
CTCGATCGCCGACGGCTCGTTCGAGAACGACGTCGAGGCGATCCTCCAGATCCGCCGCCTGCTCGAC
TTCCTGCCCGCGAACAACATCGAGGGCGTGCCGGAGATCGAGAGCTTCGACGACGTCAACCGCCTC
GACAAGTCGCTCGACACGCTGATCCCGGACAACCCGAACAAGCCCTACGACATGGGCGAGCTGATC
CGCCGGGTCGTGGACGAAGGCGACTTCTTCGAGATCCAGGCGGCTTACGCCCGCAATATTATCACCG
GCTTCGGCCGCGTCGAGGGCCGCACCGTCGGTTTCGTCGCCAACCAGCCGCTGGTGCTGGCCGGCG
TGCTTGATTCGGACGCCTCCCGGAAGGCGGCCCGCTTCGTGCGCTTCTGCAACGCCTTCTCGATCCCG
ATCGTCACCTTCGTGGACGTGCCGGGCTTCCTGCCGGGCACGGCGCAGGAATATGGCGGCCTGATC
AAGCACGGCGCCAAGCTGCTCTTCGCCTACAGCCAAGCCACCGTGCCGCTCGTGACCATCATCACCC
GCAAGGCCTTCGGCGGCGCCTACATCGTCATGGCCTCCAAGCATGTCGGCGCCGACCTGAACTACGC
GTGGCCGACGGCGCAGATCGCGGTGATGGGCGCCAAGGGCGCTGTCGAGATCATCTTCCGCGCCGA
GATCGGCGATGCGGACAAGGTCGCCGAGCGGACCAAAGAATACGAGGACCGCTTCCTCTCGCCCTT
CGTGGCGGCGGAGCGCGGCTACATCGACGAGGTGATCATGCCCCACTCCACCCGCAAGCGGATCGC
GCGGGCGCTCGGGATGCTGCGCACCAAGGAGATGGAGCAGCCCAGGAAGAAGCACGACAACATCC
CGTTCTGA
Nucleotide sequence encoding PCC WT (M. extorquens) β-subunit
SEQ ID NO: 25
ATGAAGGACATTCTCGAGAAGCTTGAGGAGCGTCGCGCACAGGCCCGTCTCGGCGGCGGGGAAAA
GCGGCTCGAGGCGCAGCACAAGCGCGGCAAGCTCACGGCGCGCGAGCGCATCGAACTCCTGCTCGA
CCACGGGTCGTTCGAGGAGTTCGACATGTTCGTGCAGCACCGCTCCACCGATTTCGGCATGGAGAAG
CAGAAGATCCCCGGCGACGGCGTCGTCACCGGCTGGGGCACCGTGAACGGGCGCACCGTCTTCCTG
TTCTCGAAGGACTTCACGGTGTTCGGCGGCTCGCTCTCCGAGGCGCACGCAGCCAAGATCGTTAAGG
TCCAGGACATGGCGCTGAAGATGCGCGCCCCGATCATCGGCATCTTCGATGCCGGCGGTGCGCGCA
TCCAGGAGGGCGTGGCCGCGCTCGGCGGCTACGGCGAGGTGTTCCGCCGCAACGTCGCTGCCTCCG
GCGTGATCCCGCAGATCTCGGTCATCATGGGGCCGTGCGCGGGCGGCGACGTGTACTCGCCGGCCA
TGACCGACTTCATCTTCATGGTGCGTGACACGAGCTACATGTTCGTGACCGGCCCCGACGTGGTGAA
GACCGTCACCAACGAGGTCGTGACCGCCGAGGAACTCGGCGGCGCCAAGGTCCACACCTCGAAATC
CTCGATCGCCGACGGCTCGTTCGAGAACGACGTCGAGGCGATCCTCCAGATCCGCCGCCTGCTCGAC
TTCCTGCCCGCGAACAACATCGAGGGCGTGCCGGAGATCGAGAGCTTCGACGACGTCAACCGCCTC
GACAAGTCGCTCGACACGCTGATCCCGGACAACCCGAACAAGCCCTACGACATGGGCGAGCTGATC
CGCCGGGTCGTGGACGAAGGCGACTTCTTCGAGATCCAGGCGGCTTACGCCCGCAATATTATCACCG
GCTTCGGCCGCGTCGAGGGCCGCACCGTCGGTTTCGTCGCCAACCAGCCGCTGGTGCTGGCCGGCG
TGCTTGATTCGGACGCCTCCCGGAAGGCGGCCCGCTTCGTGCGCTTCTGCAACGCCTTCTCGATCCCG
ATCGTCACCTTCGTGGACGTGCCGGGCTTCCTGCCGGGCACGGCGCAGGAATATGGCGGCCTGATC
AAGCACGGCGCCAAGCTGCTCTTCGCCTACAGCCAAGCCACCGTGCCGCTCGTGACCATCATCACCC
GCAAGGCCTTCGGCGGCGCCTACGACGTCATGGCCTCCAAGCATGTCGGCGCCGACCTGAACTACGC
GTGGCCGACGGCGCAGATCGCGGTGATGGGCGCCAAGGGCGCTGTCGAGATCATCTTCCGCGCCGA
GATCGGCGATGCGGACAAGATCGCCGAGCGGACCAAAGAATACGAGGACCGCTTCCTCTCGCCCTT
CGTGGCGGCGGAGCGCGGCTACATCGACGAGGTGATCATGCCCCACTCCACCCGCAAGCGGATCGC
GCGGGCGCTCGGGATGCTGCGCACCAAGGAGATGGAGCAGCCCTGGAAGAAGCACGACAACATCC
CGCTCTGA
Nucleotide sequence encoding PCC WT (M. extorquens) α-subunit
SEQ ID NO: 26
ATGTTCGATAAGATCCTGATTGCCAACCGGGGCGAAATCGCCTGCCGTATCATCAAGACGGCCCAGA
AAATGGGCATCAAGACGGTGGCGGTCTATTCGGACGCCGACCGTGATGCGGTCCACGTCGCGATGG
CCGACGAGGCGGTGCATATCGGCCCGGCGCCCGCTGCGCAGTCCTACCTTCTGATCGAAAAGATCAT
CGACGCCTGCAAGCAGACCGGCGCCCAAGCGGTCCATCCGGGCTACGGCTTCCTTTCCGAGCGCGA
GTCCTTCCCCAAGGCGCTGGCGGAAGCGGGCATCGTCTTTATCGGCCCCAATCCGGGTGCCATCGCC
GCAATGGGCGACAAGATCGAATCGAAGAAGGCCGCGGCCGCGGCCGAGGTCTCGACGGTGCCGGG
CTTCCTCGGCGTGATCGAGAGCCCCGAGCACGCCGTGACGATCGCCGATGAGATCGGCTATCCGGT
GATGATCAAGGCGTCGGCGGGCGGCGGCGGCAAGGGTATGCGCATCGCCGAATCGGCCGATGAGG
TCGCCGAGGGCTTCGCCCGCGCCAAGTCCGAGGCCTCGTCCTCCTTCGGCGACGACCGCGTCTTCGT
GGAAAAGTTCATCACCGACCCGCGCCACATCGAGATCCAGGTGATCGGCGATAAGCACGGCAACGT
GATCTATCTCGGTGAGCGCGAGTGCTCGATCCAGCGCCGCAACCAGAAGGTCATCGAGGAGGCGCC
GTCGCCGCTCCTCGACGAAGAGACGCGCCGCAAGATGGGCGAGCAGGCGGTCGCGCTCGCCAAGG
CCGTGAATTACGACTCCGCCGGCACCGTCGAGTTCGTCGCCGGCCAGGACAAGTCGTTCTACTTCCTC
GAAATGAACACCCGCCTGCAGGTGGAGCACCCGGTCACCGAGATGATCACCGGGCTCGACCTCGTC
GAGCTGATGATCCGGGTGGCCGCCGGCGAGAAGCTGCCGCTGTCGCAGGATCAGGTGAAGCTCGA
CGGCTGGGCGGTCGAGAGCCGCGTCTATGCCGAGGATCCGACCCGCAACTTCCTGCCCTCGATCGGT
CGGCTGACTACCTACCAGCCGCCGGAGGAGGGCCCGCTCGGCGGGGCGATCGTGCGCAACGATACC
GGCGTGGAGGAGGGCGGCGAGATCGCGATCCACTACGATCCGATGATTGCCAAGCTCGTAACCTGG
GCGCCGACCCGGTTGGAAGCCATCGAAGCGCAGGCGACCGCGCTCGACGCCTTCGCCATCGAGGGC
ATCCGCCACAACATCCCCTTCCTCGCCACCCTGATGGCCCATCCCCGCTGGCGCGACGGCCGGCTCTC
GACGGGCTTCATCAAGGAAGAGTTCCCCGAAGGCTTCATCGCACCCGAGCCCGAGGGGCCGGTCGC
TCATCGGCTCGCGGCGGTGGCGGCGGCGATCGATCACAAGCTCAACATCCGCAAGCGCGGCATCTC
CGGCCAGATGCGCGACCCGAGCCTGCTGACCTTCCAGCGCGAGCGCGTGGTGGTGCTCTCCGGCCA
GCGCTTCAACGTCACCGTCGATCCTGACGGCGACGACCTCCTCGTCACCTTCGACGACGGTACGACA
GCCCCGGTGCGCAGCGCGTGGCGCCCCGGTGCGCCGGTCTGGAGCGGTACGGTCGGAGATCAGTC
GGTCGCGATCCAGGTGCGTCCGCTCCTCAACGGTGTGTTCCTGCAGCATGCGGGCGCGGCGGCGGA
AGCGCGGGTGTTCACCCGCCGCGAGGCCGAACTCGCCGACCTGATGCCGGTCAAGGAGAATGCCGG
CTCCGGCAAGCAGCTCCTTTGCCCGATGCCCGGCCTGGTCAAGCAGATCATGGTCAGCGAGGGCCA
GGAGGTGAAGAACGGCGAGCCGCTGGCCATCGTCGAGGCGATGAAGATGGAGAACGTGCTGCGCG
CCGAACGCGACGGCACCATCTCCAAGATCGCCGCCAAGGAAGGCGACAGCCTCGCCGTCGATGCCG
TGATCCTGGAATTCGCCTGA
Nucleotide sequence encoding Biotin ligase BirA (M. extorquens)
SEQ ID NO: 28
ATGCAGTTCCGGCTAAGTCAGGCGGCCCGGTCCGAGGGGCATCGGCTCCACAGCCACGACCGGCTC
GACTCGACCAACAGCGAGGCCATGCGCCTCGCCCAGGGCGGCGAGACGGGCCCGCTCTGGGTCACG
ACCCAACGCCAGGAGGCGGGCCGCGGCCGACGCGGCAACGCCTGGACCTCGCCGGAGGGCAACCT
CGCCGCGAGCCTGCTGATGCCGGTGGCCGGCGTGGCGCCGGAGATGGTGGCCACGCTGGGCTTCGT
GGCGGGCGTGGCGTTGGTGGACGCTCTGCGCGATGCGTGCCGTTTAGCCCTCTCCCCGCGTGCGGA
GATGGGGGATGCACCGCTGCCGCAGGGCATTGCTCCTAGCGCTGCCGCCATCCATCTGAAATGGCCC
AACGACGTCCTCGCCGACGGCCAAAAACTCGCCGGCATCCTGCTGGAGGCCGAGACGCTGCCGGGC
GGGCGGCGGGCGGTGGTGGTCGGTTTCGGCGTCAACGTCGCGGCGGCGCCGGACGGTTTGCCCTA
TCCGGCGGCGGCGCTTGCGGCTTTTTCCGCGGCGGATGCGCCGATGCTGCTCGAATTCCTGTCGGAA
CGGTTTGTCGAAGCCGTCCGGATCTGGAACAAGGGGCGCGGATTCTCGAATATCCGGCGGCGGTGG
CTGGAGCGCGCGGCGGGGGTAGGGGCCCCCGTGTCGGTTCGCATGGCCGAAGTCACGCTGACGGG
CATCTTTGAAACGATCGACGAGGGGGGGCGGCTCGTGATCCTCGCCCCGGACGGAACCCGACGAAC
CGTGACGGCGGGCGAGGTGCATTTCGGAAGTGCCGCGACGGCGGCCTGA

The present invention is further described by reference to the following non-limiting figures and examples.

### BRIEF DESCRIPTION OF THE DRAWINGS

FIG. 1: Activity of mutant variants: glycolyl-CoA carboxylation

Glycolyl-CoA carboxylation rates were measured by spectrophotometric measurements using CaMCR as coupling enzyme. Error bars indicate standard deviation. All measurements were done in triplicates. M5: GCC M5, G20R: mutant variant G20R, S100N: mutant variant S100N (identical to L100N, as used herein).

FIG. 2: Activity of mutant variants: ATP/CO₂-ratio

Ratios between ATP consumption and glycolyl-CoA carboxylation were measured by spectrophotometric measurements using CaMCR as coupling enzyme under ATP-limited conditions. Error bars indicate standard deviation. All measurements were done in triplicates. M5: GCC M5, G20R: mutant variant G20R, S100N: mutant variant S100N (identical to L100N, as used herein).

FIG. 3: Sequence alignment between PCC, GCC-M5 and mutant variants G20R and S100N

Sequence alignments between PCC, GCC M5 and mutant variants G20R and S100N (identical to L100N, as used herein) were generated using Clustal Omega (version 1.2.4). GCC_M5 bears the mutations (versus PCC) L100S(identical to the term S100S), Y143H, D407I, I450V, and W502R (highlighted). GCC_M5_G20R bears the M5 mutations (highlighted) and additionally G20R (bold, highlighted). GCC_M5_S100N bears the M5 mutations (highlighted) and additionally S100N (bold, highlighted). Asterisk (*) indicates positions with fully conserved residues, Colon (:) indicates positions with unconserved but strongly similar properties (scoring >0.5 in the Gonnet PAM 250 matrix).

FIG. 4. Lysate-based screen of site-saturation libraries. Library M5-G20X contains GCC M5 variants with substitutions at position 20, library M5-S100X contains GCC M5 variants with substitutions at position 100 (see method section for detailed description of library construction). Filled dots represent samples without significant activity that were not considered for further analysis. Empty triangles, squares and circles represent samples with activity bearing the residues G20R (triangles), S100N (squares) or S100S (circles) in the background of GCC M5. Accordingly, S100S corresponds to the GCC M5. The initial slope of absorbance at 340 nm decrease during the first 500 s of the reaction is plotted on the y-axis and indicative for the carboxylation rate. In total, 367 samples including 324 mutagenized variants, 20 positive controls with GCC M5-G20R lacking any additional mutations (apart from the G20R mutation as compared to the GCC M5), 15 positive controls with GCC M5-S100N lacking any additional mutations (apart from the S100N mutation as compared to the GCC M5) and 8 negative controls with lysis buffer (CelLytic B; Sigma Aldrich) were measured. As the assay is based on cell-lysates and the GCC M5 protein load was not quantified/standardized, the assay primarily provides qualitative results. The assay indicates that only S100S, S100N, and G20R comprise relevant levels of GCC activity, all other tested substitutions at positions 20 and 100 were comparable to the negative controls.

FIG. 5. Structural analysis of the new GCC MS variants G20R and S100N. A) and B) show surface representations of the cryo-EM electron density maps for the G20R (EMD-17777) and S100N (EMD-17778) variants, respectively. The β-subunits are depicted in dark gray tones, whereas the partial electron densities for the α-subunits are colored in light gray. C) The location of the G20R substitution (PDB 8PN7) is shown on the surface of the β-subunit core (left panel) and a close-up shows the position of Arg20 in proximity of the binding site for the adenosyl-moiety of glycolyl-CoA (right panel). D) Close-up of S100N variant active site (PDB 8PN8) showing the environment of His143 and its putative interaction with glycolyl-CoA. Glycolyl-CoA was modeled corresponding to methylmalonyl-CoA in PDB 1ON3 with additional manual fitting that reflects the binding of CoA in the cryo-EM structures. A manually fitted carboxybiotin is shown in its most likely position for carboxyl transfer to the substrate. His143 engages in polar interactions with the glycolyl-CoA and Asp171. The amide group of S100N is positioned parallel to the imidazole ring of His143 at a distance of 3.9 Å.

FIG. 6. Mass Photometry analysis of the new GCC variants. Mass photometry (MP) data are shown for GCC M5 (A), the GCC M5 G20R variant (B), the GCC M5 S100N variant (C) and PCC from M. extorquens (D). Each peak reflects the absolute number of protein complexes of a certain size, therefore the graphs show the distribution of different protein complexes per sample. All variants show a wide distribution of complexes with differing numbers of α-subunits attached to the β₆-core, underscoring a transient interaction of the subunits. The G20R variant appears to favor the formation of β₆α₆ complexes, as the peak for this formation is bigger than the other peaks. Variant S100N appears to slightly favor the β₆α₆ complex formation in comparison to GCC M5. PCC wildtype shows no clear favorability for any one oligomeric state. All samples show a peak at ~70 kDa representing inactive, monomeric β-subunits.

FIG. 7: Efficiency of the Tartronyl-CoA (TaCo) pathway with different GCC variants compared to native plant photorespiration. Relative theoretical yields were calculated by comparing the amount of NADPH and ATP required to produce 3-phosphoglycerate (3PG) from three molecules of CO₂ (see Methods, Table 9). Oxygenation was assumed to occur in 25% of RuBisCO reactions (Fu, et al., 2023, Nature plants, 9(1), 169-178; Walker, loc.cit.). NPR=Natural plant photorespiration (as occurring during oxygenic photosynthesis), TaCo (M5)=TaCo pathway with GCC M5, TaCo (M5 S100N)=TaCo pathway with GCC M5 S100N, TaCo (theoret.)=theoretically optimal TaCo pathway in which all ATP hydrolysis contributes to 3PG production.

### EXAMPLES

### Materials & Methods

### Materials

Chemicals were obtained from Sigma-Aldrich, Carl Roth GmbH+Co. KG, Santa Cruz Biotechnology Inc. and Merck. Biochemicals and materials for cloning and protein expression were obtained from Thermo Fisher Scientific, New England Biolabs GmbH and Macherey-Nagel GmbH. Coenzyme A was bought from Roche Diagnostics. Materials and equipment for protein purification were obtained from GE Healthcare, BioRad and Merck Millipore GmbH. Pyruvate Kinase/Lactic Dehydrogenase, Malic Dehydrogenase, Glucose-6-Phosphate Dehydrogenase, Glucose Dehydrogenase and Phosphoenolpyruvate carboxylase were bought from Sigma-Aldrich.

### Strains

All strains used in this work are listed in the following table.

TABLE 2
Strains

| Strain | Genotype/Phenotype | Reference |
|---|---|---|
| E. coli NEB Turbo | F^{T} proA⁺B⁺ lacl^{q} ΔlacZM15/fhuA2 Δ(lac- | NEB |
| | proAB) glnV galK16 galE15 R(zgb-210::Tn10)Tet^{S} | |
| | endA1 thi-1 Δ(hsdS-mcrB)5 | |
| E. coli BL21 DE3 | fhuA2 [Ion] ompT gal (λ DE3) [dcm] ΔhsdS | NEB |
| | λ DE3 = λ sBamHlo ΔEcoRI-B | |
| | int::(lacl::PlacUV5::T7 gene1) i21 Δnin5 | |
| E. coli BL21-birA | fhuA2 [Ion] ompT gal (λ DE3) [dcm] ΔhsdS | Scheffen, |
| | λ DE3 = λ sBamHlo ΔEcoRI-B | loc. cit. |
| | int::(lacl::PlacUV5::T7 gene1) i21 Δnin5 amp^{R} harbors | |
| | JZ150 with birA from M. extorquens | |
| E. coli ElectroMAX | F⁻ Φ80lacZΔM15 Δ(lacZYA-argF) U169 recA1 | Invitrogen |
| DH5α | endA1 hsdR17 | |
| | (rₖ⁻, mₖ⁺) gal⁻ phoA supE44 λ⁻ thi⁻1 gyrA96 relA1 | |

E. coli ElectroMAX DH5a was used to create random mutagenesis libraries that were needed to produce a dataset of randomly mutagenized GCC variants to train an artificial intelligence model for the prediction of beneficial mutations. E. coli NEB Turbo was used to construct and maintain plasmids with site-specific mutations in the gene for GCC. E. coli BL21-birA was derived from E. coli BL21 DE3 by introducing a vector that bears a biotin ligase gene from Methylorubrum extorquens that is required to activate GCC. E. coli BL21-birA was used for protein overexpression of GCC variants.

### Plasmids

All plasmids used in this work are listed in the following table.

TABLE 3
Plasmids

| Plasmid | Description | Reference |
|---|---|---|
| pTE3101 | Glycolyl-CoA Carboxylase M5, adapted from M. | Scheffen, loc. cit. |
| | extorquens PCC, codon optimized for E. coli | |
| pTE3142 | Glycolyl-CoA Carboxylase M5_L8F, adapted from | This work |
| | M. extorquens PCC, codon optimized for E. coli | |
| pTE3143 | Glycolyl-CoA Carboxylase M5_G20R, adapted from | This work |
| | M. extorquens PCC, codon optimized for E. coli | |
| pTE3144 | Glycolyl-CoA Carboxylase M5_M53E, adapted from | This work |
| | M. extorquens PCC, codon optimized for E. coli | |
| pTE3145 | Glycolyl-CoA Carboxylase M5_M53Q, adapted from | This work |
| | M. extorquens PCC, codon optimized for E. coli | |
| pTE3146 | Glycolyl-CoA Carboxylase M5_M64R, adapted from | This work |
| | M. extorquens PCC, codon optimized for E. coli | |
| pTE3147 | Glycolyl-CoA Carboxylase M5_S100N, adapted | This work |
| | from M. extorquens PCC, codon optimized for E. | |
| | coli | |
| pTE3148 | Glycolyl-CoA Carboxylase M5_D407V, adapted | This work |
| | from M. extorquens PCC, codon optimized for E. | |
| | coli | |
| pTE3149 | Glycolyl-CoA Carboxylase M5_T495N, adapted | This work |
| | from M. extorquens PCC, codon optimized for E. | |
| | coli | |
| pTE3150 | Glycolyl-CoA Carboxylase M5_W502H, adapted | This work |
| | from M. extorquens PCC, codon optimized for E. | |
| | coli | |
| pTE3151 | Glycolyl-CoA Carboxylase M5_L510F, adapted from | This work |
| | M. extorquens PCC, codon optimized for E. coli | |
| pTrc-McrCa | Malonyl-CoA Reductase from Chloroflexus | Kroeger, Anal. |
| | aurantiacus, codon optimized for E. coli | Biochem. 411, |
| | | 100-105, 2011 |

### Oligonucleotides

All oligonucleotides used in this work are listed in the following table.

TABLE 4
Oligonucleotides

| Oligo | Description | Sequence (5′ → 3′) |
|---|---|---|
| oDM0088 | Sequencing of GCC constructs | cgtccacgaaggtgacgatcg |
| oDM0115 | GCC M5 mutagenesis L8F (ctt to ttt) | ccatatgaaggacatcctcgagaagttt |
| | | gaggagcgtcgcgcacagg |
| oDM0116 | GCC M5 mutagenesis G20R (ggg to cgt) | gcacaggcccgtctcggcggccgtgaa |
| | | aagcggctcgagg |
| oDM0117 | GCC M5 mutagenesis M53E (atg to gaa) | cacgggtcgttcgaggagttcgacgaa |
| | | ttcgtgcagcaccgctccaccg |
| oDM0118 | GCC M5 mutagenesis M53Q (atg to cag) | cacgggtcgttcgaggagttcgaccagt |
| | | tcgtgcagcaccgctccaccg |
| oDM0119 | GCC M5 mutagenesis M64R (atg to cgt) | gcagcaccgctccaccgatttcggccgt |
| | | gagaagcagaagatccccggcg |
| oDM0120 | GCC M5 mutagenesis S100N (tcc to aac) | cacggtgttcggcggctcgaactccgag |
| | | gcgcacgcagccaag |
| oDM0121 | GCC M5 mutagenesis I407V (atc to gtc) | caaggccttcggcggcgcctacgtcgtc |
| | | atggcctccaagcatgtcggcg |
| oDM0122 | GCC M5 mutagenesis T495N (acc to aac) | gggcgctcgggatgctgcgcaacaagg |
| | | agatggagcagcccagg |
| oDM0123 | GCC M5 mutagenesis R502H (agg to cat) | gcaccaaggagatggagcagccccat |
| | | aagaagcacgacaacatcccgctc |
| oDM0124 | GCC M5 mutagenesis L510F (ctc to ttc) | cccaggaagaagcacgacaacatccc |
| | | gttctgagaattcgagctccgtcg |
| oDM0143 | GCC M5 mutagenesis G20A (ggg to gcg) | gcacaggcccgtctcggcggcgcggaa |
| | | aagcggctcgagg |
| oDM0144 | GCC M5 mutagenesis G20H (ggg to cat) | gcacaggcccgtctcggcggccatgaa |
| | | aagcggctcgagg |
| oDM0145 | GCC M5 mutagenesis G20K (ggg to aaa) | gcacaggcccgtctcggcggcaaagaa |
| | | aagcggctcgagg |
| oDM0146 | GCC M5 mutagenesis G20Y (ggg to tat) | gcacaggcccgtctcggcggctatgaa |
| | | aagcggctcgagg |
| oDM0164 | GCC M5 G20X site saturation, single oligo PCR, | gcacaggcccgtctcggcggcndtgaa |
| | primer 1 | aagcggctcgagg |
| oDM0165 | GCC M5 G20X site saturation, single oligo PCR, | gcacaggcccgtctcggcggcvhggaa |
| | primer 2 | aagcggctcgagg |
| oDM0166 | GCC M5 G20X site saturation, single oligo PCR, | gcacaggcccgtctcggcggctgggaa |
| | primer 3 | aagcggctcgagg |
| oDM0167 | GCC M5 L100X site saturation, single oligo PCR, | cacggtgttcggcggctcgndttccgag |
| | primer 1 | gcgcacgcagccaag |
| oDM0168 | GCC M5 L100X site saturation, single oligo PCR, | cacggtgttcggcggctcgvhgtccga |
| | primer 2 | ggcgcacgcagccaag |
| oDM0169 | GCC M5 L100X site saturation, single oligo PCR, | cacggtgttcggcggctcgttgtccgag |
| | primer 3 | gcgcacgcagccaag |
| PCC_seq1 | Sequencing of GCC constructs | gagcggataacaattcccctg |
| PCC_seq2_II | Sequencing of GCC constructs | ccaggacatggcgctgaag |
| PCC_seq3_II | Sequencing of GCC constructs | gaaggcgacttcttcgagatc |
| PCC_seq4 | Sequencing of GCC constructs | gatgctgcgcaccaag |
| PCC_seq5 | Sequencing of GCC constructs | caatccgggtgccatc |
| PCC_seq6 | Sequencing of GCC constructs | gaacacccgcctgcag |
| PCC_seq7 | Sequencing of GCC constructs | cgatcgatcacaagctcaac |
| PccB_fw_P1 | Random mutagenesis primer | gtttaactttaataaggagatataccat |
| | | gggcagcagccatc |
| PccB_rv_P1 | Random mutagenesis primer | gattactttctgttcgacttaagcattat |
| | | gcggccgcaag |
| oDM0170 | GCC M5 G20X library site saturation primer 4 | gcgacgctcctcaagcttc |
| oDM0171 | GCC M5 S100X library site saturation primer 4 | aagtccttcgagaacaggaagac |

### Synthesis of CoA Esters

GCC catalyzes the carboxylation of glycolyl-CoA to tartronyl-CoA. To measure this reaction, glycolyl-CoA was synthesized and purified as previously described (Trudeau, loc. cit.; Scheffen, loc. cit.). The concentration of CoA-esters was quantified by determining the absorption at 260 nm (ε=16.4 mM⁻¹ cm⁻¹).

### Random Mutagenesis Library Generation

In order to produce a dataset to feed the AI algorithm, random mutagenesis libraries of GCC M5 were constructed. Plasmid libraries of randomly mutagenized GCC M5 were created by mega primer-based whole-plasmid PCR (MEGAWHOP) (Miyazaki, loc. cit.). To generate randomized fragments of the β subunit of GCC M5 (pTE3101), error-prone PCR was performed using 2.5 U Taq-polymerase with Mg-free buffer (New England Biolabs; M0320), 7 mM MgCl₂, 0.4 mM dGTP and dATP each, 2 mM dCTP and dTTP each, 0.4 μM primer PccB_fw_P1 and primer PccB_rv_P1 each, 10% (v/v) dimethyl sulfoxide, 50 ng template DNA of pTE3101, and 200-500 μM MnCl₂ in a 50 μL reaction. The randomized fragments were digested with DpnI (NEB, R0176), purified by agarose gel electrophoresis, and used as mega primers for a whole-plasmid PCR (MEGAWHOP), as described elsewhere (Miyazaki, loc. cit.), or subjected to another error-prone PCR reaction to further increase the mutation rate. The MEGAWHOP reaction (50 μL) contained 1×KOD Hot Start reaction buffer (Novagen), 0.2 mM dNTPs, 1.5 mM MgSO₄, 500 ng mega primer, 50 ng template plasmid (GCC M5; pTE3101), and 2.5 U KOD Hot Start DNA polymerase (Novagen). The MEGAWHOP product was purified, digested with DpnI, and transformed into ElectroMAX DH5a (Thermo Fisher Scientific) to ensure a high number of transformants in the resulting libraries. To estimate the mutation rate for the different concentrations of MnCl₂ used in the error-prone PCR, the plasmids of ten randomly picked clones after MEGAWHOP were purified, sequenced and analyzed for nucleotide exchanges.

Protein Expression & Purification (Incl. SDS PAGE)

For the overexpression of GCC M5 and its mutant variants, the corresponding plasmid was transformed into chemically competent E. coli BL21-birA cells. Cells were grown on lysogeny broth (Miller) agar plates containing 100 μg/mL ampicillin and 50 μg/mL spectinomycin at 25° C. overnight. 8 L Golden lysogeny broth (Miller) containing 5 g/L yeast extract, 10 g/L tryptone, 10 g/L NaCl, 17 mM KH₂PO₄, 72 mM K₂HPO₄, and 0.4% glycerol was inoculated from the agar plate and incubated at 37° C. and 140 rpm. At OD₆₀₀=0.4-0.6 protein expression was induced with 500 μM IPTG and cells were incubated over night at 25° C. Cell harvesting at 8,000 g and 4° C. for 12 min and lysis by French pressing was followed by His-Trap purification using an Akta Start (GE Healthcare) linked to a HisTrap FF column (GE Healthcare). The purification buffer contained 50 mM HEPES pH 7.8 and 500 mM KCl, the elution was done with 500 mM imidazole. Protein desalting occurred via gel filtration chromatography using a HiLoad 16/600 Superdex 200 pg column (GE Healthcare) and a buffer containing 50 mM HEPES pH 7.8. and 150 mM KCl. Protein quantification occurred by absorbance measurement at 280 nm. Protein purity was validated by SDS-PAGE using 15 μg of purified protein on a 4-20% Mini-Protean TGX Precast Protein Gel (Biorad).

### Enzyme Assays

Enzyme activity assays were done in three different ways. Screening of randomly mutagenized GCC in order to produce a dataset to train an AI algorithm occurred via lysate-based measurements in plate readers. Prescreening of mutant variants that were predicted by the AI algorithm and selected by homology modelling and structural analysis was done with the same assay. The determination of carboxylation rates and ATP per carboxylation ratios occurred via spectrophotometric measurements with purified enzymes.

### Lysate-Based Measurements of Carboxylation Rate and ATP-Hydrolysis

GCC-encoding constructs or random-mutagenesis libraries of GCC were transformed into E. coli BL21_birA (see above) and eight colonies per construct were picked into 96-deep-well plates (PlateOne) with lysogeny broth (Miller) containing 100 μg/mL ampicillin and 50 μg/mL streptomycin. The plates were incubated over night at 37° C. with subsequent transfer into fresh 96-deep-well plates with lysogeny broth (Miller), 100 μg/mL ampicillin, 50 μg/mL spectinomycin, and 2 μg/mL biotin to an OD₆₀₀ of 0.1. Protein expression was induced with 0.25 mM isopropyl β-d-1-thiogalactopyranoside at an OD₆₀₀ of 0.4-0.6 and the cells were incubated over night at 25° C. The cells were lysed using CelLytic B (Sigma-Aldrich) and stored in 20% glycerol at −80° C. The enzyme activity was measured in a plate reader by the coupled enzyme assay with purified Malonyl-CoA Reductase from Chloroflexus aurantiacus (later referred to as CaMCR) as described earlier (Scheffen, loc. cit.). We used small-volume 384-well plates (Greiner Bio-One) with 2 μL cell extract, 100 mM 3-(N-morpholino)propanesulfonic acid (MOPS) pH 7.8, 1 mM ATP, 50 mM KHCO₃, 500 μg/mL CaMCR, 1 mM NADPH, 10 mM MgCl₂, and 1 mM glycolyl-CoA in a reaction volume of 10 μL. The absorbance of NADPH was measured at 340 nm and 37° C. for 5 h with intervals of 47 s in a plate reader (Tecan Infinite M Plex).

### Spectrophotometric Measurements of Carboxylation Rate

To measure the carboxylation rate of GCC, a coupled enzyme assay with CaMCR was performed. 100 mM MOPS pH 7.8, 50 mM KHCO₃, 2 mM ATP, 0.3 mM NADPH, 5 mM MgCl₂, 1.8 mg/mL CaMCR from Chloroflexus aurantiacus, and 0.01-1 mg/mL GCC were mixed in a cuvette and incubated for 2 min at 37° C. The reaction was started with 0.5 mM glycolyl-CoA and absorption was measured over time at λ=340 nm.

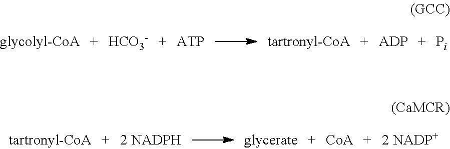

### Spectrophotometric Measurements of ATP Hydrolysis

To measure the ratio between ATP consumption and carboxylation of GCC, a coupled enzyme assay with CaMCR under ATP-limited conditions was performed. 100 mM MOPS pH 7.8, 50 mM KHCO₃, 0.15 mM ATP, 0.5 mM NADPH, 5 mM MgCl₂, 1.8 mg/mL CaMCR from Chloroflexus aurantiacus, and 0.05-3 mg/mL GCC were mixed in a cuvette and incubated for 2 min at 37° C. The reaction was started with 0.5 mM glycolyl-CoA and absorption was measured over time at λ=340 nm. The ATP per carboxylation ratio was calculated from the ratio between the ATP amount in the reaction mixture and the consumed amount of NADPH that is reflected by the absorbance drop during the reaction.

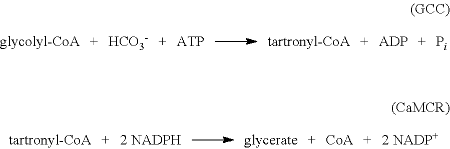

### AI-Predictions

Initially, 5,000 mutant variants covering approximately 35% of all possible single mutations were screened but no significant improvements of either the carboxylation rate or the ATP/CO₂-ratio were detected.

In order to develop GCC variants with improved kinetic properties an artificial intelligence (AI) algorithm was used. The prediction of beneficial mutations by AI algorithms was done with the company Exazyme (Berlin, Germany). A learning dataset to train the AI model was produced by generating a random mutagenesis library of pTE3101 (GCC M5), transforming it into chemically competent E. coli BL21-birA and colony picking into 96-deep-well plates (PlateOne) with lysogeny broth (Miller) containing 100 μg/mL ampicillin and 50 μg/mL spectinomycin. Expression, lysis and screening of GCC samples was done as described earlier (Scheffen, loc. cit.). 2,100 mutant variants were screened and from the obtained data a subset of representative candidates were selected for sequencing. 161 Samples were used to train the machine-learning model from Exazyme towards the prediction of beneficial mutations in GCC M5. The resulting list of all possible single mutations ranked by their efficiency was used as a template for identifying suitable candidates for biochemical characterization by homology model creation and structural investigation of promising mutations.

### Structural Modelling and Analysis

In order to assess mutations that were predicted by the AI algorithm, homology modelling of each promising mutant variant was performed using SWISS-MODEL. As a template for homology modelling of GCC mutations, the structure of the engineered GCC M5 from Methylobacterium extorquens (PDB ID 6YBQ) was used. Structural analysis of the models was done using PyMOL (the PyMOL Molecular Graphics System; version 1.8; Schrödinger). Modelling of glycolyl-CoA into the active site of GCC, respectively, was based on the positions of CoA in the GCC M5 structure and methylmalonyl-CoA in the structure of a methylmalonyl-CoA carboxytransferase from Propionibacterium freudenreichii (PDB ID 1ON3; 52% amino acid identity). Manual fitting and adjustments of the CoA thioesters reflecting differences in active-site architectures were done with COOT and PyMOL.

### Site-Directed Mutagenesis

Site-directed mutagenesis was used to construct mutant variants of GCC that were earlier predicted by the AI algorithm and selected by homology modelling and structural analysis. The introduction of novel mutations was done by single mutagenic oligonucleotide PCR as described elsewhere (Shenoy, Anal. Biochem. 319, 335-336, 2003). A 25 μL reaction mixture containing 0.5 μM primer, 3% (v/v) dimethyl sulfoxide, 50 ng template DNA (pTE3101) and Phusion High-Fidelity PCR Master Mix (NEB, M0531) was used for PCR and subsequently digested with DpnI (NEB, R0176) by adding 20 U to the reaction mixture and incubating 2 h at 37° C. 5 μL were transformed into chemically competent E. coli NEB Turbo cells and streaked out on lysogeny broth (Miller) agar plates with 50 μg/mL streptomycin. Three to six colonies were picked, cultivated in 10 mL lysogeny broth (Miller) with 50 μg/mL streptomycin for 12 h at 37° C. and 180 rpm and finally the plasmids isolated and sequenced to validate the mutagenesis.

### Site-Saturation Mutagenesis Library Generation:

Plasmid libraries of GCC M5 with residue 20 or 100 to be saturated with all amino acids were created by whole plasmid PCR with primer mixes containing different edited bases. Primers were designed with the 22c-trick to have reduced codon redundancy (Kille, et al., 2013, ACS Synthetic Biology 2 (2), 83-92). For the whole plasmid PCR, G20X primers (oDM0164, oDM0165, oDM0166 and oDM0170) or L100X primers (oDM0167, oDM0168, oDM0169 and oDM0171) were mixed in a 12:9:1:22 ratio to achieve equal amounts of each primer (Table 4), according to Kille et al. 2013 (Kille, et al., ACS synthetic biology, 2(2), 83-92). A 50 μL reaction mixture containing 0.5 μM primer mix, 3% (v/v) dimethyl sulfoxide, 100 ng template plasmid DNA encoding the GCC M5 (pTE3101) and Phusion High-Fidelity PCR Master Mix (NEB, M0531) was used for PCR and subsequently digested with DpnI (NEB, R0176) by adding 20 U to the reaction mixture and incubating 2 h at 37° C. After a PCR-clean up using the NucleoSpin Gel and PCR Clean-up Kit from Macherey-Nagel (REF 740609) and the corresponding protocol, 5 μL were transformed into chemically competent E. coli NEB Turbo cells and streaked out on lysogeny broth (Miller) agar plates with 50 μg/mL streptomycin. Colonies were flushed from the plate and plasmids were isolated. To ensure coverage of all plasmid variants in the libraries, at least 1300 colonies were collected, representing a 65-fold oversampling (meaning each of the proteinogenic amino acids is theoretically covered about 65-fold at both positions 20 or 100 of the GCC M5). Codon diversity was confirmed by sequencing of the library.

### CryoEM Sample Preparation and Data Collection

3 μL protein solution (1 mg/mL) in 50 mM HEPES pH 7.8. and 150 mM KCl containing 2 mM MgCl₂, 1 mM ATP, and 4 mM glycolyl-CoA were applied to QUANTIFOIL® R2/1 300 copper mesh grids that were glow-discharged for 90 s immediately before use and blotted for 3.5 s with blot force 4 at 100% humidity and 4° C. using a Vitrobot Mark IV (Thermo Scientific). Grids were plunge frozen in liquid ethane cooled by liquid nitrogen and used for data collection immediately.

CryoEM data were acquired on a Titan Krios G3i electron microscope (Thermo Scientific), operated at an acceleration voltage of 300 kV and equipped with a BioQuantum-K3 imaging filter (Gatan). Data were measured in electron counting mode at a nominal magnification of 105,000× (0.837 Å/pixel) with a total dose of 55 e⁻/Å² (55 fractions), using the aberration-free image-shift (AFIS) correction in EPU (Thermo Scientific). Five images were acquired per foil hole and the nominal defocus range for data collection was −0.5 to −2.0 μm.

### CryoEM Data Processing

Datasets were processed entirely in CryoSPARC (version 4.1 or 4.2; Punjani, et al., 2017, Nat Methods 14, 290-296). For all datasets dose-fractionated movies were gain-normalized, aligned, and dose-weighted using Patch Motion correction. The contrast transfer function (CTF) was determined using the Patch CTF routine. Information regarding cryoEM data collection, model refinement, and statistics are listed in Table 8.

### Processing of GCC M5 G20R

Blob picker and manual inspection of particles were used to extract an initial 837,101 particles with a box size of 256 pixels, which were used to build 2D classes. 2D classes with protein-like features were used to initialize template picking. After inspection and extraction with a box size of 256 pixels, this yielded 3,439,715 particles, which were used to build 2D classes. A total of 1,845,969 candidate particles were selected from 2D classes and used for ab-initio reconstruction and classification into 4 classes. Particles of the best-aligning class (647,870 particles) were subjected to non-uniform with per-particle defocus optimization, per-group CTF parameter optimization, and EWS correction. This yielded a map with 2.08 Å global resolution and a temperature factor of 66.1 Å², which was subsequently locally refined to yield a map with 2.03 Å global resolution and a temperature factor of 58.9 Å². The resulting map was B-factor sharpened by −40 Å². Further classification did not yield improved resolution.

### Processing of GCC M5 S100N

Blob picker and manual inspection of particles were used to extract an initial 620,147 particles with a box size of 500 pixels, which were used to build 50 2D classes. 2D classes with protein-like features were used to initialize template picking. After inspection and extraction with a box size of 500 pixels, this yielded 2,511,911 particles, which were used to build 2D classes. A total of 324,129 candidate particles were selected and used for ab-initio reconstruction and classification into 5 classes. Particles of the best-aligning class (113,824 particles) were subjected to non-uniform refinement with per-particle defocus optimization, per-group CTF parameter optimization, and EWS correction. This yielded a map with 2.36 Å global resolution and a temperature factor of 66.9 Å², which was subsequently locally refined to yield a map with 2.31 Å global resolution and a temperature factor of 60.6 Å². The resulting map was B-factor sharpened by −50 Å². Further classification did not yield improved resolution.

### Model Building and Refinement

CryoEM map fitting was initially performed in UCSF-ChimeraX (v1.6; Pettersen, et al., 2020, Protein Science, 30 1, 70-82) using GCC M5 (PDB 6YBQ) as template. The resulting model was manually built further in Coot (v0.9.8.3; Emsley, et al., 2010, Structural Biology, 66 4, 486-501). Automatic refinement of the structure was performed using phenix.real_space_refine of the Phenix (v1.20.1) software suite (Liebschner, et al., 2019, Structural Biology, 75 10, 861-877). Manual refinements and water picking were performed in Coot. The model statistics are listed in Table 8.

### Mass Photometry

Mass photometry (MP) measurements were carried out on microscope coverslips (1.5 H, 24×50 mm, Carl Roth) with CultureWell™ Reusable Gaskets (CW-50R-1.0, 50-3 mm diameter×1 mm depth) that had been washed by three consecutive rinses of water and isopropanol, prior to drying under a stream of pressurized air. Gaskets were assembled on microscope coverslips and placed on the stage of a TwoMP mass photometer (MP, Refeyn Ltd, Oxford, UK) with immersion oil. Measurements were carried out in 1× phosphate-buffered saline (PBS, 10 mM Na₂HPO₄, 1.8 mM KH₂PO₄, 137 mM NaCl, 2.7 mM KCl (pH 7.4)). To this end, 18 μL 1×PBS was used to focus the MP before 2 μL sample (1 μM protein) was added, rapidly mixed, and measured. Shortly before measuring, samples were prepared by diluting purified protein to 1 μM monomer concentration in buffer (50 mM HEPES, pH 7.8, 150 mM KCl), as determined by absorption at 280 nm. Data was acquired for 60 s at 100 frames per second using AcquireMP (Refeyn Ltd, Oxford, UK). MP contrast was calibrated to molecular masses using 50 nM of in-house purified protein mixture containing citrate-synthase complexes of known molecular masses ranging from 86 to 430 kDa. MP datasets were processed and analyzed using DiscoverMP (Refeyn Ltd, Oxford, UK). Details of MP image analysis have been described previously (Sonn-Segev, et al., 2020, Nat Commun 11, 1772).

### Flux Balance Analysis

To compare different versions of the tartronyl-CoA pathway to native plant photorespiration (as occurring during oxygenic photosynthesis), we performed stoichiometric modeling by applying flux balance analysis (FBA) with COBRApy (v0.20.0) (Ebrahim, et al., 2013, BMC systems biology, 7, 74). We used the same framework that had been used previously to compare the tartronyl-CoA (TaCo) pathway (comprising the GCC M5) with other routes (Scheffen, loc. cit.) and extended it with an additional TaCo pathway variant comprising the herein provided GCC M5 S100N. The employed stoichiometric model primarily considers the consumption of ATP, and reducing equivalents for each pathway. This is a widely employed standard method in the field of computational analysis of metabolic pathways and provides an indication on the energetic efficiency of the assessed pathways (Wilbert, et al., 2012, Metabolic Engineering, 14 3, 270-280; Berkvens, et al., Essays Biochem 30 Apr. 2024, 68 1, 41-51). Accordingly, the present analysis focuses on whether the herein provided GCC M5 S100N (which comprises a surprisingly low ATP/CO₂ consumption, as is shown, inter alia, in Table 6), would also be advantageous over the GCC M5 in the context of the full TaCo pathway. Accordingly, for each pathway, we calculated the consumption of ATP, NAD(P)H and reduced ferredoxins, as well as the required turns of the CBB cycle (including RuBisCO) to produce one unit of 3-phosphoglycerate (3PG). For this purpose, we constructed a simplified metabolic model consisting of the Calvin-Benson-Bassham (CBB) cycle, specific reactions of each considered photorespiratory pathway, and generic/artificial cofactor regeneration and interconversion reactions (e.g., ADP+inorganic phosphate→ATP; NAD+→NADH and NADH+NADP+→NAD++NADPH; ATP+AMP→2 ADP; oxidized ferredoxin→reduced ferredoxin). We note that transfer of electrons from NADH to NADPH, and vice-versa, is freely possible in this model without any ATP-investment via a generic transhydrogenase reaction (simulating photosynthetic production of NADPH). We assumed a RuBisCO carboxylation-to-oxygenation ratio of 3:1 (i.e. 25% of all RuBisCO reactions being oxygenations) (Walker, loc.cit.; Fu, loc. cit.; Sharkey, 1988, Physiologia Pantarum, 73 1; 147-152). To compare the yield of all pathways, we calculated their total required “ATP-equivalents” to produce 3-phosphoglycerate (3PG) from CO₂, by using the conversions 1 NAD(P)H=2.5 ATP (Ferguson, 1986, Trends in Biochemical Sciences, 119, 351-353; Hinkle, 2005, Biochimica et Biophysica Acta (BBA)—Bioenergetics, 1706 1-2, 1-11) and 2 reduced ferredoxins=1 NAD(P)H.

### Results

The enzyme GCC M5 was developed for a synthetic CO₂ fixation pathway, which is capable to catalyze the carboxylation of glycolyl-CoA to tartronyl-CoA (Scheffen, loc. cit.).

However, GCC M5 has an ATP per carboxylation ratio of around 4 although the theoretical ratio is 1. Thus, further enzyme engineering was done to provide candidates with lowered ATP consumption and improved energetic efficiency.

As described earlier, the application of random mutagenesis, rational design, directed evolution, coupled with high-throughput screening of mutant variants of GCC M3 produced the improved variants GCC M4 and GCC M5.

In order to continue this approach in a new iteration, a random mutagenesis library of GCC M5 was constructed where pccB_M5, the carboxyl transferase encoding gene of the carboxylase was targeted by error-prone PCR. Large-scale screening of the library was done as described earlier (Scheffen, loc. cit.). 5,000 mutant variants covering approximately 35% of all possible single mutations were screened but no significant improvements of either the carboxylation rate or the ATP/CO₂-ratio were detected. Thus, conventional methods to identify variants with improved kinetic properties failed to provide results.

Further approaches were tried to develop improved GCCs (based on M5), which however did not yield improvements. For example, we tried (i) searching for homologous enzymes that naturally bear some of the M5 mutations, (ii) the usage of molecular dynamics simulations to mimic the reaction mechanism and predict new mutations, (iii) the development of an in vivo selection system for GCC and (iv) repeating the directed evolution workflow that was already used before to produce M5 (Scheffen, loc. cit.).

Despite these failed attempts, it was decided to subsequently use the generated screening data to train an artificial intelligence-(AI) model towards the prediction of beneficial mutations in GCC M5, i.e. without the screening data generated in our lab the AI model could not have been developed and the AI could not have produced any useful results.

The resulting list of all possible single mutations (approx. 10,000 mutations) was ranked by the AI model by their efficiency. However, we decided not to simply proceed with biochemical characterization of the top hits. Rather, we further investigated and assessed the proposed list of mutations in order to identify candidates for biochemical characterization which might in our view indeed show promise for improved kinetic properties. From the top 1% (covering 105 predictions) homology models based on the pdb structure 6YBQ were constructed and structural investigation of the mutant variants was done.

The mutation at position 20 was selected although it is not present in or near the active center and we would therefore not have expected it to have an impact on enzymatic activity. However, mutations at position 20 were present several times in the top 1% predictions, which indicated to us that the position might be relevant. Other mutations were selected, which we assumed might have an effect on enzymatic activity and/or which had a high frequency in the top 1% predictions (the latter was the case for mutations at positions 53 and 510). Based on the above considerations, we selected seven mutant variants ranked in the top 1% to be tested in vitro (Table 5).

Additionally, we selected three mutant variants that were ranked in the top 5%. Mutations at these positions had been tested earlier; however, some (like S100N) had failed to show improved kinetic properties while other amino acid substitutions at positions 502 and 407 had been tested earlier (see Scheffen, loc. cit.). Despite these earlier failures we decided to test these selected three variants, too. None of the earlier variants was known to have lowered ATP consumption.

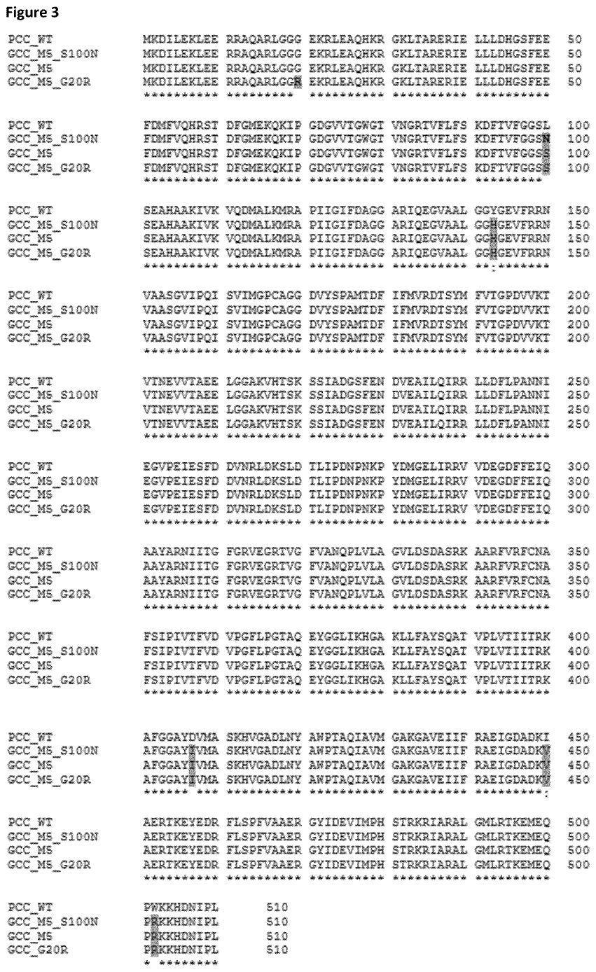

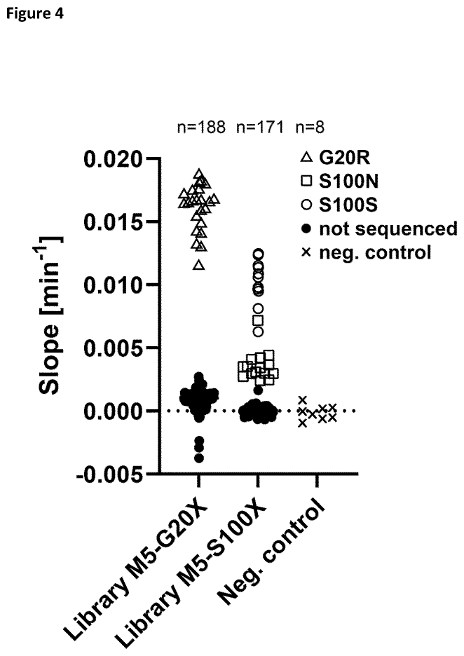

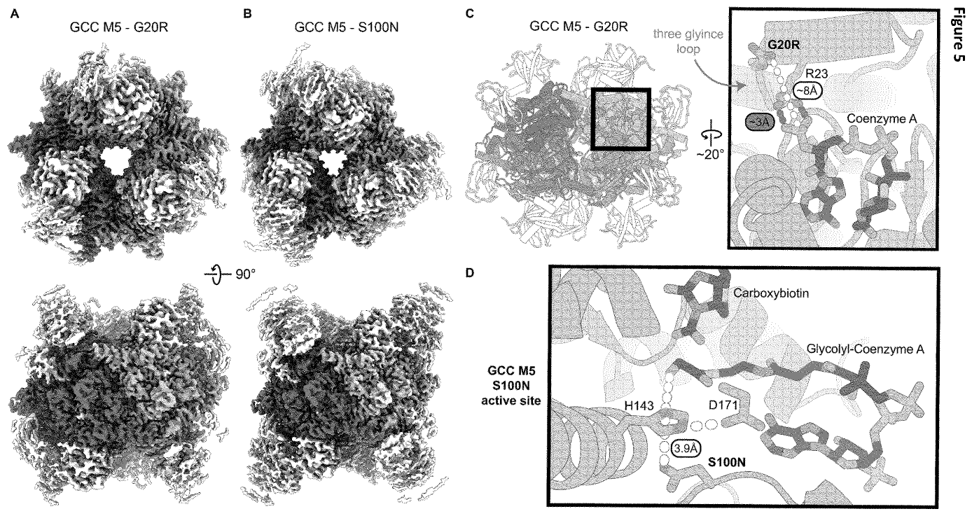

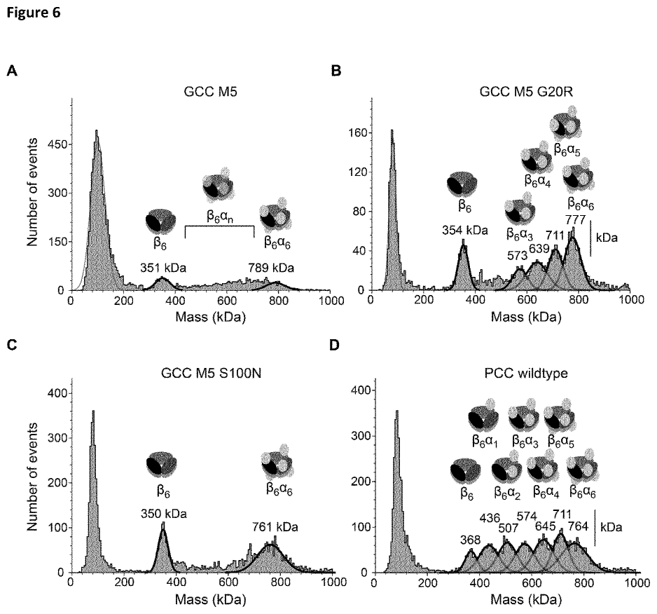

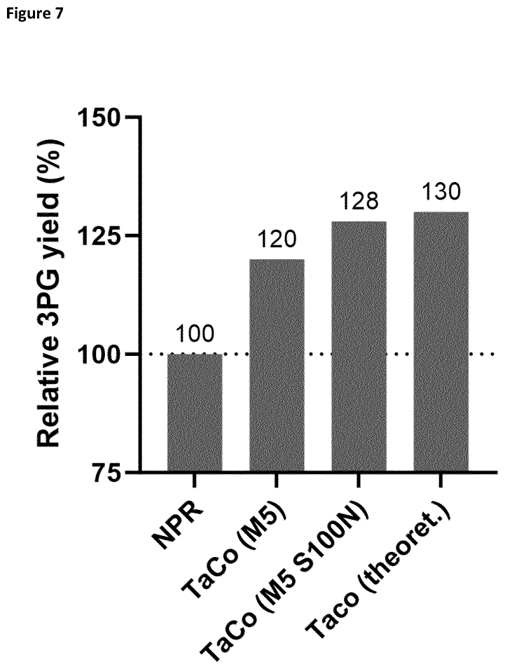

The analyzed mutations are shown in Table 5 below and are partly additionally illustrated by a sequence alignment in FIG. 3.

TABLE 5

| Candidate¹ | | | Candidate² |
|---|---|---|---|
| (mutations | | | (mutations |
| versus GCC | | | versus |
| M5) | Rank | Top ratio | PCC) |
| G20R | 3/10019 | 0.03% | G20R, L100S, Y143H, |
| (SEQ ID NO: 3) | | | D407I, I450V, |
| | | | W502R |
| L8F | 5/10019 | 0.05% | L8F, L100S, Y143H, |
| (SEQ ID NO: 2) | | | D407I, I450V, |
| | | | W502R |
| M53Q | 20/10019 | 0.20% | M53Q, L100S, |
| (SEQ ID NO: 5) | | | Y143H, D407I, |
| | | | I450V, W502R |
| M53E | 26/10019 | 0.26% | M53E, L100S, |
| (SEQ ID NO: 4) | | | Y143H, D407I, |
| | | | I450V, W502R |
| L510F | 46/10019 | 0.46% | L100S, Y143H, |
| (SEQ ID NO: | | | D407I, I450V, |
| 11) | | | W502R, L510F |
| T495N | 102/10019 | 1.02% | L100S, Y143H, |
| (SEQ ID NO: 9) | | | D407I, I450V, |
| | | | T495N, W502R |
| M64R | 104/10019 | 1.04% | M64R, L100S, |
| (SEQ ID NO: 6) | | | Y143H, D407I, |
| | | | I450V, W502R |
| S100N | 303/10019 | 3.02% | L100N, Y143H, |
| (SEQ ID NO: 7) | | | D407I, I450V, |
| | | | W502R |
| R502H | 382/10019 | 3.81% | L100S, Y143H, |
| (SEQ ID NO: | | | D407I, I450V, |
| 10) | | | W502H |
| I407V | 410/10019 | 4.09% | L100S, Y143H, |
| (SEQ ID NO: 8) | | | D407V, I450V, |
| | | | W502R |

¹Mutations of the variant candidates in the first column from the left are indicated versus the GCC M5 variant (SEQ ID NO. 1).
²Mutations of the respective same variant candidates in the last column are indicated versus the wild-type Propionyl-CoA Carboxylase (SEQ ID NO: 12).

In photometric measurements of glycolyl-CoA carboxylation activity with cell lysates, all mutant variants except for M64R showed significant activities and were purified for further analytics. The lack of activity of M64R demonstrates that the AI model can only provide a pre-selection of potentially beneficial positions for amino acid substitutions but cannot reliably predict whether a mutant variant in fact has activity, let alone whether the activity is indeed improved. Further, the positions from the pre-selection list generated by the AI for biochemical characterization needed to be selected following considerations which could not be modelled by AI.

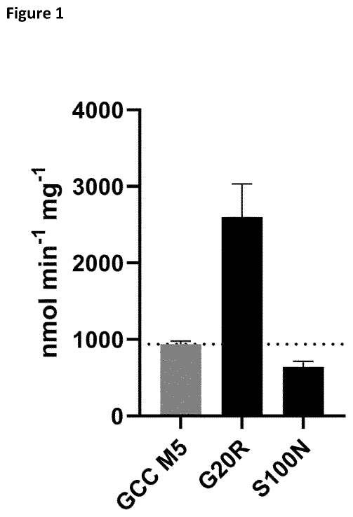

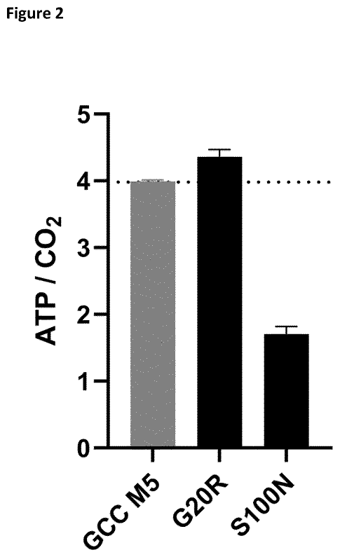

Specific activities for glycolyl-CoA carboxylation as well as ATP/CO₂-ratios were measured spectrophotometrically (Table 6). The data show that we developed two evolved variants of GCC M5. Candidate G20R shows a 2-3 fold increase in the carboxylation activity while candidate L100N has a reduced ATP consumption of more than 50% (FIG. 1 and FIG. 2, respectively). Structural analysis of these mutant variants by cryo-electron microscopy will be used to investigate the role of these residues in the overall complex and how they affect catalysis. Additionally, site-directed mutagenesis with will be used to construct the mutant variants G20A, G20H, G20K and G20Y that are expected to have an impact on catalysis. Saturation mutagenesis on positions 20 and 100 will also be done to cover all possible mutations at these positions and get further insights into the role of these positions and the required residue properties to improve glycolyl-CoA carboxylation.

TABLE 6

| Candidate³ | Specific activity (nmol min⁻¹ mg⁻¹) | ATP/CO₂-ratio |
|---|---|---|
| GCC M5 | 937 ± 40 | 4.00 ± 0.02 |
| (reference) | | |
| L8F | 677 ± 51 | 4.40 ± 0.06 |
| G20R | 2602 ± 430 | 4.36 ± 0.11 |
| M53E | n.d. | n.m. |
| M53Q | n.d. | n.m. |
| S100N | 642 ± 73 | 1.71 ± 0.11 |
| I407V | 640 ± 81 | 6.65 ± 0.08 |
| T495N | 413 ± 72 | 4.45 ± 0.34 |
| R502H | 630 ± 55 | 4.96 ± 0.21 |
| L510F | n.d. | n.m. |

n.d. = not detectable,
n.m. = not measured

The results show that most of the AI predicted mutant variants even among the top 1% did not show improved kinetic properties and even if the position had a high frequency in the top 1% mutations (e.g. position 53 or 510). Mutations at positions 53 and 510 even showed no detectable activity. Other predicted mutant variants showed activity, but to a lesser extent than reference GCC M5. Also, mutations at positions that had earlier been mutated (like position 407 and 502) did not provide improved kinetic properties. We were therefore surprised that mutations at positions 20 and 100 showed improved activity/kinetic properties.

We then performed more in detail biochemical characterizations determining Vₘₐₓ, k_{cat} and K_{M} values for this enzyme variant using LC-MS assays, which underscored the catalytic 3 Mutations of the variant candidates are indicated versus the GCC M5 variant (SEQ ID NO. improvement over GCC M5 (Table 7). While the apparent K_{M} value for glycolyl-CoA of the G20R variant slightly increased, the Vₘₐₓ was 1.8-fold higher than GCC M5 and thus the G20R variant with a k_{cat} of 9.8±0.2 has high prospects for GCC-based applications such as, for example, its use for the tartronyl-CoA pathway with higher production rates (Scheffen loc. cit.). These results further confirm the surprisingly high and advantageous enzymatic activity of the G20R variant detailed for example in Table 6, particularly over GCC M5. In this variant, the substitution of Gly by Arg at position 20 on a surface loop of the 3-core strongly increased the carboxylation rate, whereas the ATP per carboxylation ratio only changed marginally (FIGS. 1 and 2). As detailed, for example in Table 6, the S100N variant showed a significantly decreased ATP to carboxylation ratio of 1.7±0.1 ATP, lowering the energy demand for the reaction by 60% compared to GCC M5 (4.0±0.0 ATP per glycolyl-CoA carboxylation). To evaluate whether other mutations at positions 20 and 100 showed beneficial impacts on GCC's catalytical properties, we tested site-saturation libraries for both positions and applied the lysate-based screen (FIG. 4). However, we could not detect any other substitutions with activity apart from G20R and S100N or the S100S (i.e., GCC M5) variants. These findings strongly support that it could not have been foreseen that substitutions at positions 20 and/or 100 of the GCC M5 (in particular with Arg and Asn, respectively) result in enzymatically active GCC variants (much less that such substitutions would result in GCC variants with even improved characteristics, particularly over GCC M5, such as enzymatic activity and ATP/CO₂ ratio).

To identify structural changes that might be responsible for the catalytic improvements of the G20R or S100N variants, we solved their cryo-EM structures at 2.05 and 2.31 Å, respectively (FIGS. 5A&B). The G20R substitution is located ~8 Å away from the 3′-phosphate of coenzyme A and surprisingly does not appear to interact with other residues or the substrate (FIG. 5C). The lack of defined contact points in the cryo-EM structure is reflected in only weak electron density observed for the side chain of Arg20, indicating a high degree of side chain flexibility. Arg20 is positioned in a flexible loop, where it is preceded by two glycine residues, which add to its increased flexibility. Based on G20R's position on the top rim of the β₆ core of GCC, we suspected that it might help stabilize the interaction with the α-subunit and indirectly also facilitate CoA positioning (FIG. 5C). Indeed, in mass photometry (MP) measurements, the G20R variant formed more higher mass complexes relative to GCC M5, indicating a more stable complex formation (FIGS. 6A&B). It has recently been shown that complex formation of propionyl-CoA carboxylases occurs is a dynamic process with an equilibrium between assembly and disassembly of complexes and β₆α₆ being the most active complex form (Lee, et al., 2023, J Struct Biol X, 7, 100088). Thus, the higher fraction of α-subunits bound to the β₆ core likely explains the higher in vitro activity of the G20R mutant. Thus, it appears conceivable that employing the herein provided GCC M5 G20R (or variants thereof) in metabolic pathways like the tartronyl-CoA pathway results in higher product formation or reduced metabolic burden in living organisms (as compared to the same metabolic pathway comprising the GCC M5 instead of the GCC M5 G20R variant) due to the lower quantities of enzyme that is needed to reach a comparable reaction rate.

The S100N substitution is located in the periphery of the active site, at a position that had previously been targeted during engineering efforts of GCC M5, in which this position was engineered to be serine. The Asn100 substitution is in close proximity to His143, which was proposed to coordinate the hydroxyl group of glycolyl-CoA (FIG. 5D). While active site overlays of the S100N variant and GCC M5 look almost identical, we assume that the Asn100 forces His143 into a more favorable rotamer conformation for substrate binding and catalysis, enabling carboxylation to occur more efficiently. This is supported by the fact that His143 is categorized as a rotamer outlier in all subunits of the S100N cryo-EM structure, which is not the case for the GCC M5 or G20R variant structures. Such minor movements of the His143 side chain towards glycolyl-CoA might facilitate improved substrate orientation/positioning and thus reduce the unfruitful decarboxylation of carboxybiotin (i.e., the release of CO₂ from carboxybiotin without a transfer onto the substrate). This in turn decreases the reaction's energy requirement in ATP. While the S100N variant exhibits a slightly increased proportion of higher mass oligomeric complexes in MP experiments (FIG. 6C), the complex distribution remained similar to that of GCC M5 and PCC (FIGS. 6A&D). All investigated variants, including the PCC wildtype (FIG. 6D), formed a stable β₆ core with variable amounts of α-subunits bound in MP measurements. Nonetheless, as is mentioned herein above, the complex formation of the herein disclosed GCC enzymes is a dynamic process (which means that the subunits may dissociate). Thus, it might be of interest to further improve the stability of such complexes (in particular for efficient purification thereof and/or in vitro applications). In order to further improve the formation of active GCC complexes with fully bound α-subunits (β6α6), one might, for example, increase the expression of at-subunits compared to β-subunits to saturate the β-core.

TABLE 7
Kinetic properties of GCC M5 variants G20R and S100N.

| | | Vₘₐₓ | | | | |
|---|---|---|---|---|---|---|
| | | [nmol min⁻¹ | k_{cat} | App. K_{M} | k_{cat}/K_{M} | |
| Enzyme | Substrate | mg⁻¹] | [s⁻¹] | [mM] | [s⁻¹ M⁻¹] | Reference |
| GCC M5 | Glycolyl-CoA | 2590 ± 130 | 5.6 ± 0.3 | 0.15 ± 0.03 | 3.63 × 10⁴ | Scheffen |
| | | | | | | loc. cit. |
| G20R | Glycolyl-CoA | 4520 ± 90 | 9.8 ± 0.2 | 0.27 ± 0.02 | 3.64 × 10⁴ | This work |
| | Acetyl-CoA | 2250 ± 200 | 4.9 ± 0.4 | 0.38 ± 0.10 | 1.30 × 10⁴ | This work |
| S100N | Glycolyl-CoA | 790 ± 40 | 1.7 ± 0.1 | 0.32 ± 0.05 | 5.36 × 10³ | This work |
| | Acetyl-CoA | 174 ± 12 | 0.38 ± 0.03 | 0.63 ± 0.10 | 5.96 × 10² | This work |

The data represent means±s.d., as determined from n=18 independent measurements using nonlinear regression.

TABLE 8
CryoEM data collection, refinement, and model statistics.
Data collection

| | | |
|---|---|---|
| Model | GCC M5 G20R | GCC M5 S100N |
| Accessions | PDB 8PN7, EMD-17777 | PDB 8PN8, EMD-17778 |
| Microscope | Titan Krios G3i | Titan Krios G3i |
| Voltage (kV) | 300 | 300 |
| Camera | K3 | K3 |
| Magnification | 105,000 | 105,000 |
| Pixel size at detector | 0.837 | 0.837 |
| (Å/pixel) | | |
| Total electron exposure | 55 | 55 |
| (e⁻/Å²) | | |
| Exposure rate | 15 | 15 |
| (e⁻/pixel/sec) | | |
| Frames per exposure | 55 | 55 |
| Defocus range (μm) | −0.5 to −2.0 | −0.5 to −2.0 |
| Automation software | EPU, CryoSPARC | EPU, CryoSPARC |
| Energy filter slit width | −30 eV | −30 eV |
| Micrographs collected | 9713 | 9783 |
| (no.) | | |
| Micrographs used (no.) | 9713 | 9783 |
| Total extracted particles | 3,439,715 | 2,511,911 |
| (no.) | | |
| Refined particles (no.) | 647,870 | 113,824 |
| Final particles (no.) | 647,870 | 113,824 |
| Point-group | D3 | D3 |
| Resolution (global, Å) | 2.03 | 2.31 |
| FSC_{0.143} | 2.60/2.03 | 3.2/2.31 |
| | (unmasked/masked) | (unmasked/masked) |
| Resolution range (local, Å) | 1.85-10.31 | 1.83-28.83 |
| Map sharpening β-factor | −40.0 | −50.0 |
| (Å²) | | |
| Map sharpening methods | CryoSPARC sharpening | CryoSPARC sharpening |

Model Refinement

| | | |
|---|---|---|
| Refinement package | Phenix | Phenix |
| method | real space | real space |
| resolution cutoff | 2.05 | 2.36 |
| Protein residues | 4308 | 4314 |
| Ligands | 6× CoA, 6× biotin | 6× CoA, 6× biotin |

Model-Map scores

| | | |
|---|---|---|
| CC_{Map} | 0.76 | 0.78 |
| avg. FSC_{0.143} | 2.05/1.97 | 2.35/2.08 |
| | (unmasked/masked) | (unmasked/masked) |

β-factors (Å²) (min/max/mean)

| | | |
|---|---|---|
| Protein residues | 1.95/140.39/54.63 | 4.25/173.70/62.54 |
| Ligands | 71.73/101.63/84.38 | 78.91/115.73/95.36 |
| Waters | 22.85/80.84/52.66 | 29.22/93.26/51.68 |
| R.M.S. Bond lengths (Å) | 0.002 | 0.002 |
| R.M.S. Bond angles (°) | 0.464 | 0.432 |

Validation

| | | |
|---|---|---|
| MolProbity score | 1.22 | 1.22 |
| CaBLAM outliers | 1.90 | 1.71 |
| Clashscore | 4.47 | 4.39 |
| Poor rotamers (%) | 0.35 | 0.43 |
| C-beta deviations | 0.00 | 0.00 |
| EMRinger score | 5.80 | 5.17 |
| Ramachandran favored | 98.53 | 98.97 |
| (%) | | |
| Ramachandran outliers | 0.00 | 0.00 |
| (%) | | |

Having demonstrated the improvements in carboxylation rate and ATP consumption of GCC M5 G20R and S100N in vitro, we finally analyzed its potential in improving natural CO₂ fixation in plants via the tartronyl-CoA pathway (Scheffen, loc. cit.). Using flux balance analysis (Orth, et al, 2010, Nat Biotechnol 28, 245-248), we evaluated the energetic requirements of natural plant photorespiration and different versions of the tartronyl-CoA (TaCo) pathway (Scheffen, loc. cit.) in combination with the Calvin-Benson-Bassham (CBB) cycle of photosynthesis (FIG. 7, Table 9). The model considered ATP and NADPH requirements and predicted the theoretical yield of 3PG, the product of the CBB cycle that is produced from 3 molecules CO₂. Since GCC M5 G20R caused a kinetic improvement but the flux balance analysis (FBA) only considers stoichiometric data, we limited our analysis to tartronyl-CoA pathway versions containing either the prior GCC M5, the novel variant GCC M5 S100N, or a theoretical GCC without futile ATP hydrolysis. The model revealed an increased theoretical 3PG yield for all TaCo versions with an improvement of 20% for the TaCo pathway with GCC M5 and 28% improvement for the version with GCC S100N, which is very close to the theoretical maximum at 30% improvement (FIG. 7, Table 9). The tartronyl-CoA pathway converts photorespiration into a process that assimilates CO₂ rather than releasing it, thus doubling its carbon efficiency from 75% to 150% (Bar-Even, 2018, Plant Science, 273, 71-83; Scheffen, loc. cit.; Trudeau, loc. cit.; Marchal, et al, 2023, ACS Synth. Biol. 12 12, 3521-3530). In terms of energetic efficiency, coupling the CBB cycle to the TaCo pathway with GCC S100N requires 18% less ATP and 24% fewer reducing equivalents compared to natural plant photorespiration and 13% less ATP compared to TaCo with GCC M5 (FIG. 7, Table 9). Accordingly, the herein provided GCC M5 S100N variant (or GCC variants carrying the S100N substitution) was shown not only to comprise a surprising and advantageous ATP/CO₂ ratio (i.e., require less ATP per carboxylation reaction), but when employed in the context of the TaCo pathway, was shown in silico to result in approx. 28% more efficient carboxylation rates. Resultingly, a plant comprising the TaCo pathway (instead of or in addition to native plant photorespiration pathway) with the herein provided GCC M5 S100N should be 28% more energy efficient, which could result in up to approx. 28% higher biomass yields—an increase that is highly relevant to, for example, the agricultural sector, however, also various other sectors.

TABLE 9
Comparison of natural photorespiration and the TaCo
pathway, adapted from (Scheffen, loc. cit.).

| | | TaCo pathway | TaCo | TaCo |
|---|---|---|---|---|
| | | (theoretical) | pathway | pathway |
| | | (Scheffen, loc. | M5) | (M5 + S100N) |
| | | cit.; Trudeau, | ((Scheffen, | (as provided |
| | NPR | loc. cit.) | loc. cit.) | herein) |
| CO₂ release | yes | no | no | no |
| Place of CO₂ | mitochondria | — | — | — |
| release | | | | |
| Carbon | 75% | 150% | 150% | 150% |
| efficiency ^{a} | | | | |
| Route for | complete | Photorespiratory | Photorespiratory | Photorespiratory |
| (re-)fixation | CBB cycle | bypass fixes CO₂ | bypass fixes CO₂ | bypass fixes CO₂ |
| of released CO₂ | | | | |
| NH₃ release | yes | no | no | no |
| Required | 11 | 6 ^{b} | 6 ^{b} | 6 ^{b} |
| enzymes | | | | |
| (excluding CBB | | | | |
| cycle) | | | | |
| Required turns | 4.8 | 3 | 3 | 3 |
| of the CBB cycle | | | | |
| RuBisCO | 3.6 | 2.25 | 2.25 | 2.25 |
| carboxylations | | | | |
| (75%) | | | | |
| RuBisCO | 1.2 | 0.75 | 0.75 | 0.75 |
| oxygenations | | | | |
| (25%) | | | | |
| consumed ATP | 14 | 11 | 13.25 | 11.525 |
| consumed | 8 NAD(P)H + | 6.5 NAD(P)H | 6.5 NAD(P)H | 6.5 NAD(P)H |
| reducing | 0.6 Fd²⁻ | | | |
| equivalents | | | | |
| total consumed | 35.5 | 27.25 | 29.5 | 27.775 |
| “ATP- | | | | |
| equivalents” ^{c} | | | | |
| Energetic | 100% | 130% | 120% | 128% |
| efficiency | | | | |
| (relative to | | | | |
| NPR) | | | | |

^{a} for conversion of 2-phosphoglycolate into 3-phosphoglycerate
^{b} considering the two reactions of TCR (tartronyl-CoA reductase) as two separate enzymes
^{c} required “ATP-equivalents” were calculated based on the equations: 1 NAD(P)H = 2.5 ATP (Ferguson, 2010, Proceedings of the National Academy of Sciences of the United States of America, 107 39, 16755-16756; Hinkle loc. cit.), 2 reduced ferredoxins (1 Fd²⁻) = 1 NAD(P)H

bold numbers are the result of flux-balance analysis, calculated for net conversion of 3×CO₂ into one unit of 3-phosphoglycerate via the CBB cycle and the respective photorespiration route (see the methods section herein above for more details)

Abbreviations: Fd²⁻=2 reduced ferredoxins; NPR=native plant photorespiration (glycine decarboxylation route); TaCo: tartronyl-CoA pathway; M5=includes futile ATP-hydrolysis by the enzyme “GCC(M5)” in the TaCo pathway (4.0 ATP per carboxylation reaction formed); M5+S100N=includes futile ATP-hydrolysis by the optimized enzyme “GCC M5 S100N” in the TaCo pathway (1.7 ATP per carboxylation reaction formed).

All references cited herein are fully incorporated by reference. Having now fully described the invention, it will be understood by a person skilled in the art that the invention may be practiced within a wide and equivalent range of conditions, parameters and the like, without affecting the spirit or scope of the invention or any embodiment thereof.

1. A Glycolyl-CoA Carboxylase (GCC), wherein said GCC is characterized in that:
it comprises an amino acid sequence having at least 60% sequence identity to SEQ ID NO:1, and
it has one or more amino acid substitutions, deletions or insertions at a position selected from the group consisting of positions 20 and 100 in the amino acid sequence shown in SEQ ID NO: 1 or at a position corresponding to any of these positions,
preferably wherein the GCC has an improved activity over a reference GCC, preferably wherein the reference GCC comprises the amino acid sequence of SEQ ID NO:1.

2. The GCC of claim 1, wherein
(1) the amino acid at position 20 in the amino acid sequence shown in SEQ ID NO: 1 or at a position corresponding to this position, is substituted with arginine,
preferably wherein the amino acid that is substituted is glycine; and/or
(2) the amino acid at position 100 in the amino acid sequence shown in SEQ ID NO: 1 or at a position corresponding to this position, is substituted with asparagine,
preferably wherein the amino acid that is substituted is serine.

3. The GCC of claim 1 or 2, wherein the improved activity is improved energetic efficiency.

4. The GCC of claim 3, wherein the improved energetic efficiency is reduced ATP consumption, preferably reduced by at least 50%.

5. The GCC of any one of claims 1 to 4, wherein the GCC has an ATP/CO₂ ratio of lower than about 4.00±0.02.

6. The GCC of any one of claims 1 to 5, wherein the GCC has an ATP/CO₂ ratio of about 1.71±0.11.

7. The GCC of any one of claims 1 to 6, wherein the improved activity is increase in carboxylation activity, preferably an at least 2- or 3-fold increase.

8. The GCC of claim 7, wherein the increase in carboxylation activity is an increase in conversion of glycolyl-CoA into tartronyl-CoA.

9. The GCC of any one of claims 1 to 8, wherein the GCC has a catalytic rate of carboxylation of glycolyl-CoA (conversion of glycolyl-CoA into tartronyl-CoA) of higher than about 5.6±0.3 s⁻¹.

10. The GCC of any one of claims 1 to 9, wherein the specific activity of the GCC is higher than about 937±40 nmol glycolyl-CoA min⁻¹ mg⁻¹.

11. The GCC of claim 10, wherein the specific activity of the GCC is about 2602±430 nmol glycolyl-CoA min⁻¹ mg⁻¹.

12. A nucleic acid molecule encoding the GCC of any one of claims 1 to 11.

13. A vector comprising the nucleic acid molecule of claim 12.

14. An organelle comprising the nucleic acid molecule of claim 12 or the vector of claim 13.

15. A host cell comprising the nucleic acid molecule of claim 12, the vector of claim 13, or the organelle of claim 14.

16. A tissue comprising the host cell of claim 15.

17. An organism comprising the nucleic acid molecule of claim 12, the vector of claim 13 or the organelle of claim 14.

18. The organism of claim 17, wherein the organism is a plant, algae, or a microorganism.

19. The organism of claim 18, wherein the microorganism is a bacterium.

20. The organelle of claim 14, the host cell of claim 15, the tissue of claim 16, or the organism of any one of claims 17 to 19, wherein said organelle, said host cell, said tissue, or said organism has a higher conversion rate of glycolyl-CoA into tartronyl-CoA than the corresponding organelle, host cell, tissue, or organism comprising a reference GCC, preferably the reference GCC comprising SEQ ID NO: 1.

21. The organelle of claim 14, the host cell of claim 15, the tissue of claim 16, or the organism of any one of claims 17 to 19, wherein said organelle, said host cell, said tissue, or said organism has a higher growth rate and/or carbon yield than the corresponding organelle, host cell, tissue, or organism comprising a reference GCC, preferably the reference GCC comprising SEQ ID NO: 1.

22. Use of the GCC of any one of claims 1 to 11, the organelle of claim 14, the host cell of claim 15, the tissue of claim 16, or the organism of any one of claims 17 to 19 for CO₂-fixation and/or for a photosynthetic process.

23. Use of the GCC of any one of claims 1 to 11, the organelle of claim 14, the host cell of claim 15, the tissue of claim 16, or the organism of any one of claims 17 to 19 for decomposition of a synthetic material.

24. Use of claim 23, wherein the synthetic material is a polyester.

25. Use of claim 24, wherein the polyester is polyethylene terephthalate.

26. Use of the GCC of any one of claims 1 to 11, the organelle of claim 14, the host cell of claim 15, the tissue of claim 16, or the organism of any one of claims 17 to 19 for decomposition of ethylene glycol and/or glycolate.

27. A method for producing biomass using the GCC of any one of claims 1 to 11, the organelle of claim 14, the host cell of claim 15, the tissue of claim 16, or the organism of any one of claims 17 to 19.

28. A method for decomposition of a synthetic material using the GCC of any one of claims 1 to 11, the organelle of claim 14, the host cell of claim 15, the tissue of claim 16, or the organism of any one of claims 17 to 19.

29. The method of claim 28, wherein the synthetic material is a polyester.

30. The method of claim 29, wherein the polyester is polyethylene terephthalate.

31. A method for decomposition of ethylene glycol and/or glycolate using the GCC of any one of claims 1 to 11, the organelle of claim 14, the host cell of claim 15, the tissue of claim 16, or the organism of any one of claims 17 to 19.

32. A composition comprising the GCC of any one of claims 1 to 11, the nucleic acid of claim 12, the vector of claim 13, the organelle of claim 14, the host cell of claim 15, the tissue of claim 16, or the organism of any one of claims 17 to 19.


SEQUENCE LISTING (60 sequences)

SEQ ID NO: 1 - protein, 510 aa, synthetic construct
MKDILEKLEERRAQARLGGGEKRLEAQHKRGKLTARERIELLLDHGSFEEFDMFVQHRSTDFGMEKQKIPGDGVVTGWGTVNGRTVFLFSKDFTVFGGSSSEAHAAKIVKVQDMALKMRAPIIGIFDAGGARIQEGVAALGGHGEVFRRNVAASGVIPQISVIMGPCAGGDVYSPAMTDFIFMVRDTSYMFVTGPDVVKTVTNEVVTAEELGGAKVHTSKSSIADGSFENDVEAILQIRRLLDFLPANNIEGVPEIESFDDVNRLDKSLDTLIPDNPNKPYDMGELIRRVVDEGDFFEIQAAYARNIITGFGRVEGRTVGFVANQPLVLAGVLDSDASRKAARFVRFCNAFSIPIVTFVDVPGFLPGTAQEYGGLIKHGAKLLFAYSQATVPLVTIITRKAFGGAYIVMASKHVGADLNYAWPTAQIAVMGAKGAVEIIFRAEIGDADKVAERTKEYEDRFLSPFVAAERGYIDEVIMPHSTRKRIARALGMLRTKEMEQPRKKHDNIPL

SEQ ID NO: 2 - protein, 510 aa, synthetic construct
MKDILEKFEERRAQARLGGGEKRLEAQHKRGKLTARERIELLLDHGSFEEFDMFVQHRSTDFGMEKQKIPGDGVVTGWGTVNGRTVFLFSKDFTVFGGSSSEAHAAKIVKVQDMALKMRAPIIGIFDAGGARIQEGVAALGGHGEVFRRNVAASGVIPQISVIMGPCAGGDVYSPAMTDFIFMVRDTSYMFVTGPDVVKTVTNEVVTAEELGGAKVHTSKSSIADGSFENDVEAILQIRRLLDFLPANNIEGVPEIESFDDVNRLDKSLDTLIPDNPNKPYDMGELIRRVVDEGDFFEIQAAYARNIITGFGRVEGRTVGFVANQPLVLAGVLDSDASRKAARFVRFCNAFSIPIVTFVDVPGFLPGTAQEYGGLIKHGAKLLFAYSQATVPLVTIITRKAFGGAYIVMASKHVGADLNYAWPTAQIAVMGAKGAVEIIFRAEIGDADKVAERTKEYEDRFLSPFVAAERGYIDEVIMPHSTRKRIARALGMLRTKEMEQPRKKHDNIPL

SEQ ID NO: 3 - protein, 510 aa, synthetic construct
MKDILEKLEERRAQARLGGREKRLEAQHKRGKLTARERIELLLDHGSFEEFDMFVQHRSTDFGMEKQKIPGDGVVTGWGTVNGRTVFLFSKDFTVFGGSSSEAHAAKIVKVQDMALKMRAPIIGIFDAGGARIQEGVAALGGHGEVFRRNVAASGVIPQISVIMGPCAGGDVYSPAMTDFIFMVRDTSYMFVTGPDVVKTVTNEVVTAEELGGAKVHTSKSSIADGSFENDVEAILQIRRLLDFLPANNIEGVPEIESFDDVNRLDKSLDTLIPDNPNKPYDMGELIRRVVDEGDFFEIQAAYARNIITGFGRVEGRTVGFVANQPLVLAGVLDSDASRKAARFVRFCNAFSIPIVTFVDVPGFLPGTAQEYGGLIKHGAKLLFAYSQATVPLVTIITRKAFGGAYIVMASKHVGADLNYAWPTAQIAVMGAKGAVEIIFRAEIGDADKVAERTKEYEDRFLSPFVAAERGYIDEVIMPHSTRKRIARALGMLRTKEMEQPRKKHDNIPL

SEQ ID NO: 4 - protein, 510 aa, synthetic construct
MKDILEKLEERRAQARLGGGEKRLEAQHKRGKLTARERIELLLDHGSFEEFDEFVQHRSTDFGMEKQKIPGDGVVTGWGTVNGRTVFLFSKDFTVFGGSSSEAHAAKIVKVQDMALKMRAPIIGIFDAGGARIQEGVAALGGHGEVFRRNVAASGVIPQISVIMGPCAGGDVYSPAMTDFIFMVRDTSYMFVTGPDVVKTVTNEVVTAEELGGAKVHTSKSSIADGSFENDVEAILQIRRLLDFLPANNIEGVPEIESFDDVNRLDKSLDTLIPDNPNKPYDMGELIRRVVDEGDFFEIQAAYARNIITGFGRVEGRTVGFVANQPLVLAGVLDSDASRKAARFVRFCNAFSIPIVTFVDVPGFLPGTAQEYGGLIKHGAKLLFAYSQATVPLVTIITRKAFGGAYIVMASKHVGADLNYAWPTAQIAVMGAKGAVEIIFRAEIGDADKVAERTKEYEDRFLSPFVAAERGYIDEVIMPHSTRKRIARALGMLRTKEMEQPRKKHDNIPL

SEQ ID NO: 5 - protein, 510 aa, synthetic construct
MKDILEKLEERRAQARLGGGEKRLEAQHKRGKLTARERIELLLDHGSFEEFDQFVQHRSTDFGMEKQKIPGDGVVTGWGTVNGRTVFLFSKDFTVFGGSSSEAHAAKIVKVQDMALKMRAPIIGIFDAGGARIQEGVAALGGHGEVFRRNVAASGVIPQISVIMGPCAGGDVYSPAMTDFIFMVRDTSYMFVTGPDVVKTVTNEVVTAEELGGAKVHTSKSSIADGSFENDVEAILQIRRLLDFLPANNIEGVPEIESFDDVNRLDKSLDTLIPDNPNKPYDMGELIRRVVDEGDFFEIQAAYARNIITGFGRVEGRTVGFVANQPLVLAGVLDSDASRKAARFVRFCNAFSIPIVTFVDVPGFLPGTAQEYGGLIKHGAKLLFAYSQATVPLVTIITRKAFGGAYIVMASKHVGADLNYAWPTAQIAVMGAKGAVEIIFRAEIGDADKVAERTKEYEDRFLSPFVAAERGYIDEVIMPHSTRKRIARALGMLRTKEMEQPRKKHDNIPL

SEQ ID NO: 6 - protein, 510 aa, synthetic construct
MKDILEKLEERRAQARLGGGEKRLEAQHKRGKLTARERIELLLDHGSFEEFDMFVQHRSTDFGREKQKIPGDGVVTGWGTVNGRTVFLFSKDFTVFGGSSSEAHAAKIVKVQDMALKMRAPIIGIFDAGGARIQEGVAALGGHGEVFRRNVAASGVIPQISVIMGPCAGGDVYSPAMTDFIFMVRDTSYMFVTGPDVVKTVTNEVVTAEELGGAKVHTSKSSIADGSFENDVEAILQIRRLLDFLPANNIEGVPEIESFDDVNRLDKSLDTLIPDNPNKPYDMGELIRRVVDEGDFFEIQAAYARNIITGFGRVEGRTVGFVANQPLVLAGVLDSDASRKAARFVRFCNAFSIPIVTFVDVPGFLPGTAQEYGGLIKHGAKLLFAYSQATVPLVTIITRKAFGGAYIVMASKHVGADLNYAWPTAQIAVMGAKGAVEIIFRAEIGDADKVAERTKEYEDRFLSPFVAAERGYIDEVIMPHSTRKRIARALGMLRTKEMEQPRKKHDNIPL

SEQ ID NO: 7 - protein, 510 aa, synthetic construct
MKDILEKLEERRAQARLGGGEKRLEAQHKRGKLTARERIELLLDHGSFEEFDMFVQHRSTDFGMEKQKIPGDGVVTGWGTVNGRTVFLFSKDFTVFGGSNSEAHAAKIVKVQDMALKMRAPIIGIFDAGGARIQEGVAALGGHGEVFRRNVAASGVIPQISVIMGPCAGGDVYSPAMTDFIFMVRDTSYMFVTGPDVVKTVTNEVVTAEELGGAKVHTSKSSIADGSFENDVEAILQIRRLLDFLPANNIEGVPEIESFDDVNRLDKSLDTLIPDNPNKPYDMGELIRRVVDEGDFFEIQAAYARNIITGFGRVEGRTVGFVANQPLVLAGVLDSDASRKAARFVRFCNAFSIPIVTFVDVPGFLPGTAQEYGGLIKHGAKLLFAYSQATVPLVTIITRKAFGGAYIVMASKHVGADLNYAWPTAQIAVMGAKGAVEIIFRAEIGDADKVAERTKEYEDRFLSPFVAAERGYIDEVIMPHSTRKRIARALGMLRTKEMEQPRKKHDNIPL

SEQ ID NO: 8 - protein, 510 aa, synthetic construct
MKDILEKLEERRAQARLGGGEKRLEAQHKRGKLTARERIELLLDHGSFEEFDMFVQHRSTDFGMEKQKIPGDGVVTGWGTVNGRTVFLFSKDFTVFGGSSSEAHAAKIVKVQDMALKMRAPIIGIFDAGGARIQEGVAALGGHGEVFRRNVAASGVIPQISVIMGPCAGGDVYSPAMTDFIFMVRDTSYMFVTGPDVVKTVTNEVVTAEELGGAKVHTSKSSIADGSFENDVEAILQIRRLLDFLPANNIEGVPEIESFDDVNRLDKSLDTLIPDNPNKPYDMGELIRRVVDEGDFFEIQAAYARNIITGFGRVEGRTVGFVANQPLVLAGVLDSDASRKAARFVRFCNAFSIPIVTFVDVPGFLPGTAQEYGGLIKHGAKLLFAYSQATVPLVTIITRKAFGGAYVVMASKHVGADLNYAWPTAQIAVMGAKGAVEIIFRAEIGDADKVAERTKEYEDRFLSPFVAAERGYIDEVIMPHSTRKRIARALGMLRTKEMEQPRKKHDNIPL

SEQ ID NO: 9 - protein, 510 aa, synthetic construct
MKDILEKLEERRAQARLGGGEKRLEAQHKRGKLTARERIELLLDHGSFEEFDMFVQHRSTDFGMEKQKIPGDGVVTGWGTVNGRTVFLFSKDFTVFGGSSSEAHAAKIVKVQDMALKMRAPIIGIFDAGGARIQEGVAALGGHGEVFRRNVAASGVIPQISVIMGPCAGGDVYSPAMTDFIFMVRDTSYMFVTGPDVVKTVTNEVVTAEELGGAKVHTSKSSIADGSFENDVEAILQIRRLLDFLPANNIEGVPEIESFDDVNRLDKSLDTLIPDNPNKPYDMGELIRRVVDEGDFFEIQAAYARNIITGFGRVEGRTVGFVANQPLVLAGVLDSDASRKAARFVRFCNAFSIPIVTFVDVPGFLPGTAQEYGGLIKHGAKLLFAYSQATVPLVTIITRKAFGGAYIVMASKHVGADLNYAWPTAQIAVMGAKGAVEIIFRAEIGDADKVAERTKEYEDRFLSPFVAAERGYIDEVIMPHSTRKRIARALGMLRNKEMEQPRKKHDNIPL

SEQ ID NO: 10 - protein, 510 aa, synthetic construct
MKDILEKLEERRAQARLGGGEKRLEAQHKRGKLTARERIELLLDHGSFEEFDMFVQHRSTDFGMEKQKIPGDGVVTGWGTVNGRTVFLFSKDFTVFGGSSSEAHAAKIVKVQDMALKMRAPIIGIFDAGGARIQEGVAALGGHGEVFRRNVAASGVIPQISVIMGPCAGGDVYSPAMTDFIFMVRDTSYMFVTGPDVVKTVTNEVVTAEELGGAKVHTSKSSIADGSFENDVEAILQIRRLLDFLPANNIEGVPEIESFDDVNRLDKSLDTLIPDNPNKPYDMGELIRRVVDEGDFFEIQAAYARNIITGFGRVEGRTVGFVANQPLVLAGVLDSDASRKAARFVRFCNAFSIPIVTFVDVPGFLPGTAQEYGGLIKHGAKLLFAYSQATVPLVTIITRKAFGGAYIVMASKHVGADLNYAWPTAQIAVMGAKGAVEIIFRAEIGDADKVAERTKEYEDRFLSPFVAAERGYIDEVIMPHSTRKRIARALGMLRTKEMEQPHKKHDNIPL

SEQ ID NO: 11 - protein, 510 aa, synthetic construct
MKDILEKLEERRAQARLGGGEKRLEAQHKRGKLTARERIELLLDHGSFEEFDMFVQHRSTDFGMEKQKIPGDGVVTGWGTVNGRTVFLFSKDFTVFGGSSSEAHAAKIVKVQDMALKMRAPIIGIFDAGGARIQEGVAALGGHGEVFRRNVAASGVIPQISVIMGPCAGGDVYSPAMTDFIFMVRDTSYMFVTGPDVVKTVTNEVVTAEELGGAKVHTSKSSIADGSFENDVEAILQIRRLLDFLPANNIEGVPEIESFDDVNRLDKSLDTLIPDNPNKPYDMGELIRRVVDEGDFFEIQAAYARNIITGFGRVEGRTVGFVANQPLVLAGVLDSDASRKAARFVRFCNAFSIPIVTFVDVPGFLPGTAQEYGGLIKHGAKLLFAYSQATVPLVTIITRKAFGGAYIVMASKHVGADLNYAWPTAQIAVMGAKGAVEIIFRAEIGDADKVAERTKEYEDRFLSPFVAAERGYIDEVIMPHSTRKRIARALGMLRTKEMEQPRKKHDNIPF

SEQ ID NO: 12 - protein, 510 aa, Methylorubrum extorquens
MKDILEKLEERRAQARLGGGEKRLEAQHKRGKLTARERIELLLDHGSFEEFDMFVQHRSTDFGMEKQKIPGDGVVTGWGTVNGRTVFLFSKDFTVFGGSLSEAHAAKIVKVQDMALKMRAPIIGIFDAGGARIQEGVAALGGYGEVFRRNVAASGVIPQISVIMGPCAGGDVYSPAMTDFIFMVRDTSYMFVTGPDVVKTVTNEVVTAEELGGAKVHTSKSSIADGSFENDVEAILQIRRLLDFLPANNIEGVPEIESFDDVNRLDKSLDTLIPDNPNKPYDMGELIRRVVDEGDFFEIQAAYARNIITGFGRVEGRTVGFVANQPLVLAGVLDSDASRKAARFVRFCNAFSIPIVTFVDVPGFLPGTAQEYGGLIKHGAKLLFAYSQATVPLVTIITRKAFGGAYDVMASKHVGADLNYAWPTAQIAVMGAKGAVEIIFRAEIGDADKIAERTKEYEDRFLSPFVAAERGYIDEVIMPHSTRKRIARALGMLRTKEMEQPWKKHDNIPL

SEQ ID NO: 13 - protein, 667 aa, Methylorubrum extorquens
MFDKILIANRGEIACRIIKTAQKMGIKTVAVYSDADRDAVHVAMADEAVHIGPAPAAQSYLLIEKIIDACKQTGAQAVHPGYGFLSERESFPKALAEAGIVFIGPNPGAIAAMGDKIESKKAAAAAEVSTVPGFLGVIESPEHAVTIADEIGYPVMIKASAGGGGKGMRIAESADEVAEGFARAKSEASSSFGDDRVFVEKFITDPRHIEIQVIGDKHGNVIYLGERECSIQRRNQKVIEEAPSPLLDEETRRKMGEQAVALAKAVNYDSAGTVEFVAGQDKSFYFLEMNTRLQVEHPVTEMITGLDLVELMIRVAAGEKLPLSQDQVKLDGWAVESRVYAEDPTRNFLPSIGRLTTYQPPEEGPLGGAIVRNDTGVEEGGEIAIHYDPMIAKLVTWAPTRLEAIEAQATALDAFAIEGIRHNIPFLATLMAHPRWRDGRLSTGFIKEEFPEGFIAPEPEGPVAHRLAAVAAAIDHKLNIRKRGISGQMRDPSLLTFQRERVVVLSGQRFNVTVDPDGDDLLVTFDDGTTAPVRSAWRPGAPVWSGTVGDQSVAIQVRPLLNGVFLQHAGAAAEARVFTRREAELADLMPVKENAGSGKQLLCPMPGLVKQIMVSEGQEVKNGEPLAIVEAMKMENVLRAERDGTISKIAAKEGDSLAVDAVILEFA

SEQ ID NO: 14 - DNA, 1533 bp, synthetic construct
  source 1..1533: mol_type = other DNA
atgaaggacatcctcgagaagcttgaggagcgtcgcgcacaggcccgtctcggcggcggggaaaagcggctcgaggcgcagcacaagcgcggcaagctcacggcgcgcgagcgcatcgaactcctgctcgaccacgggtcgttcgaggagttcgacatgttcgtgcagcaccgctccaccgatttcggcatggagaagcagaagatccccggcgacggcgtcgtcaccggctggggcaccgtgaacgggcgcaccgtcttcctgttctcgaaggacttcacggtgttcggcggctcgtcctccgaggcgcacgcagccaagatcgttaaggtccaggacatggcgctgaagatgcgcgccccgatcatcggcatcttcgatgccggcggtgcgcgcatccaggagggcgtggccgcgctcggcggccacggcgaggtgttccgccgcaacgtcgctgcctccggcgtgatcccgcagatctcggtcatcatggggccgtgcgcgggcggcgacgtgtactcgccggccatgaccgacttcatcttcatggtgcgtgacacgagctacatgttcgtgaccggccccgacgtggtgaagaccgtcaccaacgaggtcgtgaccgccgaggaactcggcggcgccaaggtccacacctcgaaatcctcgatcgccgacggctcgttcgagaacgacgtcgaggcgatcctccagatccgccgcctgctcgacttcctgcccgcgaacaacatcgagggcgtgccggagatcgagagcttcgacgacgtcaaccgcctcgacaagtcgctcgacacgctgatcccggacaacccgaacaagccctacgacatgggcgagctgatccgccgggtcgtggacgaaggcgacttcttcgagatccaggcggcttacgcccgcaatattatcaccggcttcggccgcgtcgagggccgcaccgtcggtttcgtcgccaaccagccgctggtgctggccggcgtgcttgattcggacgcctcccggaaggcggcccgcttcgtgcgcttctgcaacgccttctcgatcccgatcgtcaccttcgtggacgtgccgggcttcctgccgggcacggcgcaggaatatggcggcctgatcaagcacggcgccaagctgctcttcgcctacagccaagccaccgtgccgctcgtgaccatcatcacccgcaaggccttcggcggcgcctacatcgtcatggcctccaagcatgtcggcgccgacctgaactacgcgtggccgacggcgcagatcgcggtgatgggcgccaagggcgctgtcgagatcatcttccgcgccgagatcggcgatgcggacaaggtcgccgagcggaccaaagaatacgaggaccgcttcctctcgcccttcgtggcggcggagcgcggctacatcgacgaggtgatcatgccccactccacccgcaagcggatcgcgcgggcgctcgggatgctgcgcaccaaggagatggagcagcccaggaagaagcacgacaacatcccgctctga

SEQ ID NO: 15 - DNA, 1533 bp, synthetic construct
  source 1..1533: mol_type = other DNA
atgaaggacatcctcgagaagtttgaggagcgtcgcgcacaggcccgtctcggcggcggggaaaagcggctcgaggcgcagcacaagcgcggcaagctcacggcgcgcgagcgcatcgaactcctgctcgaccacgggtcgttcgaggagttcgacatgttcgtgcagcaccgctccaccgatttcggcatggagaagcagaagatccccggcgacggcgtcgtcaccggctggggcaccgtgaacgggcgcaccgtcttcctgttctcgaaggacttcacggtgttcggcggctcgtcctccgaggcgcacgcagccaagatcgttaaggtccaggacatggcgctgaagatgcgcgccccgatcatcggcatcttcgatgccggcggtgcgcgcatccaggagggcgtggccgcgctcggcggccacggcgaggtgttccgccgcaacgtcgctgcctccggcgtgatcccgcagatctcggtcatcatggggccgtgcgcgggcggcgacgtgtactcgccggccatgaccgacttcatcttcatggtgcgtgacacgagctacatgttcgtgaccggccccgacgtggtgaagaccgtcaccaacgaggtcgtgaccgccgaggaactcggcggcgccaaggtccacacctcgaaatcctcgatcgccgacggctcgttcgagaacgacgtcgaggcgatcctccagatccgccgcctgctcgacttcctgcccgcgaacaacatcgagggcgtgccggagatcgagagcttcgacgacgtcaaccgcctcgacaagtcgctcgacacgctgatcccggacaacccgaacaagccctacgacatgggcgagctgatccgccgggtcgtggacgaaggcgacttcttcgagatccaggcggcttacgcccgcaatattatcaccggcttcggccgcgtcgagggccgcaccgtcggtttcgtcgccaaccagccgctggtgctggccggcgtgcttgattcggacgcctcccggaaggcggcccgcttcgtgcgcttctgcaacgccttctcgatcccgatcgtcaccttcgtggacgtgccgggcttcctgccgggcacggcgcaggaatatggcggcctgatcaagcacggcgccaagctgctcttcgcctacagccaagccaccgtgccgctcgtgaccatcatcacccgcaaggccttcggcggcgcctacatcgtcatggcctccaagcatgtcggcgccgacctgaactacgcgtggccgacggcgcagatcgcggtgatgggcgccaagggcgctgtcgagatcatcttccgcgccgagatcggcgatgcggacaaggtcgccgagcggaccaaagaatacgaggaccgcttcctctcgcccttcgtggcggcggagcgcggctacatcgacgaggtgatcatgccccactccacccgcaagcggatcgcgcgggcgctcgggatgctgcgcaccaaggagatggagcagcccaggaagaagcacgacaacatcccgctctga

SEQ ID NO: 16 - DNA, 1533 bp, synthetic construct
  source 1..1533: mol_type = other DNA
atgaaggacatcctcgagaagcttgaggagcgtcgcgcacaggcccgtctcggcggccgtgaaaagcggctcgaggcgcagcacaagcgcggcaagctcacggcgcgcgagcgcatcgaactcctgctcgaccacgggtcgttcgaggagttcgacatgttcgtgcagcaccgctccaccgatttcggcatggagaagcagaagatccccggcgacggcgtcgtcaccggctggggcaccgtgaacgggcgcaccgtcttcctgttctcgaaggacttcacggtgttcggcggctcgtcctccgaggcgcacgcagccaagatcgttaaggtccaggacatggcgctgaagatgcgcgccccgatcatcggcatcttcgatgccggcggtgcgcgcatccaggagggcgtggccgcgctcggcggccacggcgaggtgttccgccgcaacgtcgctgcctccggcgtgatcccgcagatctcggtcatcatggggccgtgcgcgggcggcgacgtgtactcgccggccatgaccgacttcatcttcatggtgcgtgacacgagctacatgttcgtgaccggccccgacgtggtgaagaccgtcaccaacgaggtcgtgaccgccgaggaactcggcggcgccaaggtccacacctcgaaatcctcgatcgccgacggctcgttcgagaacgacgtcgaggcgatcctccagatccgccgcctgctcgacttcctgcccgcgaacaacatcgagggcgtgccggagatcgagagcttcgacgacgtcaaccgcctcgacaagtcgctcgacacgctgatcccggacaacccgaacaagccctacgacatgggcgagctgatccgccgggtcgtggacgaaggcgacttcttcgagatccaggcggcttacgcccgcaatattatcaccggcttcggccgcgtcgagggccgcaccgtcggtttcgtcgccaaccagccgctggtgctggccggcgtgcttgattcggacgcctcccggaaggcggcccgcttcgtgcgcttctgcaacgccttctcgatcccgatcgtcaccttcgtggacgtgccgggcttcctgccgggcacggcgcaggaatatggcggcctgatcaagcacggcgccaagctgctcttcgcctacagccaagccaccgtgccgctcgtgaccatcatcacccgcaaggccttcggcggcgcctacatcgtcatggcctccaagcatgtcggcgccgacctgaactacgcgtggccgacggcgcagatcgcggtgatgggcgccaagggcgctgtcgagatcatcttccgcgccgagatcggcgatgcggacaaggtcgccgagcggaccaaagaatacgaggaccgcttcctctcgcccttcgtggcggcggagcgcggctacatcgacgaggtgatcatgccccactccacccgcaagcggatcgcgcgggcgctcgggatgctgcgcaccaaggagatggagcagcccaggaagaagcacgacaacatcccgctctga

SEQ ID NO: 17 - DNA, 1533 bp, synthetic construct
  source 1..1533: mol_type = other DNA
atgaaggacatcctcgagaagcttgaggagcgtcgcgcacaggcccgtctcggcggcggggaaaagcggctcgaggcgcagcacaagcgcggcaagctcacggcgcgcgagcgcatcgaactcctgctcgaccacgggtcgttcgaggagttcgacgaattcgtgcagcaccgctccaccgatttcggcatggagaagcagaagatccccggcgacggcgtcgtcaccggctggggcaccgtgaacgggcgcaccgtcttcctgttctcgaaggacttcacggtgttcggcggctcgtcctccgaggcgcacgcagccaagatcgttaaggtccaggacatggcgctgaagatgcgcgccccgatcatcggcatcttcgatgccggcggtgcgcgcatccaggagggcgtggccgcgctcggcggccacggcgaggtgttccgccgcaacgtcgctgcctccggcgtgatcccgcagatctcggtcatcatggggccgtgcgcgggcggcgacgtgtactcgccggccatgaccgacttcatcttcatggtgcgtgacacgagctacatgttcgtgaccggccccgacgtggtgaagaccgtcaccaacgaggtcgtgaccgccgaggaactcggcggcgccaaggtccacacctcgaaatcctcgatcgccgacggctcgttcgagaacgacgtcgaggcgatcctccagatccgccgcctgctcgacttcctgcccgcgaacaacatcgagggcgtgccggagatcgagagcttcgacgacgtcaaccgcctcgacaagtcgctcgacacgctgatcccggacaacccgaacaagccctacgacatgggcgagctgatccgccgggtcgtggacgaaggcgacttcttcgagatccaggcggcttacgcccgcaatattatcaccggcttcggccgcgtcgagggccgcaccgtcggtttcgtcgccaaccagccgctggtgctggccggcgtgcttgattcggacgcctcccggaaggcggcccgcttcgtgcgcttctgcaacgccttctcgatcccgatcgtcaccttcgtggacgtgccgggcttcctgccgggcacggcgcaggaatatggcggcctgatcaagcacggcgccaagctgctcttcgcctacagccaagccaccgtgccgctcgtgaccatcatcacccgcaaggccttcggcggcgcctacatcgtcatggcctccaagcatgtcggcgccgacctgaactacgcgtggccgacggcgcagatcgcggtgatgggcgccaagggcgctgtcgagatcatcttccgcgccgagatcggcgatgcggacaaggtcgccgagcggaccaaagaatacgaggaccgcttcctctcgcccttcgtggcggcggagcgcggctacatcgacgaggtgatcatgccccactccacccgcaagcggatcgcgcgggcgctcgggatgctgcgcaccaaggagatggagcagcccaggaagaagcacgacaacatcccgctctga

SEQ ID NO: 18 - DNA, 1533 bp, synthetic construct
  source 1..1533: mol_type = other DNA
atgaaggacatcctcgagaagcttgaggagcgtcgcgcacaggcccgtctcggcggcggggaaaagcggctcgaggcgcagcacaagcgcggcaagctcacggcgcgcgagcgcatcgaactcctgctcgaccacgggtcgttcgaggagttcgaccagttcgtgcagcaccgctccaccgatttcggcatggagaagcagaagatccccggcgacggcgtcgtcaccggctggggcaccgtgaacgggcgcaccgtcttcctgttctcgaaggacttcacggtgttcggcggctcgtcctccgaggcgcacgcagccaagatcgttaaggtccaggacatggcgctgaagatgcgcgccccgatcatcggcatcttcgatgccggcggtgcgcgcatccaggagggcgtggccgcgctcggcggccacggcgaggtgttccgccgcaacgtcgctgcctccggcgtgatcccgcagatctcggtcatcatggggccgtgcgcgggcggcgacgtgtactcgccggccatgaccgacttcatcttcatggtgcgtgacacgagctacatgttcgtgaccggccccgacgtggtgaagaccgtcaccaacgaggtcgtgaccgccgaggaactcggcggcgccaaggtccacacctcgaaatcctcgatcgccgacggctcgttcgagaacgacgtcgaggcgatcctccagatccgccgcctgctcgacttcctgcccgcgaacaacatcgagggcgtgccggagatcgagagcttcgacgacgtcaaccgcctcgacaagtcgctcgacacgctgatcccggacaacccgaacaagccctacgacatgggcgagctgatccgccgggtcgtggacgaaggcgacttcttcgagatccaggcggcttacgcccgcaatattatcaccggcttcggccgcgtcgagggccgcaccgtcggtttcgtcgccaaccagccgctggtgctggccggcgtgcttgattcggacgcctcccggaaggcggcccgcttcgtgcgcttctgcaacgccttctcgatcccgatcgtcaccttcgtggacgtgccgggcttcctgccgggcacggcgcaggaatatggcggcctgatcaagcacggcgccaagctgctcttcgcctacagccaagccaccgtgccgctcgtgaccatcatcacccgcaaggccttcggcggcgcctacatcgtcatggcctccaagcatgtcggcgccgacctgaactacgcgtggccgacggcgcagatcgcggtgatgggcgccaagggcgctgtcgagatcatcttccgcgccgagatcggcgatgcggacaaggtcgccgagcggaccaaagaatacgaggaccgcttcctctcgcccttcgtggcggcggagcgcggctacatcgacgaggtgatcatgccccactccacccgcaagcggatcgcgcgggcgctcgggatgctgcgcaccaaggagatggagcagcccaggaagaagcacgacaacatcccgctctga

SEQ ID NO: 19 - DNA, 1533 bp, synthetic construct
  source 1..1533: mol_type = other DNA
atgaaggacatcctcgagaagcttgaggagcgtcgcgcacaggcccgtctcggcggcggggaaaagcggctcgaggcgcagcacaagcgcggcaagctcacggcgcgcgagcgcatcgaactcctgctcgaccacgggtcgttcgaggagttcgacatgttcgtgcagcaccgctccaccgatttcggccgtgagaagcagaagatccccggcgacggcgtcgtcaccggctggggcaccgtgaacgggcgcaccgtcttcctgttctcgaaggacttcacggtgttcggcggctcgtcctccgaggcgcacgcagccaagatcgttaaggtccaggacatggcgctgaagatgcgcgccccgatcatcggcatcttcgatgccggcggtgcgcgcatccaggagggcgtggccgcgctcggcggccacggcgaggtgttccgccgcaacgtcgctgcctccggcgtgatcccgcagatctcggtcatcatggggccgtgcgcgggcggcgacgtgtactcgccggccatgaccgacttcatcttcatggtgcgtgacacgagctacatgttcgtgaccggccccgacgtggtgaagaccgtcaccaacgaggtcgtgaccgccgaggaactcggcggcgccaaggtccacacctcgaaatcctcgatcgccgacggctcgttcgagaacgacgtcgaggcgatcctccagatccgccgcctgctcgacttcctgcccgcgaacaacatcgagggcgtgccggagatcgagagcttcgacgacgtcaaccgcctcgacaagtcgctcgacacgctgatcccggacaacccgaacaagccctacgacatgggcgagctgatccgccgggtcgtggacgaaggcgacttcttcgagatccaggcggcttacgcccgcaatattatcaccggcttcggccgcgtcgagggccgcaccgtcggtttcgtcgccaaccagccgctggtgctggccggcgtgcttgattcggacgcctcccggaaggcggcccgcttcgtgcgcttctgcaacgccttctcgatcccgatcgtcaccttcgtggacgtgccgggcttcctgccgggcacggcgcaggaatatggcggcctgatcaagcacggcgccaagctgctcttcgcctacagccaagccaccgtgccgctcgtgaccatcatcacccgcaaggccttcggcggcgcctacatcgtcatggcctccaagcatgtcggcgccgacctgaactacgcgtggccgacggcgcagatcgcggtgatgggcgccaagggcgctgtcgagatcatcttccgcgccgagatcggcgatgcggacaaggtcgccgagcggaccaaagaatacgaggaccgcttcctctcgcccttcgtggcggcggagcgcggctacatcgacgaggtgatcatgccccactccacccgcaagcggatcgcgcgggcgctcgggatgctgcgcaccaaggagatggagcagcccaggaagaagcacgacaacatcccgctctga

SEQ ID NO: 20 - DNA, 1533 bp, synthetic construct
  source 1..1533: mol_type = other DNA
atgaaggacatcctcgagaagcttgaggagcgtcgcgcacaggcccgtctcggcggcggggaaaagcggctcgaggcgcagcacaagcgcggcaagctcacggcgcgcgagcgcatcgaactcctgctcgaccacgggtcgttcgaggagttcgacatgttcgtgcagcaccgctccaccgatttcggcatggagaagcagaagatccccggcgacggcgtcgtcaccggctggggcaccgtgaacgggcgcaccgtcttcctgttctcgaaggacttcacggtgttcggcggctcgaactccgaggcgcacgcagccaagatcgttaaggtccaggacatggcgctgaagatgcgcgccccgatcatcggcatcttcgatgccggcggtgcgcgcatccaggagggcgtggccgcgctcggcggccacggcgaggtgttccgccgcaacgtcgctgcctccggcgtgatcccgcagatctcggtcatcatggggccgtgcgcgggcggcgacgtgtactcgccggccatgaccgacttcatcttcatggtgcgtgacacgagctacatgttcgtgaccggccccgacgtggtgaagaccgtcaccaacgaggtcgtgaccgccgaggaactcggcggcgccaaggtccacacctcgaaatcctcgatcgccgacggctcgttcgagaacgacgtcgaggcgatcctccagatccgccgcctgctcgacttcctgcccgcgaacaacatcgagggcgtgccggagatcgagagcttcgacgacgtcaaccgcctcgacaagtcgctcgacacgctgatcccggacaacccgaacaagccctacgacatgggcgagctgatccgccgggtcgtggacgaaggcgacttcttcgagatccaggcggcttacgcccgcaatattatcaccggcttcggccgcgtcgagggccgcaccgtcggtttcgtcgccaaccagccgctggtgctggccggcgtgcttgattcggacgcctcccggaaggcggcccgcttcgtgcgcttctgcaacgccttctcgatcccgatcgtcaccttcgtggacgtgccgggcttcctgccgggcacggcgcaggaatatggcggcctgatcaagcacggcgccaagctgctcttcgcctacagccaagccaccgtgccgctcgtgaccatcatcacccgcaaggccttcggcggcgcctacatcgtcatggcctccaagcatgtcggcgccgacctgaactacgcgtggccgacggcgcagatcgcggtgatgggcgccaagggcgctgtcgagatcatcttccgcgccgagatcggcgatgcggacaaggtcgccgagcggaccaaagaatacgaggaccgcttcctctcgcccttcgtggcggcggagcgcggctacatcgacgaggtgatcatgccccactccacccgcaagcggatcgcgcgggcgctcgggatgctgcgcaccaaggagatggagcagcccaggaagaagcacgacaacatcccgctctga

SEQ ID NO: 21 - DNA, 1533 bp, synthetic construct
  source 1..1533: mol_type = other DNA
atgaaggacatcctcgagaagcttgaggagcgtcgcgcacaggcccgtctcggcggcggggaaaagcggctcgaggcgcagcacaagcgcggcaagctcacggcgcgcgagcgcatcgaactcctgctcgaccacgggtcgttcgaggagttcgacatgttcgtgcagcaccgctccaccgatttcggcatggagaagcagaagatccccggcgacggcgtcgtcaccggctggggcaccgtgaacgggcgcaccgtcttcctgttctcgaaggacttcacggtgttcggcggctcgtcctccgaggcgcacgcagccaagatcgttaaggtccaggacatggcgctgaagatgcgcgccccgatcatcggcatcttcgatgccggcggtgcgcgcatccaggagggcgtggccgcgctcggcggccacggcgaggtgttccgccgcaacgtcgctgcctccggcgtgatcccgcagatctcggtcatcatggggccgtgcgcgggcggcgacgtgtactcgccggccatgaccgacttcatcttcatggtgcgtgacacgagctacatgttcgtgaccggccccgacgtggtgaagaccgtcaccaacgaggtcgtgaccgccgaggaactcggcggcgccaaggtccacacctcgaaatcctcgatcgccgacggctcgttcgagaacgacgtcgaggcgatcctccagatccgccgcctgctcgacttcctgcccgcgaacaacatcgagggcgtgccggagatcgagagcttcgacgacgtcaaccgcctcgacaagtcgctcgacacgctgatcccggacaacccgaacaagccctacgacatgggcgagctgatccgccgggtcgtggacgaaggcgacttcttcgagatccaggcggcttacgcccgcaatattatcaccggcttcggccgcgtcgagggccgcaccgtcggtttcgtcgccaaccagccgctggtgctggccggcgtgcttgattcggacgcctcccggaaggcggcccgcttcgtgcgcttctgcaacgccttctcgatcccgatcgtcaccttcgtggacgtgccgggcttcctgccgggcacggcgcaggaatatggcggcctgatcaagcacggcgccaagctgctcttcgcctacagccaagccaccgtgccgctcgtgaccatcatcacccgcaaggccttcggcggcgcctacgtcgtcatggcctccaagcatgtcggcgccgacctgaactacgcgtggccgacggcgcagatcgcggtgatgggcgccaagggcgctgtcgagatcatcttccgcgccgagatcggcgatgcggacaaggtcgccgagcggaccaaagaatacgaggaccgcttcctctcgcccttcgtggcggcggagcgcggctacatcgacgaggtgatcatgccccactccacccgcaagcggatcgcgcgggcgctcgggatgctgcgcaccaaggagatggagcagcccaggaagaagcacgacaacatcccgctctga

SEQ ID NO: 22 - DNA, 1533 bp, synthetic construct
  source 1..1533: mol_type = other DNA
atgaaggacatcctcgagaagcttgaggagcgtcgcgcacaggcccgtctcggcggcggggaaaagcggctcgaggcgcagcacaagcgcggcaagctcacggcgcgcgagcgcatcgaactcctgctcgaccacgggtcgttcgaggagttcgacatgttcgtgcagcaccgctccaccgatttcggcatggagaagcagaagatccccggcgacggcgtcgtcaccggctggggcaccgtgaacgggcgcaccgtcttcctgttctcgaaggacttcacggtgttcggcggctcgtcctccgaggcgcacgcagccaagatcgttaaggtccaggacatggcgctgaagatgcgcgccccgatcatcggcatcttcgatgccggcggtgcgcgcatccaggagggcgtggccgcgctcggcggccacggcgaggtgttccgccgcaacgtcgctgcctccggcgtgatcccgcagatctcggtcatcatggggccgtgcgcgggcggcgacgtgtactcgccggccatgaccgacttcatcttcatggtgcgtgacacgagctacatgttcgtgaccggccccgacgtggtgaagaccgtcaccaacgaggtcgtgaccgccgaggaactcggcggcgccaaggtccacacctcgaaatcctcgatcgccgacggctcgttcgagaacgacgtcgaggcgatcctccagatccgccgcctgctcgacttcctgcccgcgaacaacatcgagggcgtgccggagatcgagagcttcgacgacgtcaaccgcctcgacaagtcgctcgacacgctgatcccggacaacccgaacaagccctacgacatgggcgagctgatccgccgggtcgtggacgaaggcgacttcttcgagatccaggcggcttacgcccgcaatattatcaccggcttcggccgcgtcgagggccgcaccgtcggtttcgtcgccaaccagccgctggtgctggccggcgtgcttgattcggacgcctcccggaaggcggcccgcttcgtgcgcttctgcaacgccttctcgatcccgatcgtcaccttcgtggacgtgccgggcttcctgccgggcacggcgcaggaatatggcggcctgatcaagcacggcgccaagctgctcttcgcctacagccaagccaccgtgccgctcgtgaccatcatcacccgcaaggccttcggcggcgcctacatcgtcatggcctccaagcatgtcggcgccgacctgaactacgcgtggccgacggcgcagatcgcggtgatgggcgccaagggcgctgtcgagatcatcttccgcgccgagatcggcgatgcggacaaggtcgccgagcggaccaaagaatacgaggaccgcttcctctcgcccttcgtggcggcggagcgcggctacatcgacgaggtgatcatgccccactccacccgcaagcggatcgcgcgggcgctcgggatgctgcgcaacaaggagatggagcagcccaggaagaagcacgacaacatcccgctctga

SEQ ID NO: 23 - DNA, 1533 bp, synthetic construct
  source 1..1533: mol_type = other DNA
atgaaggacatcctcgagaagcttgaggagcgtcgcgcacaggcccgtctcggcggcggggaaaagcggctcgaggcgcagcacaagcgcggcaagctcacggcgcgcgagcgcatcgaactcctgctcgaccacgggtcgttcgaggagttcgacatgttcgtgcagcaccgctccaccgatttcggcatggagaagcagaagatccccggcgacggcgtcgtcaccggctggggcaccgtgaacgggcgcaccgtcttcctgttctcgaaggacttcacggtgttcggcggctcgtcctccgaggcgcacgcagccaagatcgttaaggtccaggacatggcgctgaagatgcgcgccccgatcatcggcatcttcgatgccggcggtgcgcgcatccaggagggcgtggccgcgctcggcggccacggcgaggtgttccgccgcaacgtcgctgcctccggcgtgatcccgcagatctcggtcatcatggggccgtgcgcgggcggcgacgtgtactcgccggccatgaccgacttcatcttcatggtgcgtgacacgagctacatgttcgtgaccggccccgacgtggtgaagaccgtcaccaacgaggtcgtgaccgccgaggaactcggcggcgccaaggtccacacctcgaaatcctcgatcgccgacggctcgttcgagaacgacgtcgaggcgatcctccagatccgccgcctgctcgacttcctgcccgcgaacaacatcgagggcgtgccggagatcgagagcttcgacgacgtcaaccgcctcgacaagtcgctcgacacgctgatcccggacaacccgaacaagccctacgacatgggcgagctgatccgccgggtcgtggacgaaggcgacttcttcgagatccaggcggcttacgcccgcaatattatcaccggcttcggccgcgtcgagggccgcaccgtcggtttcgtcgccaaccagccgctggtgctggccggcgtgcttgattcggacgcctcccggaaggcggcccgcttcgtgcgcttctgcaacgccttctcgatcccgatcgtcaccttcgtggacgtgccgggcttcctgccgggcacggcgcaggaatatggcggcctgatcaagcacggcgccaagctgctcttcgcctacagccaagccaccgtgccgctcgtgaccatcatcacccgcaaggccttcggcggcgcctacatcgtcatggcctccaagcatgtcggcgccgacctgaactacgcgtggccgacggcgcagatcgcggtgatgggcgccaagggcgctgtcgagatcatcttccgcgccgagatcggcgatgcggacaaggtcgccgagcggaccaaagaatacgaggaccgcttcctctcgcccttcgtggcggcggagcgcggctacatcgacgaggtgatcatgccccactccacccgcaagcggatcgcgcgggcgctcgggatgctgcgcaccaaggagatggagcagccccataagaagcacgacaacatcccgctctga

SEQ ID NO: 24 - DNA, 1533 bp, synthetic construct
  source 1..1533: mol_type = other DNA
atgaaggacatcctcgagaagcttgaggagcgtcgcgcacaggcccgtctcggcggcggggaaaagcggctcgaggcgcagcacaagcgcggcaagctcacggcgcgcgagcgcatcgaactcctgctcgaccacgggtcgttcgaggagttcgacatgttcgtgcagcaccgctccaccgatttcggcatggagaagcagaagatccccggcgacggcgtcgtcaccggctggggcaccgtgaacgggcgcaccgtcttcctgttctcgaaggacttcacggtgttcggcggctcgtcctccgaggcgcacgcagccaagatcgttaaggtccaggacatggcgctgaagatgcgcgccccgatcatcggcatcttcgatgccggcggtgcgcgcatccaggagggcgtggccgcgctcggcggccacggcgaggtgttccgccgcaacgtcgctgcctccggcgtgatcccgcagatctcggtcatcatggggccgtgcgcgggcggcgacgtgtactcgccggccatgaccgacttcatcttcatggtgcgtgacacgagctacatgttcgtgaccggccccgacgtggtgaagaccgtcaccaacgaggtcgtgaccgccgaggaactcggcggcgccaaggtccacacctcgaaatcctcgatcgccgacggctcgttcgagaacgacgtcgaggcgatcctccagatccgccgcctgctcgacttcctgcccgcgaacaacatcgagggcgtgccggagatcgagagcttcgacgacgtcaaccgcctcgacaagtcgctcgacacgctgatcccggacaacccgaacaagccctacgacatgggcgagctgatccgccgggtcgtggacgaaggcgacttcttcgagatccaggcggcttacgcccgcaatattatcaccggcttcggccgcgtcgagggccgcaccgtcggtttcgtcgccaaccagccgctggtgctggccggcgtgcttgattcggacgcctcccggaaggcggcccgcttcgtgcgcttctgcaacgccttctcgatcccgatcgtcaccttcgtggacgtgccgggcttcctgccgggcacggcgcaggaatatggcggcctgatcaagcacggcgccaagctgctcttcgcctacagccaagccaccgtgccgctcgtgaccatcatcacccgcaaggccttcggcggcgcctacatcgtcatggcctccaagcatgtcggcgccgacctgaactacgcgtggccgacggcgcagatcgcggtgatgggcgccaagggcgctgtcgagatcatcttccgcgccgagatcggcgatgcggacaaggtcgccgagcggaccaaagaatacgaggaccgcttcctctcgcccttcgtggcggcggagcgcggctacatcgacgaggtgatcatgccccactccacccgcaagcggatcgcgcgggcgctcgggatgctgcgcaccaaggagatggagcagcccaggaagaagcacgacaacatcccgttctga

SEQ ID NO: 25 - DNA, 1533 bp, Methylorubrum extorquens
  source 1..1533: mol_type = genomic DNA
atgaaggacattctcgagaagcttgaggagcgtcgcgcacaggcccgtctcggcggcggggaaaagcggctcgaggcgcagcacaagcgcggcaagctcacggcgcgcgagcgcatcgaactcctgctcgaccacgggtcgttcgaggagttcgacatgttcgtgcagcaccgctccaccgatttcggcatggagaagcagaagatccccggcgacggcgtcgtcaccggctggggcaccgtgaacgggcgcaccgtcttcctgttctcgaaggacttcacggtgttcggcggctcgctctccgaggcgcacgcagccaagatcgttaaggtccaggacatggcgctgaagatgcgcgccccgatcatcggcatcttcgatgccggcggtgcgcgcatccaggagggcgtggccgcgctcggcggctacggcgaggtgttccgccgcaacgtcgctgcctccggcgtgatcccgcagatctcggtcatcatggggccgtgcgcgggcggcgacgtgtactcgccggccatgaccgacttcatcttcatggtgcgtgacacgagctacatgttcgtgaccggccccgacgtggtgaagaccgtcaccaacgaggtcgtgaccgccgaggaactcggcggcgccaaggtccacacctcgaaatcctcgatcgccgacggctcgttcgagaacgacgtcgaggcgatcctccagatccgccgcctgctcgacttcctgcccgcgaacaacatcgagggcgtgccggagatcgagagcttcgacgacgtcaaccgcctcgacaagtcgctcgacacgctgatcccggacaacccgaacaagccctacgacatgggcgagctgatccgccgggtcgtggacgaaggcgacttcttcgagatccaggcggcttacgcccgcaatattatcaccggcttcggccgcgtcgagggccgcaccgtcggtttcgtcgccaaccagccgctggtgctggccggcgtgcttgattcggacgcctcccggaaggcggcccgcttcgtgcgcttctgcaacgccttctcgatcccgatcgtcaccttcgtggacgtgccgggcttcctgccgggcacggcgcaggaatatggcggcctgatcaagcacggcgccaagctgctcttcgcctacagccaagccaccgtgccgctcgtgaccatcatcacccgcaaggccttcggcggcgcctacgacgtcatggcctccaagcatgtcggcgccgacctgaactacgcgtggccgacggcgcagatcgcggtgatgggcgccaagggcgctgtcgagatcatcttccgcgccgagatcggcgatgcggacaagatcgccgagcggaccaaagaatacgaggaccgcttcctctcgcccttcgtggcggcggagcgcggctacatcgacgaggtgatcatgccccactccacccgcaagcggatcgcgcgggcgctcgggatgctgcgcaccaaggagatggagcagccctggaagaagcacgacaacatcccgctctga

SEQ ID NO: 26 - DNA, 2004 bp, Methylorubrum extorquens
  source 1..2004: mol_type = genomic DNA
atgttcgataagatcctgattgccaaccggggcgaaatcgcctgccgtatcatcaagacggcccagaaaatgggcatcaagacggtggcggtctattcggacgccgaccgtgatgcggtccacgtcgcgatggccgacgaggcggtgcatatcggcccggcgcccgctgcgcagtcctaccttctgatcgaaaagatcatcgacgcctgcaagcagaccggcgcccaagcggtccatccgggctacggcttcctttccgagcgcgagtccttccccaaggcgctggcggaagcgggcatcgtctttatcggccccaatccgggtgccatcgccgcaatgggcgacaagatcgaatcgaagaaggccgcggccgcggccgaggtctcgacggtgccgggcttcctcggcgtgatcgagagccccgagcacgccgtgacgatcgccgatgagatcggctatccggtgatgatcaaggcgtcggcgggcggcggcggcaagggtatgcgcatcgccgaatcggccgatgaggtcgccgagggcttcgcccgcgccaagtccgaggcctcgtcctccttcggcgacgaccgcgtcttcgtggaaaagttcatcaccgacccgcgccacatcgagatccaggtgatcggcgataagcacggcaacgtgatctatctcggtgagcgcgagtgctcgatccagcgccgcaaccagaaggtcatcgaggaggcgccgtcgccgctcctcgacgaagagacgcgccgcaagatgggcgagcaggcggtcgcgctcgccaaggccgtgaattacgactccgccggcaccgtcgagttcgtcgccggccaggacaagtcgttctacttcctcgaaatgaacacccgcctgcaggtggagcacccggtcaccgagatgatcaccgggctcgacctcgtcgagctgatgatccgggtggccgccggcgagaagctgccgctgtcgcaggatcaggtgaagctcgacggctgggcggtcgagagccgcgtctatgccgaggatccgacccgcaacttcctgccctcgatcggtcggctgactacctaccagccgccggaggagggcccgctcggcggggcgatcgtgcgcaacgataccggcgtggaggagggcggcgagatcgcgatccactacgatccgatgattgccaagctcgtaacctgggcgccgacccggttggaagccatcgaagcgcaggcgaccgcgctcgacgccttcgccatcgagggcatccgccacaacatccccttcctcgccaccctgatggcccatccccgctggcgcgacggccggctctcgacgggcttcatcaaggaagagttccccgaaggcttcatcgcacccgagcccgaggggccggtcgctcatcggctcgcggcggtggcggcggcgatcgatcacaagctcaacatccgcaagcgcggcatctccggccagatgcgcgacccgagcctgctgaccttccagcgcgagcgcgtggtggtgctctccggccagcgcttcaacgtcaccgtcgatcctgacggcgacgacctcctcgtcaccttcgacgacggtacgacagccccggtgcgcagcgcgtggcgccccggtgcgccggtctggagcggtacggtcggagatcagtcggtcgcgatccaggtgcgtccgctcctcaacggtgtgttcctgcagcatgcgggcgcggcggcggaagcgcgggtgttcacccgccgcgaggccgaactcgccgacctgatgccggtcaaggagaatgccggctccggcaagcagctcctttgcccgatgcccggcctggtcaagcagatcatggtcagcgagggccaggaggtgaagaacggcgagccgctggccatcgtcgaggcgatgaagatggagaacgtgctgcgcgccgaacgcgacggcaccatctccaagatcgccgccaaggaaggcgacagcctcgccgtcgatgccgtgatcctggaattcgcctga

SEQ ID NO: 27 - protein, 279 aa, Methylorubrum extorquens
MQFRLSQAARSEGHRLHSHDRLDSTNSEAMRLAQGGETGPLWVTTQRQEAGRGRRGNAWTSPEGNLAASLLMPVAGVAPEMVATLGFVAGVALVDALRDACRLALSPRAEMGDAPLPQGIAPSAAAIHLKWPNDVLADGQKLAGILLEAETLPGGRRAVVVGFGVNVAAAPDGLPYPAAALAAFSAADAPMLLEFLSERFVEAVRIWNKGRGFSNIRRRWLERAAGVGAPVSVRMAEVTLTGIFETIDEGGRLVILAPDGTRRTVTAGEVHFGSAATAA

SEQ ID NO: 28 - DNA, 840 bp, Methylorubrum extorquens
  source 1..840: mol_type = genomic DNA
atgcagttccggctaagtcaggcggcccggtccgaggggcatcggctccacagccacgaccggctcgactcgaccaacagcgaggccatgcgcctcgcccagggcggcgagacgggcccgctctgggtcacgacccaacgccaggaggcgggccgcggccgacgcggcaacgcctggacctcgccggagggcaacctcgccgcgagcctgctgatgccggtggccggcgtggcgccggagatggtggccacgctgggcttcgtggcgggcgtggcgttggtggacgctctgcgcgatgcgtgccgtttagccctctccccgcgtgcggagatgggggatgcaccgctgccgcagggcattgctcctagcgctgccgccatccatctgaaatggcccaacgacgtcctcgccgacggccaaaaactcgccggcatcctgctggaggccgagacgctgccgggcgggcggcgggcggtggtggtcggtttcggcgtcaacgtcgcggcggcgccggacggtttgccctatccggcggcggcgcttgcggctttttccgcggcggatgcgccgatgctgctcgaattcctgtcggaacggtttgtcgaagccgtccggatctggaacaaggggcgcggattctcgaatatccggcggcggtggctggagcgcgcggcgggggtaggggcccccgtgtcggttcgcatggccgaagtcacgctgacgggcatctttgaaacgatcgacgagggggggcggctcgtgatcctcgccccggacggaacccgacgaaccgtgacggcgggcgaggtgcatttcggaagtgccgcgacggcggcctga

SEQ ID NO: 29 - DNA, 21 bp, synthetic construct
  source 1..21: mol_type = other DNA
cgtccacgaaggtgacgatcg

SEQ ID NO: 30 - DNA, 47 bp, synthetic construct
  source 1..47: mol_type = other DNA
ccatatgaaggacatcctcgagaagtttgaggagcgtcgcgcacagg

SEQ ID NO: 31 - DNA, 40 bp, synthetic construct
  source 1..40: mol_type = other DNA
gcacaggcccgtctcggcggccgtgaaaagcggctcgagg

SEQ ID NO: 32 - DNA, 49 bp, synthetic construct
  source 1..49: mol_type = other DNA
cacgggtcgttcgaggagttcgacgaattcgtgcagcaccgctccaccg

SEQ ID NO: 33 - DNA, 49 bp, synthetic construct
  source 1..49: mol_type = other DNA
cacgggtcgttcgaggagttcgaccagttcgtgcagcaccgctccaccg

SEQ ID NO: 34 - DNA, 50 bp, synthetic construct
  source 1..50: mol_type = other DNA
gcagcaccgctccaccgatttcggccgtgagaagcagaagatccccggcg

SEQ ID NO: 35 - DNA, 43 bp, synthetic construct
  source 1..43: mol_type = other DNA
cacggtgttcggcggctcgaactccgaggcgcacgcagccaag

SEQ ID NO: 36 - DNA, 50 bp, synthetic construct
  source 1..50: mol_type = other DNA
caaggccttcggcggcgcctacgtcgtcatggcctccaagcatgtcggcg

SEQ ID NO: 37 - DNA, 44 bp, synthetic construct
  source 1..44: mol_type = other DNA
gggcgctcgggatgctgcgcaacaaggagatggagcagcccagg

SEQ ID NO: 38 - DNA, 50 bp, synthetic construct
  source 1..50: mol_type = other DNA
gcaccaaggagatggagcagccccataagaagcacgacaacatcccgctc

SEQ ID NO: 39 - DNA, 50 bp, synthetic construct
  source 1..50: mol_type = other DNA
cccaggaagaagcacgacaacatcccgttctgagaattcgagctccgtcg

SEQ ID NO: 40 - DNA, 40 bp, synthetic construct
  source 1..40: mol_type = other DNA
gcacaggcccgtctcggcggcgcggaaaagcggctcgagg

SEQ ID NO: 41 - DNA, 40 bp, synthetic construct
  source 1..40: mol_type = other DNA
gcacaggcccgtctcggcggccatgaaaagcggctcgagg

SEQ ID NO: 42 - DNA, 40 bp, synthetic construct
  source 1..40: mol_type = other DNA
gcacaggcccgtctcggcggcaaagaaaagcggctcgagg

SEQ ID NO: 43 - DNA, 40 bp, synthetic construct
  source 1..40: mol_type = other DNA
gcacaggcccgtctcggcggctatgaaaagcggctcgagg

SEQ ID NO: 44 - DNA, 40 bp, synthetic construct
  source 1..40: mol_type = other DNA
gcacaggcccgtctcggcggcndtgaaaagcggctcgagg

SEQ ID NO: 45 - DNA, 40 bp, synthetic construct
  source 1..40: mol_type = other DNA
gcacaggcccgtctcggcggcvhggaaaagcggctcgagg

SEQ ID NO: 46 - DNA, 40 bp, synthetic construct
  source 1..40: mol_type = other DNA
gcacaggcccgtctcggcggctgggaaaagcggctcgagg

SEQ ID NO: 47 - DNA, 43 bp, synthetic construct
  source 1..43: mol_type = other DNA
cacggtgttcggcggctcgndttccgaggcgcacgcagccaag

SEQ ID NO: 48 - DNA, 43 bp, synthetic construct
  source 1..43: mol_type = other DNA
cacggtgttcggcggctcgvhgtccgaggcgcacgcagccaag

SEQ ID NO: 49 - DNA, 43 bp, synthetic construct
  source 1..43: mol_type = other DNA
cacggtgttcggcggctcgttgtccgaggcgcacgcagccaag

SEQ ID NO: 50 - DNA, 21 bp, synthetic construct
  source 1..21: mol_type = other DNA
gagcggataacaattcccctg

SEQ ID NO: 51 - DNA, 19 bp, synthetic construct
  source 1..19: mol_type = other DNA
ccaggacatggcgctgaag

SEQ ID NO: 52 - DNA, 21 bp, synthetic construct
  source 1..21: mol_type = other DNA
gaaggcgacttcttcgagatc

SEQ ID NO: 53 - DNA, 16 bp, synthetic construct
  source 1..16: mol_type = other DNA
gatgctgcgcaccaag

SEQ ID NO: 54 - DNA, 16 bp, synthetic construct
  source 1..16: mol_type = other DNA
caatccgggtgccatc

SEQ ID NO: 55 - DNA, 16 bp, synthetic construct
  source 1..16: mol_type = other DNA
gaacacccgcctgcag

SEQ ID NO: 56 - DNA, 20 bp, synthetic construct
  source 1..20: mol_type = other DNA
cgatcgatcacaagctcaac

SEQ ID NO: 57 - DNA, 42 bp, synthetic construct
  source 1..42: mol_type = other DNA
gtttaactttaataaggagatataccatgggcagcagccatc

SEQ ID NO: 58 - DNA, 40 bp, synthetic construct
  source 1..40: mol_type = other DNA
gattactttctgttcgacttaagcattatgcggccgcaag

SEQ ID NO: 59 - DNA, 19 bp, synthetic construct
  source 1..19: mol_type = other DNA
gcgacgctcctcaagcttc

SEQ ID NO: 60 - DNA, 23 bp, synthetic construct
  source 1..23: mol_type = other DNA
aagtccttcgagaacaggaagac